(19) United States
(12) Patent Application Publication
(10) Pub. No.: US 2004/0170622 A1
(43) Pub. Date: Sep. 2, 2004

(54) Methods and compositions for modulating XBP-1 activity

(75) Inventors: Laurie H. Glimcher, West Newton, MA (US); Ann-Hwee Lee, Chestnut Hill, MA (US); Neil Iwakoshi, Los Alamitos, CA (US)

Correspondence Address: LAHIVE & COCKFIELD, LLP., 28 STATE STREET, BOSTON, MA, 02109, US

(73) Assignee: President and Fellows of Harvard College, Cambridge, MA

(21) Appl. No.: 10/655,620

(22) Filed: Sep. 2, 2003

Related U.S. Application Data
(60) Provisional application No. 60/407,166, filed on Aug. 30, 2002.
(60) Provisional application No. 60/488,568, filed on Jul. 18, 2003.

(51) Int. Cl. A61K 39/395; G01N 33/53; G01N 33/543

(52) U.S. Cl. 424/130.1; 435/7.1; 436/518

(57) ABSTRACT

The invention provides methods and compositions for modulating the expression, processing, post-translational modification, and/or activity of XBP-1 protein, or a protein in a signal transduction pathway involving XBP-1. Exemplary XBP-1 activities that can be modulated using the methods and compositions of the invention include: the Unfolded Protein Response (UPR), plasma cell differentiation, immunoglobulin production, apoptosis and the production of IL-6. The present invention also pertains to methods for identifying compounds that modulate the expression, processing, post-translational modification, and/or activity of XBP-1 protein or a molecule in a signal transduction pathway involving XBP-1.

### RELATED APPLICATIONS

This application claims priority to U.S. Provisional Application Serial No. 60/407,166, titled “Methods and Compositions for Modulating XBP-1 Activity” filed Aug. 30, 2002, and U.S. Provisional Application Serial No. 60/488,568 titled “Methods and Compositions for Modulating XBP-1 Activity” filed Jul. 18, 2003 the entire contents of these applications are incorporated herein by this reference.

### GOVERNMENT FUNDING

Work described herein was supported, at least in part, under grant AI 32412 awarded by the National Institutes of Health. The U.S. government, therefore, may have certain rights in this invention.

### BACKGROUND OF THE INVENTION

XBP-1 is expressed ubiquitously in adults but is mainly found in exocrine glands and bone precursors in the embryonic mouse (Liou et al. 1990, Science 247:1581-1584; Clauss et al. 1993, Dev. Dynamics 197:146-156). Ih vitro studies have demonstrated downregulation of the XBP-1 gene by BSAP, dimerization of XBP-1 protein with c-Fos, and a decrease in MHC class II gene expression when antisense XBP-1 sequences are introduced into Raji cells (Reimold et al. 1996, J. Exp. Med. 183:393-401; Ono et al. 1991, Proc. Natl. Acad. Sci. USA 88:4309-4312). XBP-1 is the first transcription factor shown to be selectively and specifically required for the terminal differentiation of B lymphocytes to plasma cells. XBP-1 transcripts are rapidly upregulated in vitro by stimuli that induce plasma cell differentiation and XBP-1 is found at high levels in normal plasma cells.

However, the signaling pathways in which XBP-1 is involved and the molecular mechanisms by which XBP-1 is induced, remain largely unknown. Further elucidation of the role of XBP-1 in cells would be of considerable benefit in identifying targets for drug discovery and in providing methods for modulating cellular pathways in which XBP-1 is involved.

### SUMMARY OF THE INVENTION

The present invention demonstrates, inter alia, a role for the transcription factor XBP-1 in the activation of the Unfolded Protein Response (UPR). The UPR is a signaling pathway that ensures that cells can handle the proper folding of proteins. The UPR was described over a decade ago in studies that examined the proximal signals responsible for induction of the stress proteins GRP78 and GRP94. Over-expression of misfolded proteins in the ER was found to be a primary signal for the increased production of these molecular chaperones (Kozutsumi et al (1988) Nature). Although the role of the UPR in cells undergoing stress from environmental stimuli or exposed to drugs that disrupt homeostasis has become clearer, the role of the UPR in plasma cell differentiation had not been described.

The present invention is based, at least in part, on the finding that signals that induce cellular differentiation and the unfolded protein response (UPR) cooperate to induce both XBP-1 mRNA expression and splicing of the resulting XBP-1 transcript. In addition, XBP-1 has been found to regulate transcription of a number of genes and to regulate a variety of cellular responses.

Accordingly, in one aspect, the invention pertains to methods of identifying compounds useful in modulating a biological activity of XBP-1 comprising, a) providing an indicator composition comprising mammalian XBP-1 protein; b) contacting the indicator composition with each member of a library of test compounds; c) selecting from the library of test compounds a compound of interest that modulates the expression, processing, post-translational modification, and/or activity of XBP-1 protein; to thereby identify a compound that modulates a biological activity of XBP-1.

In one embodiment, the method further comprises measuring the effect of the compound on the biological activity of XBP-1.

In one embodiment, the biological activity is selected from the group consisting of: modulation of the UPR, modulation of cellular differentiation, modulation of IL-6 production, modulation of immunoglobulin production, modulation of the proteasome pathway, modulation of protein folding and transport, modulation of terminal B cell differentiation, and modulation of apoptosis.

In one embodiment, the post-translational modifications are selected from the group consisting of phophorylation, glycosylation and ubiquitination is modulated.

In one embodiment, the activity of XBP-1 is measured by measuring the binding of XBP-1 to IRE-1 or ATF6α.

In another embodiment, the activity of XBP-1 is measured by measuring the binding of XBP-1 to a regulatory region of a gene responsive to XBP-1.

In one embodiment, the gene is a chaperone gene. In another embodiment, the gene is selected from the group consisting of ERdj4, p58^{ipk}, EDEM, PDI-P5, RAMP4, HEDJ, BiP, ATF6α, XBP-1, Armet and DNAJB9.

In one embodiment, the activity of XBP-1 is measured by measuring the production of a protein. In on e embodiment, the protein is selected from the group consisting of α-fetoprotein, α1-antitrypsin, and albumin. In one embodiment, the protein is an immunoglobulin.

In one embodiment, the activity of XBP-1 is measured by measuring IL-6 expression.

In another embodiment, the indicator composition is a cell that expresses XBP-1 protein.

In another embodiment, the cell has been engineered to express the XBP-1 protein by introducing into the cell an expression vector encoding the XBP-1 protein.

In yet another embodiment, the indicator composition is a cell free composition.

In still another embodiment, the indicator composition is a cell that expresses an XBP-1 protein and a target molecule, and the ability of the test compound to modulate the interaction of the XBP-1 protein with a target molecule is monitored.

In another embodiment, the indicator composition comprises an indicator cell, wherein the indicator cell comprises an XBP-1 protein and a reporter gene responsive to the XBP-1 protein.

In another embodiment, the indicator cell contains: a recombinant expression vector encoding the XBP-1 protein; and a vector comprising an XBP-1-responsive regulatory element operatively linked a reporter gene; and said method comprises: a) contacting the indicator cell with a test compound; b) determining the level of expression of the reporter gene in the indicator cell in the presence of the test compound; and c) comparing the level of expression of the reporter gene in the indicator cell in the presence of the test compound with the level of expression of the reporter gene in the indicator cell in the absence of the test compound to thereby select a compound of interest that modulates the activity of XBP-1 protein.

In another aspect, the invention pertains to methods of identifying compounds useful in modulating a biological activity of XBP-1 comprising, a) providing an indicator composition comprising mammalian IRE-1 protein; b) contacting the indicator composition with each member of a library of test compounds; c) selecting from the library of test compounds a compound of interest that modulates the expression, processing, post-translational modification, and/or activity of the IRE-1 protein; to thereby identify a compound that modulates a biological activity of XBP-1.

In one embodiment, the biological activity is selected from the group consisting of: modulation of the UPR, modulation of cellular differentiation, modulation of IL-6 production, modulation of immunoglobulin production, modulation of the proteasome pathway, modulation of protein folding and transport, modulation of terminal B cell differentiation, and modulation of apoptosis.

In one embodiment, the activity of IRE-1 is a kinase activity.

In another embodiment, the activity of IRE-1 is an endoribonuclease activity.

In still another embodiment, the activity of IRE-1 is measured by measuring the binding of IRE-1 to XBP-1.

In yet another embodiment, the indicator composition is a cell that expresses IRE-1 protein.

In another embodiment, the cell has been engineered to express the IRE-1 protein by introducing into the cell an expression vector encoding the IRE-1 protein.

In another embodiment, the indicator composition is a cell free composition.

In still another embodiment, the indicator composition is a cell that expresses a mammalian IRE-1 protein and a target molecule, and the ability of the test compound to modulate the interaction of the IRE-1 protein with a target molecule is monitored.

In yet another aspect, the invention pertains to a method of identifying a compound that modulates an XBP-1 biological activity comprising: a) contacting cells deficient in XBP-1 or a molecule in a signaling pathway involving XBP-1 with a test compound; and b) determining the effect of the test compound on the XBP-1 biological activity, the test compound being identified as a modulator of the biological activity based on the ability of the test compound to modulate the biological activity in the cells deficient in XBP-1 or a molecule in a signaling pathway involving XBP-1.

In one embodiment, the cells are in a non-human animal deficient in XBP-1 or a molecule in a signal transduction pathway involving XBP-1 and the cells are contacted with the test compound by administering the test compound to the animal.

In still another aspect, the invention pertains to a method of identifying compounds useful in modulating a biological activity of XBP-1 comprising: a) providing an indicator composition comprising mammalian XBP-1 or a molecule in a signal transduction pathway involving XBP-1; b) contacting the indicator composition with each member of a library of test compounds; c) selecting from the library of test compounds a compound of interest that modulates the expression, processing, post-translational modification, and/or activity of XBP-1 or the molecule in a signal transduction pathway involving XBP-1; to thereby identify a compound that modulates a biological activity of XBP-1 pathway.

In another embodiment, the indicator composition is a cell that expresses XBP-1, IRE-1, PERK, and/or ATF6α protein.

In another embodiment, the cell has been engineered to express the XBP-1, IRE-1, PERK, or ATF6α protein by introducing into the cell an expression vector encoding the XBP-1, IRE-1, PERK or ATF6α protein.

In one embodiment, the indicator composition is a cell free composition.

In another embodiment, the indicator composition is a cell that expresses an XBP-1, IRE-1, PERK, or ATF6α protein and a target molecule, and the ability of the test compound to modulate the interaction of the XBP-1, IRE-1, PERK or ATF6α protein with a target molecule is monitored.

In another aspect, the invention pertains to a method of identifying a compound useful in modulating an autoimmune disease comprising: a) providing an indicator composition comprising XBP-1 or a molecule in a signal transduction pathway involving XBP-1; b) contacting the indicator composition with each member of a library of test compounds; c) selecting from the library of test compounds a compound of interest that downmodulates the expression, processing, post-translational modification, and/or activity of XBP-1 or a molecule in a signal transduction pathway involving XBP-1; to thereby identify a compound that modulates an autoimmune disease.

In another embodiment, the activity of XBP-1 is measured by measuring the binding of XBP-1 to IRE-1 or ATF6α.

In one embodiment, the activity of XBP-1 is measured by measuring the binding of XBP-1 to a regulatory region of a gene responsive to XBP-1.

In one embodiment, the gene is a chaperone gene. In another embodiment, the gene is selected from the group consisting of ERdj4, p58^{ipk}, EDEM, PDI-P5, RAMP4, HEDJ, BiP, ATF6α, XBP-1 and DNAJB9.

In one embodiment, the activity of XBP-1 is measured by measuring the production of a protein. In one embodiment, the protein is selected from the group consisting of α-fetoprotein, albumin, α1-antitrypsin or an immunoglobulin.

In one embodiment, the activity of XBP-1 is measured by measuring IL-6 expression.

In another embodiment, the activity of IRE-1 is measured. In one embodiment, the activity of IRE-1 is a kinase activity. In another embodiment, the activity of IRE-1 is an endoribonuclease activity. In another embodiment, the activity of IRE-1 is measured by measuring the binding of IRE-1 to XBP-1.

In one embodiment, the autoimmune disease is selected from the group consisting of: systemic lupus erythematosus; rheumatoid arthritis; goodpasture's syndrome; Grave's disease; Hashimoto's thyroiditis; pemphigus vulgaris; myasthenia gravis; scleroderma; autoimmune hemolytic anemia; autoimmune thrombocytopenic purpura; polymyositis and dermatomyositis; pernicious anemia; Sjögren's syndrome; ankylosing spondylitis; vasculitis, multiple sclerosis, inflammatory bowel disease, ulcerative colitis, Crohn's disease, and type I diabetes mellitus.

In another embodiment, the autoimmune disease involves the production of an antibody.

In one aspect, the invention pertains to a method of identifying a compound useful in treating a malignancy comprising: a) providing an indicator composition comprising XBP-1 or a molecule in a signal transduction pathway involving XBP-1; b) contacting the indicator composition with each member of a library of test compounds; c) selecting from the library of test compounds a compound of interest that modulates the expression, processing, post-translational modification, and/or activity of XBP-1 or a molecule in a signal transduction pathway involving XBP-1; to thereby identify a compound that modulates a malignancy.

In one embodiment, the activity of XBP-1 is measured by measuring the binding of XBP-1 to IRE-1.

In another embodiment, the activity of XBP-1 is measured by measuring the binding of XBP-1 to a regulatory region of a gene responsive to XBP-1. In one embodiment, the gene is a chaperone gene. In another embodiment, the gene is selected from the group consisting of ERdj4, p58^{ipk}, EDEM, PDI-P5, RAMP4, HEDJ, BiP, ATF6α, XBP-1, Armet and DNAJB9.

In one embodiment, the activity of XBP-1 is measured by measuring the production of a protein. In another embodiment, the protein is selected from the group consisting of α-fetoprotein, albumin, α1-antitrypsin or an immunoglobulin.

In another embodiment, the activity of XBP-1 is measured by measuring IL-6 expression.

In yet another embodiment, the molecule in the signal transduction pathway is IRE-1 and the activity of IRE-1 is measured by measuring a kinase activity.

In another embodiment, the molecule in the signal transduction pathway is IRE-1 and the activity of IRE-1 is an endoribonuclease activity. In another embodiment, the molecule in the signal transduction pathway is IRE-1 and the activity of IRE-1 is measured by measuring the binding of IRE-1 to XBP-1.

In one embodiment, the malignancy is selected from the group consisting of: acute lymphoblastic leukemia; acute myeloid leukemia; adrenocortical carcinoma; AIDS-related lymphoma; cancer of the bile duct; bladder cancer; bone cancer, osteosarcomal malignant fibrous histiocytomal brian stem gliomal brain tumor; breast cancer; bronchial adenomas; carcinoid tumors; adrenocortical carcinoma; central nervous system lymphoma; cancer of the sinus, cancer of the gall bladder; gastric cancer; cancer of the salivary glands; cancer of the esophagus; neural cell cancer; intestinal cancer (e.g., of the large or small intestine); cervical cancer; colon cancer; colorectal cancer; cutaneous T-cell lymphoma; B-cell lymphoma; T-cell lymphoma; endometrial cancer; epithelial cancer; endometrial cancer; intraocular melanoma; retinoblastoma; hairy cell leukemia; liver cancer; Hodgkin's disease; Kaposi's sarcoma; acute lymphoblastic leukemia; lung cancer; non-Hodgkin's lymphoma; melanoma; multiple myeloma; neuroblastoma; prostate cancer; retinoblastoma; Ewing's sarcoma; vaginal cancer; Waldenstrom's macroglobulinemia; adenocarcinomas; ovarian cancer, chronic lymphocytic leukemia, pancreatic cancer; and Wilm's tumor.

In one embodiment, the malignancy is in a secretory cell.

In another aspect, the invention pertains to a method for identifying a compound which modulates an interaction between mammalian XBP-1 and IRE-1 comprising: (a) providing a first polypeptide comprising a IRE-1 interacting portion of an XBP-1 molecule and a second polypeptide comprising an XBP-1 interacting portion of an IRE-1 molecule in the presence and the absence of a plurality of test compounds; and (b) determining the degree of interaction between the first and the second polypeptide in the presence and the absence of a test compound to thereby identify a compound which modulates an interaction between mammalian XBP-1 and IRE-1.

In still another aspect, the invention pertains to a method for identifying a compound which modulates an interaction between mammalian XBP-1 and ATF6α comprising: (a) providing a first polypeptide comprising a ATF6α interacting portion of an XBP-1 molecule and a second polypeptide comprising an XBP-1 interacting portion of an ATF6α molecule in the presence and the absence of a plurality of test compounds; and (b) determining the degree of interaction between the first and the second polypeptide in the presence and the absence of a test compound to thereby identify a compound which modulates an interaction between mammalian XBP-1 and ATF6α.

In another embodiment, the interaction between the first and second peptides is determined by binding of XBP-1 to IRE-1 or ATF6α. In yet another embodiment, the interaction between the first and second peptides is determined by measuring XBP-1 activity.

In one embodiment, the interaction between the first and second peptides is determined by measuring the level of spliced XBP-1.

In another embodiment, the interaction between the first and second peptides is determined by measuring the level of unspliced XBP-1.

In one embodiment, the compound is useful to treat autoimmune diseases.

In another embodiment, the compound is useful to treat malignancies.

In still another embodiment, the compound is useful to modulate a biological activity of XBP-1.

In another aspect, the invention pertains to a recombinant cell comprising an exogenous XBP-1 molecule or a portion thereof comprising the nucleotide sequence of XBP-1 spanning the splice junction, and a reporter gene operably linked to a regulatory region responsive to spliced XBP-1 such that upon splicing of the XBP-1 protein, transcription of the reporter gene occurs.

In another aspect, the invention pertains to a method of detecting the ability of a compound to upmodulate splicing of XBP-1 comprising, contacting the cell of claim 68 with a compound and measuring the expression of the reporter gene in the presence and the absence of the compound, wherein an increase in the level of spliced XBP-1 in the presence of the compound indicates that the compound upmodulates splicing of XBP-1.

In still another aspect, the invention pertains to a method for modulating expression and/or activity of XBP-1 in a cell comprising contacting the cell with an agent that modulates expression and/or activity of a protein that activates XBP-1 to thereby regulate the expression and/or activity of XBP-1.

In one embodiment, the protein that activates XBP-1 is IRE-1.

In one embodiment, the agent is not a proteasome inhibitor of the dipeptidyl boronate class.

In another embodiment, the cell is a cell from a patient identified as one in need of modulation of the UPR.

In still another embodiment, the patient is identified by measuring expression, processing, post-translational modification, and/or activity of XBP-1 protein or a protein in a signal transduction pathway involving XBP-1.

In another aspect, the invention pertains to a method for modulating expression, in a cell, of a gene whose transcription is regulated by XBP-1, comprising contacting the cell with an agent that increases expression, processing, post-translational modification, and/or activity of spliced XBP-1 such that expression of the gene is altered.

In one embodiment, the cell is a cell isolated from or present in a patient identified as one in need of modulation of the UPR.

In one embodiment, the patient is identified by measuring expression, processing, post-translational modification, and/or activity of XBP-1 protein or a protein in a signal transduction pathway involving XBP-1.

In yet another aspect, the invention pertains to a method for modulating expression, in a cell, of a gene whose transcription is regulated by XBP-1, comprising contacting the cell with an agent that increases the ratio of spliced XBP-1 to unspliced XBP-1 such that expression of the gene is altered.

In one embodiment, the cell is a cell isolated from or present in a patient identified as one in need of modulation of the UPR.

In one embodiment, the patient is identified by measuring expression, processing, post-translational modification, and/or activity of XBP-1 protein or a protein in a signal transduction pathway involving XBP-1.

In one embodiment, the step of contacting occurs in vivo in a subject that would benefit from modulation of an XBP-1 biological activity.

In still another aspect, the invention pertains to a method for increasing expression, in a cell, of a gene involved in mediating a biological effect of XBP-1 whose transcription is regulated by XBP-1, comprising contacting a cell with an agent that modulates expression, processing, post-translational modification, and/or activity of XBP-1 in the cell such that expression of the gene is increased.

In one embodiment, the cell is isolated from or present in a patient identified as one in need of modulation of the UPR.

In one embodiment, the patient is identified by measuring expression, processing, post-translational modification, and/or activity of XBP-1 protein or a protein in a signal transduction pathway involving XBP-1.

In a further aspect, the invention pertains to a method for decreasing expression, in a cell, of a gene involved in mediating a biological effect of XBP-1 whose transcription is regulated by XBP-1, comprising contacting a cell with an agent that modulates expression, processing, post-translational modification, and/or activity of XBP-1 in the cell such that expression of the gene is decreased.

In one embodiment, the agent is not a proteasome inhibitor of the dipeptidyl boronate class.

In yet another embodiment, the cell is isolated from or present in a patient identified as one in need of modulation of the UPR.

In one embodiment, the patient is identified by measuring expression, processing, post-translational modification, and/or activity of XBP-1 protein or a protein in a signal transduction pathway involving XBP-1.

In still another aspect, the invention pertains to a method for increasing expression, in a cell, of a gene involved in mediating a biological effect of XBP-1 whose transcription is regulated by XBP-1, comprising contacting a cell with an agent that increases the activity of spliced XBP-1 in the cell such that expression of the gene is increased.

In one embodiment, the cell is isolated from or present in a patient identified as one in need of modulation of the UPR.

In one embodiment, the patient is identified by measuring expression, processing, post-translational modification, and/or activity of XBP-1 protein or a protein in a signal transduction pathway involving XBP-1.

In yet another aspect, the invention pertains to a method for increasing expression, in a cell, of a gene involved in mediating a biological effect of XBP-1 whose transcription is regulated by XBP-1, comprising contacting a cell with an agent that decreases the activity of unspliced XBP-1 in the cell such that expression of the gene is increased.

In one embodiment, the activity of unspliced XBP-1 comprises inhibiting the activity of spliced XBP-1.

In another aspect, the invention pertains to a method for decreasing expression, in a cell, of a gene involved in mediating a biological effect of XBP-1 whose transcription is regulated by XBP-1, comprising contacting a cell with an agent that decreases the activity of spliced XBP-1 in the cell such that expression of the gene is decreased.

In one embodiment, the agent is not a proteasome inhibitor of the dipeptidyl boronate class.

In one embodiment, the cell is isolated from or present in a patient identified as one in need of modulation of the UPR.

In one embodiment, the patient is identified by measuring expression, processing, post-translational modification, and/or activity of XBP-1 protein or a protein in a signal transduction pathway involving XBP-1.

In a further embodiment, the activity of spliced XBP-1 is decreased by introducing a dominant negative XBP-1 protein or nucleic acid molecule that mediates RNAi into the cell in an amount sufficient to inhibit activity of spliced XBP-1.

In another aspect, the invention pertains to a method for decreasing expression, in a cell, of a gene involved in mediating a biological effect of XBP-1 whose transcription is regulated by XBP-1, comprising contacting a cell with an agent that increases the activity of unspliced XBP-1 in the cell such that expression of the gene is decreased.

In one embodiment, the activity of unspliced XBP-1 comprises inhibiting the activity of spliced XBP-1.

In one embodiment, the gene is selected from the group consisting of ERdj4, p58^{ipk}, EDEM, PDI-P5, RAMP4, HEDJ, BiP, ATF6α, XBP-1, Armet and DNAJB9.

In one embodiment, the cell is a B cell.

In another aspect, the invention pertains to a method of modulating at least one XBP-1 biological activity comprising contacting a cell with an agent that increases the expression, processing, post-translational modification, and/or activity of spliced XBP-1 in a cell such that the biological activity is modulated.

In one embodiment, the cell is isolated from or present in a patient identified as one in need of modulation of the UPR.

In one embodiment, the patient is identified by measuring expression, processing, post-translational modification, and/or activity of XBP-1 protein or a protein in a signal transduction pathway involving XBP-1.

In still another aspect, the invention pertains to a method of modulating at least one XBP-1 biological activity comprising contacting a cell with an agent that decreases the expression, processing, post-translational modification, and/or activity of spliced XBP-1 in a cell such that the biological activity is modulated.

In one embodiment, the agent is not a proteasome inhibitor of the dipeptidyl boronate class.

In another embodiment, the cell is isolated from or present in a patient identified as one in need of modulation of the UPR.

In yet another embodiment, the patient is identified by measuring expression, processing, post-translational modification, and/or activity of XBP-1 protein or a protein in a signal transduction pathway involving XBP-1.

In yet another aspect, the invention pertains to a method of modulating cellular differentiation comprising contacting a cell with an agent that increases the expression, processing, post-translational modification, and/or activity of unspliced XBP-1 such that the biological response is modulated.

In one embodiment, the cell is isolated from or present in a patient identified as one in need of modulation of the UPR.

In one embodiment, the patient is identified by measuring expression, processing, post-translational modification, and/or activity of XBP-1 protein or a protein in a signal transduction pathway involving XBP-1.

In another aspect, the invention pertains to a method for downmodulating, in mammalian cells, the level of expression of genes which are activated by extracellular influences which induce a signal transduction pathway involving XBP-1, the method comprising contacting a cell with an agent that reduces the expression, processing, post-translational modification, and/or activity of spliced XBP-1 in the cells such that expression of said genes is reduced.

In one embodiment, the agent is not a proteasome inhibitor of the dipeptidyl boronate class.

In another embodiment, the cell is isolated from or present in a patient identified as one in need of modulation of the UPR.

In one embodiment, the patient is identified by measuring expression, processing, post-translational modification, and/or activity of XBP-1 protein or a protein in a signal transduction pathway involving XBP-1.

In yet another aspect, the invention pertains to a method for upmodulating, in mammalian cells, the level of expression of genes which are activated by extracellular influences which induce a signal transduction pathway involving XBP-1, the method comprising contacting a cell with an agent that increases the expression, processing, post-translational modification, and/or activity of spliced XBP-1 in the cells such that expression of said genes is upmodulated.

In one embodiment, the cell is isolated from or present in a patient identified as one in need of modulation of the UPR.

In another embodiment, the patient is identified by measuring expression, processing, post-translational modification, and/or activity of XBP-1 protein or a protein in a signal transduction pathway involving XBP-1.

In one embodiment, the extracellular influence induces ER stress.

In still another aspect, the invention pertains to a method for downmodulating XBP-1-mediated intracellular signaling in a cell comprising contacting the cell with an agent that downmodulates the expression, processing, post-translational modification, and/or activity of spliced XBP-1 in the cell such that XBP-1 mediated intracellular signaling is downmodulated.

In one embodiment, the agent is not a proteasome inhibitor of the dipeptidyl boronate class.

In another embodiment, the cell is isolated from or present in a patient identified as one in need of modulation of the UPR.

In one embodiment, the patient is identified by measuring expression, processing, post-translational modification, and/or activity of XBP-1 protein or a protein in a signal transduction pathway involving XBP-1.

In still another aspect, the invention pertains to a method for upmodulating XBP-1-mediated intracellular signaling comprising contacting the cell with an agent that upmodulates the expression, processing, post-translational modification, and/or activity of spliced XBP-1 in the cell such that XBP-1 mediated intracellular signaling is upmodulated.

In one embodiment, the cell is isolated from or present in a patient identified as one in need of modulation of the UPR.

In another embodiment, the patient is identified by measuring expression, processing, post-translational modification, and/or activity of XBP-1 protein or a protein in a signal transduction pathway involving XBP-1.

In another aspect, the invention pertains to a method of increasing IL-6 expression in a cell comprising contacting a cell with an agent that increases the activity of spliced XBP-1 in the cell such that IL-6 production is increased.

In still another aspect, the invention pertains to a method of increasing IL-6 production in a cell comprising contacting a cell with an agent that decreases the activity of unspliced XBP-1 in the cell such that IL-6 production is increased.

In still another aspect, the invention pertains to a method of decreasing IL-6 production in a cell comprising contacting a cell with an agent that decreases the activity of spliced XBP-1 in the cell such that IL-6 production is decreased.

In still another aspect, the invention pertains to a method of decreasing IL-6 production in a cell comprising contacting a cell with an agent that increases the activity of unspliced XBP-1 in the cell such that IL-6 production is decreased.

In another aspect, the invention pertains to a method of downmodulating apoptosis in a cell comprising contacting a cell with an agent that increases the expression, processing, post-translational modification, and/or activity of spliced XBP-1 in the cell such that apoptosis is decreased.

In yet another aspect, the invention pertains to a method of upmodulating apoptosis in a cell comprising contacting a cell with an agent that decreases the expression, processing, post-translational modification, and/or activity of spliced XBP-1 in the cell in the cell such that apoptosis is upmodulated.

In one embodiment, the agent is not a proteasome inhibitor of the dipeptidyl boronate class.

In one embodiment, the cell is isolated from or present in a patient identified as one in need of modulation of the UPR.

In another embodiment, the patient is identified by measuring expression, processing, post-translational modification, and/or activity of XBP-1 protein or a protein in a signal transduction pathway involving XBP-1.

In another aspect, the invention pertains to a method of increasing protein folding, transport, and/or secretion comprising contacting a cell with an agent that increases the expression, processing, post-translational modification, and/or activity of spliced XBP-1 in the cell such that the production of the protein is increased.

In one embodiment, the protein is a viral protein.

In one embodiment, the increased protein folding or transport is measured by increased chaperone protein production.

In another embodiment, the protein is selected from the group consisting of α-fetoprotein, albumin, α1-antitrypsin and luciferase.

In yet another embodiment, the protein is exogenous to the cell. In another embodiment, wherein the protein is an immunoglobulin.

In another embodiment, wherein the cell is a B cell. In another embodiment, wherein the cell is a hepatocyte.

In another embodiment, wherein the protein is recombinantly expressed in a cell.

In another aspect, the invention pertains to a method of increasing protein folding or transport comprising contacting a cell with an agent that decreases the expression, processing, post-translational modification, and/or activity of unspliced XBP-1 in the cell such that production of the protein is increased.

In still another aspect, the invention pertains to a method of decreasing protein folding or transport in a cell comprising contacting a cell with an agent that increases the expression, processing, post-translational modification, and/or activity of unspliced XBP-1 in the cell such that production of the protein is decreased.

In one embodiment, the agent is not a proteasome inhibitor of the dipeptidyl boronate class.

In another embodiment, the cell is isolated from or present in a patient identified as one in need of modulation of the UPR.

In another embodiment, the patient is identified by measuring expression, processing, post-translational modification, and/or activity of XBP-1 protein or a protein in a signal transduction pathway involving XBP-1.

In another aspect, the invention pertains to a method of modulating terminal B cell differentiation comprising contacting a cell with an agent that modulates IL-4 induced signaling in a B cell such that XBP-1 induced transcription is modulated, to thereby modulate terminal B cell differentiation.

In one embodiment, the cell is isolated from or present in a patient identified as one in need of modulation of the UPR.

In another embodiment, the patient is identified by measuring expression, processing, post-translational modification, and/or activity of XBP-1 protein or a protein in a signal transduction pathway involving XBP-1.

In yet another aspect, the invention pertains to a method of modulating an XBP-1 biological activity in a cell comprising contacting a cell with an agent that induces terminal B cell differentiation.

In one embodiment, the cell is isolated from or present in a patient identified as one in need of modulation of the UPR.

In one embodiment, the patient is identified by measuring expression, processing, post-translational modification, and/or activity of XBP-1 protein or a protein in a signal transduction pathway involving XBP-1.

In one embodiment, the agent is IL-4.

In another embodiment, the agent acts via the signaling protein, STAT6.

In still another embodiment, the agent is one or more agents selected from the group consisting of: LPS, CD40 and IL-4.

In one aspect, the invention pertains to a method of treating or preventing a disorder that could benefit from treatment with an agent that downmodulates the activity of spliced XBP-1 or a molecule in a signal transduction pathway involving XBP-1 in a subject comprising administering to the subject with said disorder an agent that downmodulates the expression, processing, post-translational modification, and/or activity of XBP-1 or a molecule in a signal transduction pathway involving XBP-1.

In one embodiment, the patient is identified by measuring expression, processing, post-translational modification, and/or activity of XBP-1 protein or a protein in a signal transduction pathway involving XBP-1.

In another embodiment, the agent modulates the ratio of unspliced XBP-1 to spliced XBP-1.

In another embodiment, the disorder is an autoimmune disease. In still another embodiment, the disorder is a malignancy.

In yet another aspect, the invention pertains to a method of treating or preventing a malignancy comprising administering to the subject with said malignancy an agent that downmodulates the expression, processing, post-translational modification, and/or activity of XBP-1 or a molecule in a signal transduction pathway involving XBP-1 further comprising administering an additional agent useful in treating the malignancy.

In one embodiment, the additional agent is a proteasome inhibitor of the dipeptidyl boronate class.

In a further aspect, the invention pertains to a method of treating or preventing a disorder that could benefit from treatment with an agent that upmodulates the activity of spliced XBP-1 or a molecule in a signal transduction pathway involving XBP-1 in a subject comprising administering to the subject with said disorder an agent that upmodulates the expression, processing, post-translational modification, and/or activity of XBP-1 or a molecule in a signal transduction pathway involving XBP-1.

In one embodiment, the agent modulates the ratio of unspliced XBP-1 to spliced XBP-1.

In one embodiment, the disorder is an acquired immunodeficiency disorder or an infectious disease.

In still another aspect, the invention pertains to an immunomodulatory composition comprising a nucleic acid molecule encoding spliced XBP-1 and an antigen.

In another aspect, the invention pertains to an immunomodulatory composition comprising a compound that increases spliced XBP-1 activity and an antigen.

In another aspect, the invention pertains to an immunomodulatory composition comprising an inhibitor of spliced XBP-1 and an antigen.

In one embodiment, an the inhibitor is a dominant negative inhibitor of spliced XBP-1 and an antigen.

In another aspect, the invention pertains to a method for modulating an autoimmune disease in a subject comprising administering an immunomodulatory composition.

In still another aspect, the invention pertains to a method for modulating cellular differentiation in a subject comprising administering an immunomodulatory composition.

In another aspect, the invention pertains to a method for enhancing an immune response in a subject comprising administering a nucleic acid molecule encoding spliced XBP-1 to the subject such that the immune response is enhanced.

In yet another aspect, the invention pertains to a method of enhancing an immune response in a subject comprising administering an XBP-1 agonist to the subject such that the immune response is enhanced.

### BRIEF DESCRIPTION OF THE FIGURES

FIG. 1 shows induction of XBP-1 mRNA in naive B cells by IL-4.

FIG. 2 shows transcriptional activation and IRE-1-mediated splicing of XBP-1 mRNA during plasma cell differentiation.

FIG. 3 shows XBP-1 splicing and UPR induction in BCL-1 terminal differentiation.

FIG. 4 shows ectopic expression of the spliced form of XBP-1 enhances IgM secretion in stimulated BCL-1 cells.

FIG. 5 shows exclusive expression of the XBP-1 spliced protein restores Ig Production in XBP-1 deficient B cells.

FIG. 6 shows that the XBP-1 spliced protein induces IL-6 production.

FIG. 7 shows a model of XBP-1, UPR, and plasma cell differentiation.

FIG. 8A shows the cDNA sequence of unspliced murine XBP-1, FIG. 8B shows the protein sequence of unspliced murine XBP-1, FIG. 8C shows the cDNA sequence of spliced murine XBP-1 and FIG. 8D shows the protein sequence of spliced murine XBP-1.

FIG. 9 shows an exemplary screening vector for detection of agents which modulate the splicing of XBP-1 mRNA.

FIG. 10 shows that proteasome inhibitors induce ER stress and caspase-12 activation, but suppress the UPR. (A) BiP and CHOP induction was examined by northern blot analysis after treating NIH3T3 or J558 myeloma cells with either MG-132 (20 μM), Tm (10 μg/ml) or both. Cells were pretreated with MG-132 for 1 hr and then further treated with Tm for 4 hrs. Ethidium bromide staining of the gel is shown at the bottom. (B) Inhibition of caspase-12 processing by proteasome inhibitors. Processing of full-length caspase-12 was examined by Western blotting in J558 myeloma cells treated with thapsigargin (1 μM) or PIs (20 μM) during the indicated time periods. (C) Alteration in the ratio of XBP-1 protein species in J558 cells were treated with increasing amounts of MG-132 for 16 hrs. Cells undergoing apoptosis were counted by Annexin V staining. (D) Time course of the XBP-1s to XBP-1u shift. Cells were treated with MG-132 (1 μM) for the indicated times and XBP-1u and spliced protein levels and cell death determined. (E) Alteration in the ratio of XBP-1 species in the MM.1s human myeloma line. Cells were treated with PS-341 (8 nM) in a time course analysis and XBP-1 protein species quantified.

FIG. 11 shows the effect of proteasome inhibitors on IRE-1α-mediated XBP-1 mRNA splicing. (A) XBP-1 mRNA levels in ER stressed J558 cells treated with Tm for 4 hrs in the absence or presence of MG-132. Cells were pretreated with MG-132 for 1 hr before adding Tm. XBP-1 mRNA levels were determined by Northern blot analysis. (B) The ratio of XBP-1u and XBP-1s mRNA was revealed by RT-PCR analysis with a probe set spanning the spliced-out region as demonstrated previously (Iwakoshi et al. 2003. Nature Immunology 4:321-329). (C) Effect of a panel of PIs on XBP-1 splicing. Cells were treated with Tm for 4 hrs in the absence or presence of MG-132 (10 μM), PS-341 (10 μM), Lactacystin (10 μM), ZL₃VS (50 μM) or AdaAhxL₃VS (50 μM), and XBP-1 mRNA splicing measured by RT-PCR analysis. (D) IRE-1α phosphorylation in NIH3T3 cells were treated with Tm as indicated after 2 hrs of pretreatment with MG-132 (10 μM).

FIG. 12 shows that proteasome inhibitors stabilize XBP-1u protein to act as a dominant negative inhibitor of XBP-1s activity. (A) Ubiquitination of XBP-1 in HeLa cells were cotransfected with XBP-1u and His-tagged ubiquitin expression plasmids. (B) Degradation rates of XBP-1u and XBP-1s proteins were determined by pulse labeling J558 cells with ³⁵S Met/Cys for 1 hr and chasing for the indicated times. (C) Effect of XBP-1u on XBP-1s dependent UPRE activation in PI-treated NIH3T3 cells with 8-fold excess of XBP-1u plasmids. Transfected cells were treated with MG-132 for 16 hrs before harvesting for luciferase assays. Values represent fold induction of activity compared to the reporter alone after normalizing to Renilla. (D) Generation and expression of lysine to arginine XBP-1u mutants. Two or three lysine residues in the C-terminus of XBP-1u were replaced by arginine to generate XBP-1uKK (235, 252) and XBP-1uKKK(146, 235, 252) by site-directed mutagenesis. dn-XBP contains the N-terminal 188 aa of XBP-1u. Western blot analysis was performed with NIH3T3 extracts transfected with the indicated plasmids. E) Inhibition of XBP-1 dependent activation of the UPRE reporter in NIH 3T3 cells by XBP-1u lysine to arginine mutants.

FIG. 13 shows that cells with an impaired UPR are more sensitive to ER stress induced apoptosis. (A) Synergistic effect of Tm and MG-132 on apoptosis. Annexin V positive cells were counted after treating J558 cells for 18 hrs with suboptimal concentrations of Tm and MG-132 as indicated. (B) J558-iXBP cells were generated by retroviral transduction of J558 cells with the U6 promoter based XBP-1 RNAi vector. (C) XBP-1 dependent gene expression in J558 cells that express control GFP, dn-XBP or iXBP-1 treated with Tm. Generation of dn-XBP-1 J558 cells by infection with a retrovirus containing dn-XBP cDNA inserted into the GFP-RV vector (N. N. lwakoshi et al., Nature Immunology 4, 321-329 (2003)). ERdj4, p58^{IPK}, BiP and CHOP gene expression were examined by Northern blot analysis. (D) Increased apoptosis in iXBP-1 and dn-XBP-1 expressing J558 cells. Cells were treated with the indicated amounts of Tm for 48 hrs and dead cells counted after Annexin V staining.

FIG. 14 shows that treatment of J558 cells with Tm led to an increase in the amount of phosphorylated PERK as assessed using an anti-PERK antibody (a shift upwards in mobility of the PERK species) and, more conclusively, using an antibody that recognizes only phospho-PERK. In the presence of MG-132 a decrease in the autophosphorylation of PERK was observed.

FIG. 15 shows the structure of the XBP-1 gene and protein in wildtype and mutant cells. (A) XBP-1 locus in wild type and XBP-1^{-/-} MEF cells. Splicing of the mutant XBP-1 mRNA in XBP-1^{-/-} cells is shown. * represent termination codons. (B) Wild type and XBP-1^{-/-} MEF cells were untreated or treated with 10 μg/ml Tm for 6 hours. XBP-1 mRNA was revealed by Northern blot analysis. (C) Wild type and XBP-1^{-/-} MEF cells were treated with 10 μM MG-132 or 10 μg/ml Tm for 6 hours. XBP-1u and XBP-1s proteins were detected by Western blot analysis with anti-XBP-1 antibody.

FIG. 16 shows the dependence of UPR target gene expression on XBP-1. (A) Wild type and XBP-1^{-/-} MEF cells were treated with 10 μg/ml Tm for the indicated time periods. Total RNAs were isolated and subjected to Northern blot analysis. The same blot was hybridized sequentially with BiP, CHOP, ERdj4, p58^{ipk}, ATF6α and Gla probes. The p58^{ipk} probe is probe A, from the 5′ end of the gene. Ethidium bromide staining of the gel before blotting is shown at the bottom for loading control. (B) All isoforms of p58^{ipk} are XBP-1 dependent. Here, Northern blot analysis was performed with probe B which recognizes sequences at the 3′ end of the gene. (C) XBP-1-dependent genes are also IRE1α-dependent. Northern blot analysis of RNA prepared from IRE 1α^{-/-} MEFs treated with Tm for varying time periods and assessed for expression of ERdj4 and p58^{ipk}. (D) The ERdj4GL3 reporter was transfected with or without XBP-1s plasmid into wild type and XBP-1^{-/-} MEF cells. Cells were treated with Tm at 1 μg/ml for 16 hours before harvesting as indicated. Luciferase activity was normalized to the Renilla activity. (E) Induction of BiP, ERdj4 and p58^{ipk} in primary B cells by LPS. B220+ primary B cells were isolated from spleens of wildtype or XBP-1^{-/-} RAG2^{-/-} lymphoid chimeras. Cells were untreated or stimulated for three days with 20 μg/ml LPS. Expression of BiP, ERdj4 and p58^{ipk} was determined by Northern blot analysis

FIG. 17 shows the induction of UPR target genes by XBP-1s. Panel A shows MEF-tet-off and MEF-tet-off-XBP-1s cells cultured in media containing 1 μg/ml doxicycline. XBP-1s expression was induced by culturing the cells for three days in doxycycline free media or by treating with Tm for the indicated time. XBP-1s protein was revealed by anti-XBP-1 antibody in western blot analysis. Total RNA was also prepared to measure the expression level of BiP, CHOP, ERdj4, p58^{ipk} and ATF6α mRNA. (B) Additional XBP-1 dependent target genes identified in gene profiling experiments (Table 2) confirmed by Northern analysis in both XBP-1s MEF-tet-off and XBP-1^{-/-} MEFs. Ethidium bromide staining of the gels before blotting are shown at the bottom.

FIG. 18 shows that UPR target gene expression is largely unaffected in the absence of ATF6α and β. (A) Western blot analysis of iATF6α, iATF6β and double iATF6α/β MEFs. iATF6α cells were generated by transfecting MEF cells with U6-iATF6α plasmid which expresses siRNA for ATF6α under the control of the U6 promoter. iATF6β cells and iATF6α/β double knockdown cells were generated using a U6-iATF6β or PNA plasmid to transfect wt or iATF6α MEFs as above. Lysates from wt and iATF6α,β and α/β MEFs untreated or treated with 10 μg/ml Tm for 6 hours were analyzed for the expression of XBP-1 and ATF6α and ATF6β. * indicates nonspecific band recognized by anti-ATF6α antibody. (B) 5×ATF6GL3 or ERSE reporters were transfected into wt, XBP-1^{-/-}, iATF6α, iATF6β, double iATF6α/β and double XBP-1−/− iATF6α MEF cells. Cells were treated with Tm at 1 μg/ml for 16 hours before harvesting as indicated. Luciferase activity was normalized to renilla activity. Fold induction of relative luciferase activity by Tm treatment compared to untreated samples is also shown. (C) Total RNA was prepared to measure the expression level of BiP, CHOP, ERdj4 and p58^{ipk} and Grp94 mRNAs. Ethidium bromide staining of the gel before blotting is shown at the bottom.

FIG. 19 shows UPR target gene expression in cells that lack both XBP-1 and ATF6α. (A) An XBP-1/ATF6α double deficient MEF cell line was generated by transferring siRNA for ATF6α into XBP-1^{-/-} MEF cells. ATF6α protein was absent in the double deficient cells as confirmed by Western blot analysis with anti ATF6α antibody. (B) Total RNA were isolated from the indicated cell lines that were untreated or treated with Tm for 6 hours and subjected to Northern blot analysis. The same blot was hybridized sequentially with BiP, CHOP, ERdj4, p58^{ipk} and Grp94 probes. Ethidium bromide staining of the gel before blotting is shown at the bottom as loading control.

FIG. 20 shows physical and functional interaction between XBP-1 and ATF6. (A) 5×ATF6GL3 and 4×XBPGL3 have five tandem ATF6 or four XBP-1 binding sites upstream of a minimal promoter. These ATF6 and XBP-1 reporter plasmids were cotransfected with either pCGNATF6 or XBP-u/s plasmid that expresses XBP-1s upon Tm treatment. Luciferase assays were performed as described in the legend to FIG. 4.(B) 293T cells were cotransfected with the indicated plasmids. Immunoprecipitation was performed using anti-HA antibody and lysates immunoblotted with anti-XBP-1 antibody. (C) XBP-1 and ATF6α synergistically activate a UPRE reporter. The UPRE (5×ATF6GL3) reporter plasmid was cotransfected with XBP-1s and ATF6α (1-373) expression plasmids individually or at the same time. Luciferase assays were performed as above. (D) ATF6α can transactivate the UPRE in the absence of XBP-1. The UPRE (5×ATF6GL3) reporter plasmid was cotransfected with XBP-1s or ATF6α (1-373) expression plasmids into wt or XBP-1^{-/-} MEFs and luciferase assays performed as above

FIG. 21 shows that dominant negative XBP-1 suppresses both XBP-1 and ATF6α activity. (A) The 5×ATF6GL3 reporter plasmid was cotransfected with either pCGNATF6α or XBP-u/s plasmids into MEF cells with or without the dominant negative XBP-1 expression plasmid. 100 ng DNA was used for each transfection except for pCDNA3.1, which was added to give 1 μg of DNA in total. Luciferase assays were performed as described in the legend to FIG. 18.(B) MEF and MEF-dn-XBP cells that stably express dominant negative XBP-1 protein were treated with 10 μg/ml Tm for the indicated time periods. Total RNAs were isolated and subjected to Northern blot analysis. The same blot was hybridized sequentially with BiP, CHOP, ERdj4, p58^{ipk} and ATF6α probes. Ethidium bromide staining of the gel before blotting is shown at the bottom as loading control.

### DETAILED DESCRIPTION

The instant invention is based, at least in part, on the finding that XBP-1 plays a role in the unfolded protein response in mammalian cells. In addition, the instant examples demonstrate that XBP-1 regulates expression of a variety of different genes and activates IL-6 production. These findings provide for the use of agents that modulate the expression and/or activity of XBP-1 (and other molecules in the pathways in which XBP-1 is involved) for use as drug targets and as targets for therapeutic intervention either alone or in combination with additional agents, e.g., proteasome inhibitors. The instant invention further demonstrates that modulation of XBP-1 has a variety of biological effects, including: modulation of the UPR, modulation of cellular differentiation, modulation of IL-6 production, modulation of immunoglobulin production, modulation of the proteasome pathway, modulation of protein folding and transport, modulation of terminal B cell differentiation, and modulation of apoptosis. These findings provide for the use of XBP-1 (and other molecules in the pathways in which XBP-1 is involved (e.g., IRE-1 and PERK)) as drug targets and as targets for therapeutic intervention in diseases such as malignancies, acquired immunodeficiency and autoimmune disorders. The invention yet further provides immunomodulatory compositions, such as vaccines, comprising agents which modulate XBP-1 activity.

The instant invention further identifies the spliced form of XBP-1 as the form which is active in gene transcription and further shows that the activity of spliced XBP-1 is negatively regulated by the unspliced form. The activity of XBP-1 is shown to be modulated by agents such as proteasome inhibitors, which increase the ratio of unspliced XBP-1 to spliced XBP-1.

In the specific examples provided herein, spliced XBP-1 is shown to activate the UPR in B cells allowing for plasma cell differentiation. XBP-1 is shown to be critical for the survival of myeloma cells, both because of its role in the UPR and also because XBP-1 controls the production of IL-6, a factor critical for myeloma cell survival. Accordingly, the instant invention provides methods of identifying agents that modulate XBP-1, or other molecules in pathways involving XBP-1, as well as methods of modulating the biological effects of XBP-1 or signaling via pathways involving XBP-1.

XBP-1 activation is shown herein to control expression of several other genes, for example, ERdj4, p58^{ipk}, EDEM, PDI-P5, RAMP4, HEDJ, BiP, ATF6α, XBP-1, Armet and DNAJB9, which encodes the 222 amino acid protein, mDj7 (GenBank Accession Number NM_{—}013760 [gi:31560494]). These genes are important in a variety of cellular functions. For example, Hsp70 family proteins including BiP/Grp78 which is a representative ER localizing HSP70 member, function in protein folding in mammalian cells. A family of mammalian DnaJ/Hsp40-like proteins has recently been identified that are presumed to carry out the accessory folding functions. Two of them, Erdj4 and p58^{ipk}, were shown to be induced by ER stress, localize to the ER, and modulate HSP70 activity (Chevalier et al. 2000 J Biol Chem 275: 19620-19627; Ohtsuka and Hata 2000 Cell Stress Chaperones 5: 98-112; Yan et al. 2002 Proc Natl Acad Sci USA 99: 15920-15925). ERdj4 has recently been shown to stimulate the ATPase activity of BiP, and to suppress ER stress-induced cell death (Kurisu et al. 2003 Genes Cells 8: 189-202; Shen et al. 2003 J Biol Chem 277: 15947-15956). ERdj4, p58^{IPK}, EDEM, RAMP-4, PDI-P5 and HEDJ, all appear to act in the ER. ERdj4 (Shen et al. 2003), p58^{IPK} (Melville et al. 1999 J Biol Chem 274: 3797-3803) and HEDJ (Yu et al. 2000 Mol Cell 6: 1355-1364) are localized to the ER and display Hsp40-like ATPase augmenting activity for the HspTO family chaperone proteins. EDEM was shown to be critically involved in the ERAD pathway by facilitating the degradation of ERAD substrates (Hosokawa et al. 2001 EMBO Rep 2:415-422; Molinari et al. 2003 Science 299 1397-1400; Oda et al. 2003 Science 299:1394-1397; Yoshida et al. 2003 Dev. Cell. 4:265-271). RAMP4 is a recently identified protein implicated in glycosylation and stabilization of membrane proteins in response to stress (Schroder et al. 1999 EMBO J 18:4804-4815 ; Wang and Dobberstein 1999 Febs Lett 457:316-322; Yamaguchi et al. 1999 J. Cell Biol 147:1195-1204). PDI-P5 has homology to protein disulfide isomerase, which is thought to be involved in disulfide bond formation (Kikuchi et al. 2002 J. Biochem (Tokyo) 132:451-455). Collectively, these results show that the IRE1/XBP-1 pathway is required for efficient protein folding, maturation and degradation in the ER.

Another UPR signaling pathway is activated by the PERK protein kinase. PERK phosphorylates eIF2α, which induces a transient suppression of protein translation accompanied by induction of transcription factor(s) such as ATF4 (Harding et al. 2000 Mol Cell 6: 1099-1108). eIF2α is also phosphorylated under various cellular stress conditions by specific kinases, double strand RNA activated protein kinase PKR, the amino acid control kinase GCN2 and the heme regulated inhibitor HRI (Samuel 1993 J. Biol. Chem 268:7603-76-6; Kaufman 1999 Genes Dev. 13: 1211-1233). Since genes that are induced by the PERK pathway are also induced by other stress signals, such as amino acid deprivation, it is likely that PERK dependent UPR target genes carry out common cellular defense mechanisms, such as cellular homeostasis, apoptosis and cell cycle (Harding et al. 2003 Mol. Cell 11619-633). Collectively, ER stress activates IRE/XBP-1 and PERK/eIF2α pathways to ensure proper maturation and degradation of secretory proteins and to effect common cellular defense mechanisms, respectively.

The reliance of p58^{IPK} gene expression on XBP-1 connects two of the UPR signaling pathways, IRE1/XBP-1 and PERK. P58^{IPK} was originally identified as a 58 kD inhibitor of PKR in influenza virus-infected kidney cells (Lee et al. 1990 Proc Natl Acad Sci USA 87: 6208-6212) and described to downregulate the activity of PKR by binding to its kinase domain (Katze 1995 Trends Microbiol 3: 75-78). It also has a J domain in the C-terminus which has been shown to participate in interactions with Hsp70 family proteins Melville et al. 1999 J Biol Chem 274: 3797-380). Recently Katze and colleagues have demonstrated that p58^{IPK} interacts with ERK which is structurally similar to PKR, inhibits its eIF2α kinase activity and that it is induced during the UPR by virtue of an ER stress-response element in its promoter region (Yan et al. 2002 Proc Natl Acad Sci USA 99: 15920-15925). The data presented herein indicate that XBP-1 is the transcription factor that controls p58^{IPK} expression during the UPR. This has functional consequences as upregulation of p58^{IPK} upon ER stress may relieve eIF2α phosphorylation and the subsequent change in protein translation induced by PERK in a negative feedback manner.

These genes, and others expressed in response to XBP-1 activation can be therapeutic targets in diseases or disorders in which functional XBP-1 (or a molecule in a signal transduction pathway involving XBP-1) is abnormally expressed, processed, and/or post-translationally modified, and/or when activity of XBP-1 or a molecule in a signal transduction pathway involving is abnormal. Exemplary disorders or diseases include: malignancies, acquired immunodeficiencies, nervous system disorders (e.g., neurodegenerative disorders, mental illness (e.g., bipolar disorder)), type II diabetes, autoimmune disorders, disorders involving reduced protein secretion by cells or accumulation of proteins in the endoplasmic reticulum of cells, disorders that would benefit from modulation of cellular differentiation, disorders that would benefit from modulation of the UPR, disorders that would benefit from modulation of IL-6 production, disorders that would benefit from modulation of immunoglobulin production, disorders that would benefit from modulation of the proteasome pathway, disorders that would benefit from modulation of protein folding and transport, disorders that would benefit from modulation of terminal B cell differentiation, or disorders that would benefit from modulation of apoptosis.

Certain terms are first defined so that the invention may be more readily understood.

I. Definitions

As used herein, the term “XBP-1” refers to a X-box binding human protein that is a DNA binding protein and has an amino acid sequence as described in, for example, Liou, H-C. et. al. (1990) Science 247:1581-1584 and Yoshimura, T. et al. (1990) EMBO J. 9:2537-2542, and other mammalian homologs thereof, such as described in Kishimoto T. et al., (1996) Biochem. Biophys. Res. Commun. 223:746-751 (rat homologue). Exemplary proteins intended to be encompassed by the term “XBP-1” include those having amino acid sequences disclosed in GenBank with accession numbers A36299 [gi:105867], NP_{—}005071 [gi:4827058], P17861 [gi:139787], CAA39149 [gi:287645], and BAA82600 [gi:5596360] or e.g., encoded by nucleic acid molecules such as those disclosed in GenBank with accession numbers AF027963 [gi: 13752783]; NM_{—}013842 [gi:13775155]; or M31627 [gi:184485]. XBP-1 is also referred to in the art as TREB5 or HTF (Yoshimura et al. 1990. EMBO Journal. 9:2537; Matsuzaki et al. 1995. J. Biochem. 117:303).

XBP-1 is a basic region leucine zipper (b-zip) transcription factor isolated independently by its ability to bind to a cyclic AMP response element (CRE)-like sequence in the mouse class II MHC Aα gene or the CRE-like site in the HTLV-1 21 base pair enhancer, and subsequently shown to regulate transcription of both the DRα and HTLV-1 ltr gene.

Like other members of the b-zip family, XBP-1 has a basic region that mediates DNA-binding and an adjacent leucine zipper structure that mediates protein dimerization. Deletional and mutational analysis identified transactivation domains in the C-terminus of XBP-1 in regions rich in acidic residues, glutamine, serine/threonine and proline/glutamine. XBP-1 is present at high levels in plasma cells in joint synovium in patients with rheumatoid arthritis. In human multiple myeloma cells, XBP-1 is selectively induced by IL-6 treatment and implicated in the proliferation of malignant plasma cells.

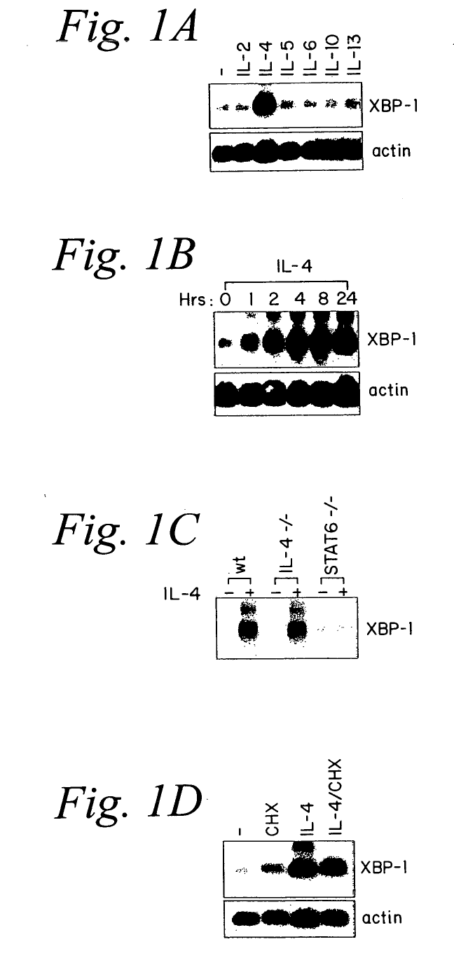

As described above, there are two forms of XBP-1 protein, unspliced and spliced, which differ markedly in their sequence and activity. Unless the form is referred to explicitly herein, the term “XBP-1” as used herein includes both the spliced and unspliced forms. Spliced XBP-1 protein directly controls the activation of the UPR, control plasma differentiation (FIG. 1B) and control the production of the myeloma cell survival cytokine IL-6 (FIG. 1C), while unspliced XBP-1 functions in these pathways only due to its ability to negatively regulate spliced XBP-1.

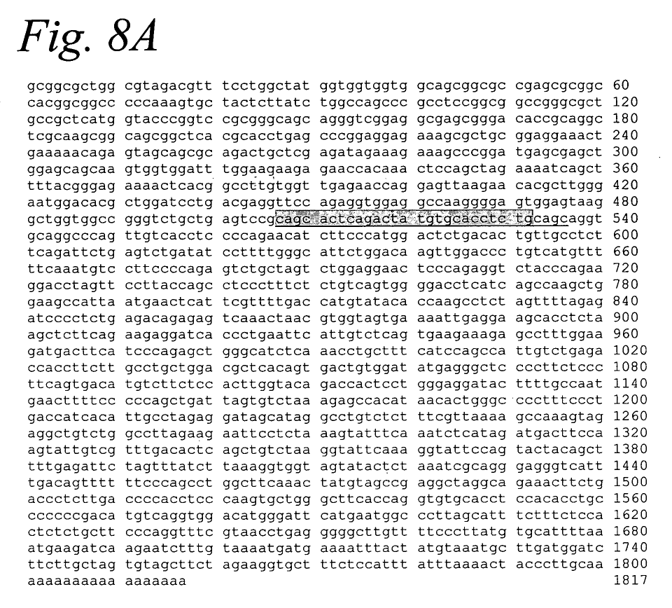

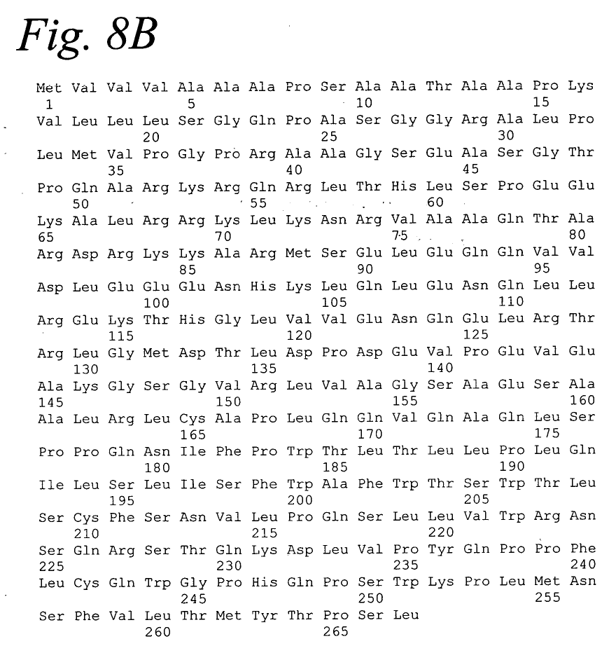

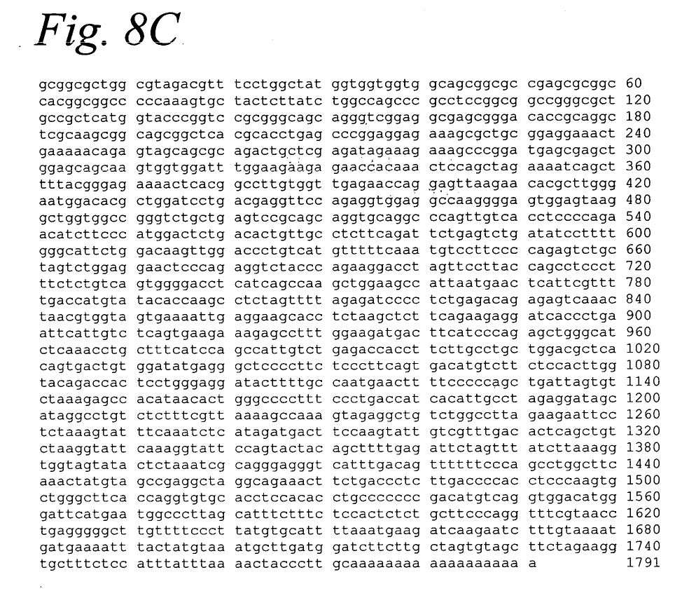

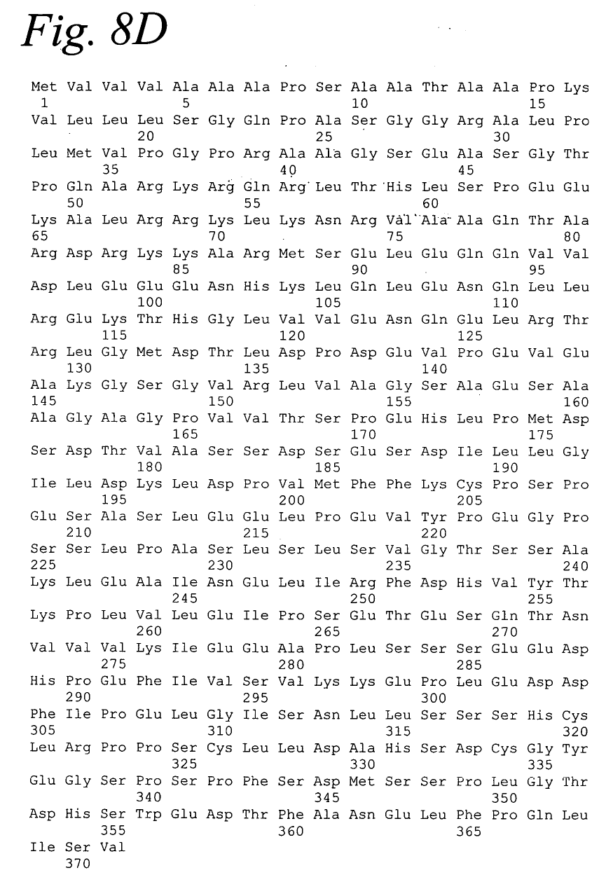

As used herein, the term “spliced XBP-1” refers to the spliced, processed form of the mammalian XBP-1 mRNA or the corresponding protein. Human and murine XBP-1 mRNA contain an open reading frame (ORF1) encoding bZIP proteins of 261 and 267 amino acids, respectively. Both mRNA's also contain another ORF, ORF2, partially overlapping but not in frame with ORF1. ORF2 encodes 222 amino acids in both human and murine cells. Human and murine ORF1 and ORF2 in the XBP-1 mRNA share 75% and 89% identity respectively. In response to ER stress, XBP-1 mRNA is processed by the ER transmembrane endoribonuclease and kinase IRE-1 which excises an intron from XBP-1 mRNA. In murine and human cells, a 26 nucleotide intron is excised. The boundaries of the excised introns are encompassed in an RNA structure that includes two loops of seven residues held in place by short stems. The RNA sequences 5′ to 3′ to the boundaries of the excised introns form extensive base-pair interactions. Splicing out of 26 nucleotides in murine and human cells results in a frame shift at amino acid 165 (the numbering of XBP-1 amino acids herein is based on GenBank accession number NM_{—}013842 [gi:13775155] and one of skill in the art can determine corresponding amino acid numbers for XBP-1 from other organisms, e.g., by performing a simple alignment). This causes removal of the C-terminal 97 amino acids from the first open reading frame (ORF1) and addition of the 212 amino from ORF2 to the N-terminal 164 amino acids of ORF1 containing the b-ZIP domain. In mammalian cells, this splicing event results in the conversion of a 267 amino acid unspliced XBP-1 protein to a 371 amino acid spliced XBP-1 protein. The spliced XBP-1 then translocates into the nucleus where it binds to its target sequences to induce their transcription. The nucleic acid and amino acid sequence of the spliced form of murine XBP-1 are shown in FIGS. 8C and 8D, respectively.

As used herein, the term “unspliced XBP-1” refers to the unprocessed XBP-1 mRNA or the corresponding protein. As set forth above, unspliced murineXBP-1 is 267 amino acids in length and spliced murine XBP-1 is 371 amino acids in length. The sequence of unspliced XBP-1 is known in the art and can be found, e.g., Liou, H-C. et. al. (1990) Science 247:1581-1584 and Yoshimura, T. et al. (1990) EMBO J. 9:2537-2542, or at GenBank accession numbers NM_{—}005080 [gi:14110394] or NM_{—}013842 [gi:13775155]. The nucleic acid and amino acid sequence of the unspliced form of murine XBP-1 are also shown in FIG. 8A.

As used herein, the term “ratio of spliced to unspliced XBP-1” refers to the amount of spliced XBP-1 present in a cell or a cell-free system, relative to the amount or of unspliced XBP-1 present in the cell or cell-free system. “The ratio of unspliced to spliced XBP-1” refers to the amount of unspliced XBP-1 compared to the amount of unspliced XBP-1. “Increasing the ratio of spliced XBP-1 to unspliced XBP-1” encompasses increasing the amount of spliced XBP-1 or decreasing the amount of unspliced XBP-1 by, for example, promoting the degradation of unspliced XBP-1. Increasing the ratio of unspliced XBP-1 to spliced XBP-1 can be accomplished, e.g., by decreasing the amount of spliced XBP-1 or by increasing the amount of unspliced XBP-1. Levels of spliced and unspliced XBP-1 an be determined as described herein, e.g., by comparing amounts of each of the proteins which can be distinguished on the basis of their molecular weights or on the basis of their ability to be recognized by an antibody. In another embodiment described in more detail below, PCR can be performed employing primers with span the splice junction to identify unspliced XBP-1 and spliced XBP-1 and the ratio of these levels can be readily calculated.

As used herein, the term “Unfolded Protein Response” (UPR) or the “Unfolded Protein Response pathway” refers to an adaptive response to the accumulation of unfolded proteins in the ER and includes the transcriptional activation of genes encoding chaperones and folding catalysts and protein degrading complexes as well as translational attenuation to limit further accumulation of unfolded proteins. Both surface and secreted proteins are synthesized in the endoplasmic reticulum (ER) where they need to fold and assemble prior to being transported.

Since the ER and the nucleus are located in separate compartments of the cell, the unfolded protein signal must be sensed in the lumen of the ER and transferred across the ER membrane and be received by the transcription machinery in the nucleus. The unfolded protein response (UPR) performs this function for the cell. Activation of the UPR can be caused by treatment of cells with reducing agents like DTT, by inhibitors of core glycosylation like tunicamycin or by Ca-ionophores that deplete the ER calcium stores. First discovered in yeast, the UPR has now been described in C. elegans as well as in mammalian cells. In mammals, the UPR signal cascade is mediated by three types of ER transmembrane proteins: the protein-kinase and site—specific endoribonuclease IRE-1; the eukaryotic translation initiation factor 2 kinase, PERK/PEK; and the transcriptional activator ATF6. If the UPR cannot adapt to the presence of unfolded proteins in the ER, an apoptotic response is initiated leading to the activation of JNK protein kinase and caspases 7, 12, and 3. The most proximal signal from the lumen of the ER is received by a transmembrane endoribonuclease and kinase called IRE-1. Following ER stress, IRE-1 is essential for survival because it initiates splicing of the XBP-1 mRNA the spliced version of which, as shown herein, activates the UPR.

Eukaryotic cells respond to the presence of unfolded proteins by upregulating the transcription of genes encoding ER resident protein chaperones such as the glucose-regulated BiP/Grp74, GrP94 and CHOP genes, folding catalysts and protein degrading complexes that assist in protein folding. As used herein, the term “modulation of the UPR” includes both upregulation and downregulation of the UPR. As used herein the term “UPRE” refers to UPR elements upstream of certain genes which are involved in the activation these genes in response, e.g., to signals sent upon the accumulation of unfolded proteins in the lumen of the endoplasmic reticulum.

As used herein, the term “ER stress” includes conditions such as the presence of reducing agents, depletion of ER lumenal Ca²⁺, inhibition of glycosylation or interference with the secretory pathway (by preventing transfer to the Golgi system), which lead to an accumulation of misfolded protein intermediates and increase the demand on the chaperoning capacity, and induce ER-specific stress response pathways. ER stress pathways involved with protein processing include the Unfolded Protein Response (UPR) and the Endoplasmic Reticulum Overload Response (EOR) which is triggered by certain of the same conditions known to activate UPR (e.g. glucose deprivation, glycosylation inhibition), as well as by heavy overexpression of proteins within the ER. The distinguishing feature of EOR is its association with the activation of the transcription factor NF-κB. Modulation of both the UPR and the EOR can be accomplished using the methods and compositions of the invention. ER stress can be induced, for example, by inhibiting the ER Ca²⁺ ATPase, e.g., with thapsigargin. As used herein, the term “protein folding or transport” encompasses posttranslational processes including folding, glycosylation, subunit assembly and transfer to the Golgi compartment of nascent polypeptide chains entering the secretory pathway, as well as extracytosolic portions of proteins destined for the external or internal cell membranes, that take place in the ER lumen. Proteins in the ER are destined to be secreted or expressed on the surface of a cell. Accordingly, expression of a protein on the cell surface or secretion of a protein can be used as indicators of protein folding or transport.

As referred to herein, the term “proteasome pathway” refers to a pathway by which a variety of cellular proteins are degraded and is also called the ubiquitin-proteasome pathway. Many proteins are marked for degradation in this pathway by covalent attachment of ubiquitin. For example, as shown in the Examples herein, the XBP-1 unspliced protein is an example of a ubiquitinated, and hence extremely unstable, protein. XBP-1 spliced protein is not ubiquitinated, and has a much longer half life than unspliced XBP-1 protein.

As used herein, the term “IRE-1” refers to an ER transmembrane endoribonuclease and kinase called “iron responsive element binding protein-1,” which oligomerizes and is activated by autophosphorylation upon sensing the presence of unfolded proteins, see, e.g., Shamu et al., (1996) EMBO J. 15: 3028-3039. In Saccharomyces cerevisiae, the UPR is controlled by IREp. In the mammalian genome, there are two homologs of IRE-1, IRE1α and IRE1β. IRE1α is expressed in all cells and tissue whereas IRE1β is primarily expressed in intestinal tissue. The endoribonucleases of either IRE1α and IRE1β are sufficient to activate the UPR. Accordingly, as used herein, the term “IRE-1” includes, e.g., IRE1α, IRE1β and IREp. In a preferred embodiment, IRE-1 refers to IRE1α.

IRE-1 is a large protein having a transmembrane segment anchoring the protein to the ER membrane. A segment of the IRE-1 protein has homology to protein kinases and the C-terminal has some homology to RNAses. Over-expression of the IRE-1 gene leads to constitutive activation of the UPR. Phosphorylation of the IRE-1 protein occurs at specific serine or threonine residues in the protein.

IRE-1 senses the overabundance of unfolded proteins in the lumen of the ER. The oligomerization of this kinase leads to the activation of a C-terminal endoribonuclease by trans-autophosphorylation of its cytoplasmic domains. IRE-1 uses its endoribonuclease activity to excise an intron from XBP-1 mRNA. Cleavage and removal of a small intron is followed by re-ligation of the 5′ and 3′ fragments to produce a processed mRNA that is translated more efficiently and encodes a more stable protein (Calfon et al. (2002) Nature 415(3): 92-95). The nucleotide specificity of the cleavage reaction for splicing XBP-1 is well documented and closely resembles that for IRE-p mediated cleavage of HAC1 mRNA (Yoshida et al. (2001) Cell 107:881-891). In particular, IRE-1 mediated cleavage of murine XBP-1 cDNA occurs at nucleotides 506 and 532 and results in the excision of a 26 base pair fragment (e.g., CAGCACTCAGACTACGTGCACCTCTG (SEQ ID NO:1) for mouse XBP-1; FIG. 8A). IRE-1 mediated cleavage of XBP-1 derived from other species, including humans, occurs at nucleotides corresponding to nucleotides 506 and 532 of murine XBP-1 cDNA, for example, between nucleotides 502 and 503 and 528 and 529 of human XBP-1.

As used herein the term “activating transcription factors 6” include ATF6α and ATF6β. ATF6 is a member of the basic-leucine zipper family of transcription factors. It contains a transmembrane domain and is located in membranes of the endoplasmic reticulum. ATF6 is constitutively expressed in an inactive form in the membrane of the ER. Activation in response to ER stress results in proteolytic cleavage of its N-terminal cytoplasmic domain by the S2P serine protease to produce a potent transcriptional activator of chaperone genes (Yoshida et al. 1998 J. Biol. Chem. 273: 33741-33749; Li et al. 2000 Biochem J 350 Pt 1: 131-138; Ye et al. 2000 Mol Cell 6: 1355-1364; Yoshida et al. 2001 Cell 107: 881-891; Shen et al. 2002 Dev Cell 3: 99-111). The recently described ATF6β is closely related structurally to ATF6α and posited to be involved in the UPR (Haze et al. 2001 Biochem J 355: 19-28; Yoshida et al. 2001b Mol Cell Biol 21: 1239-1248). The third pathway acts at the level of posttranscriptional control of protein synthesis. An ER transmembrane component, PEK/PERK, related to PKR (interferon-induced double-stranded RNA-activated protein kinase) is a serine/threonine protein kinase that acts in the cytoplasm to phosphorylate eukaryotic initiation factor-2α (eIF2α). Phosphorylation of eIF2α results in translation attenuation in response to ER stress (Shi et al. 1998 Mol. Cell. Biol. 18: 7499-7509; Harding et al. 1999 Nature 397: 271-274).

As used herein, “IL-6” refers to a multi-functional cytokine playing a central role in host defense mechanisms. IL-6 functions through interaction with at least two specific receptors on the surface of target cells. The cDNAs for these two receptor chains have been cloned, and they code for two transmembrane glycoproteins: the 80 kDa IL-6 receptor (“IL-6R”) and a 130 kDa glycoprotein called “gp130”. IL-6 interacts with these glycoproteins by a unique mechanism. First, IL-6R binds to IL-6 with low affinity (Kd=about 1 nM) without triggering a signal. The IL-6/IL-6R complex subsequently associates with gp130, which transduces the signal. Gp130 itself has no affinity for IL-6 in solution, but stabilizes the IL-6/IL-6 R complex on the membrane, resulting in high affinity binding of IL-6 (Kd=about 10 pM). Mature human IL-6 is a 185 amino acid polypeptide containing two disulfide bonds and is commercially available. As used herein, the term “modulating IL-6 production” includes either increasing or decreasing IL-6 production in a cell, e.g., in multiple myeloma cells. IL-6 plays a role in a variety of human inflammatory diseases, autoimmune diseases, neoplastic diseases (e.g., multiple myeloma), sepsis, bone resorption (osteoporosis), cachexia, psoriasis, mesangial proliferative glomerulonephritis, renal cell carcinoma, Kaposi's sarcoma, rheumatoid arthritis, hyper gammaglobulinemia, Castleman's disease, IgM gammapathy, cardiac myxoma and autoimmune insulin-dependent diabetes. Accordingly, the present invention is useful in treating disease states associated with IL-6 production.

As used herein, the term “autoimmune disease” refers to disorders or conditions in a subject wherein the immune system attacks the body's own cells, causing tissue destruction. Autoimmune diseases include general autoimmune diseases, i.e., in which the autoimmune reaction takes place simultaneously in a number of tissues, or organ specific autoimmune diseases, i.e., in which the autoimmune reaction targets a single organ. Examples of autoimmune diseases that can be diagnosed, prevented or treated by the methods and compositions of the present invention include, but are not limited to, Crohn's disease; Inflammatory bowel disease (IBD); systemic lupus erythematosus; ulcerative colitis; rheumatoid arthritis; goodpasture's syndrome; Grave's disease; Hashimoto's thyroiditis; pemphigus vulgaris; myasthenia gravis; scleroderma; autoimmune hemolytic anemia; autoimmune thrombocytopenic purpura; polymyositis and dermatomyositis; pernicious anemia; Sjogren's syndrome; ankylosing spondylitis; vasculitis; type I diabetes mellitus; neurological disorders, multiple sclerosis, and secondary diseases caused as a result of autoimmune diseases.

As used herein, the term “malignancy” refers to a non-benign tumor or a cancer. In one embodiment a malignancy expands to other parts of the body as well (metastasizes). A malignant tumor is usually life-threatening, causing death if it remains untreated. If treated, the spread of a malignant tumor can be slowed or even arrested. Depending on the amount of tissue damage prior to treatment, tissue or organ function can be compromised. Examples of malignancies that can be diagnosed, prevented or treated by the methods and compositions of the present invention include, but are not limited to, acute lymphoblastic leukemia; acute myeloid leukemia; adrenocortical carcinoma; AIDS-related lymphoma; cancer of the bile duct; bladder cancer; bone cancer, osteosarcomal malignant fibrous histiocytomal brain stem gliomal brain tumor; breast cancer; bronchial adenomas; carcinoid tumors; adrenocortical carcinoma; central nervous system lymphoma; cancer of the sinus, cancer of the gall bladder; gastric cancer; cancer of the salivary glands; cancer of the esophagus; neural cell cancer; intestinal cancer (e.g., of the large or small intestine); cervical cancer; colon cancer; colorectal cancer; cutaneous T-cell lymphoma; B-cell lymphoma; T-cell lymphoma; endometrial cancer; epithelial cancer; endometrial cancer; intraocular melanoma; retinoblastoma; hairy cell leukemia; liver cancer; Hodgkin's disease; Kaposi's sarcoma; acute lymphoblastic leukemia; lung cancer; non-Hodgkin's lymphoma; melanoma; multiple myeloma; neuroblastoma; prostate cancer; retinoblastoma; Ewing's sarcoma; vaginal cancer; Waldenstrom's macroglobulinemia; adenocarcinomas; ovarian cancer, chronic lymphocytic leukemia, pancreatic cancer; and Wilm's tumor.

In one embodiment, the instant invention is useful in the diagnosis and/or treatment of malignancies originating in the secretory cells of the body. As used herein the term “secretory cell” includes cells specialized for secretion. These cells are usually epithelial in origin and have characteristic, well developed rough endoplasmic reticulum or, in the case of cells secreting lipids or lipid-derived products have well developed smooth endoplasmic reticulum. Exemplary secretory cells include: salivary gland cells, mammary gland cells, lacrimal gland cells, creuminous gland cells, eccrine sweat gland cells, apocrine sweat gland cells, sebaceous gland cells, Bowman's gland cells, Brunner's gland cells, seminal vesicle cells, prostate gland cells, bulbourethral gland cells, Bartholin's gland cells, gland of Littre cells, endometrial cells, goblet cells of the respiratory and digestive tracts, mucous cells of the stomach, zymogenic cells of gastric glands, oxyntic cells of gastric glands, acinar cells of the pancreas, paneth cells of the small intestine, type II pneumocytes of the lung, Clara cells of the lung, anterior pituitary cells, cells of the intermediate pituitary, cells of the posterior pituitary, cells of the gut and respiratory tract, cells of the thyroid gland, cells of the parathyroid gland, cells of the adrenal gland, cells of the testes, cells of the ovaries, cells of the juxtaglomerular apparatus of the kidney, cells secreting extracellular matrix (e.g., epithelial cells, nonepithelial cells (such as fibroblasts, chondrocytes, osteoblasts/osteocytes, osteoprogenitor cells), and secretory cells of the immune system (e.g., Ig producing B cells, cytokine producing T cells, etc).

As used herein, the term “multiple myeloma” refers to a malignancy of the bone marrow in which cancerous plasma cells grow out of control and create a tumor. When these tumors grow in multiple sites, they are referred to as multiple myeloma. Normally, plasma cells make up less than five percent of the cells in bone marrow, but people with multiple myeloma have anywhere from ten percent to more than ninety percent. The overgrowth of malignant plasma cells in bone marrow can cause a number of serious problems throughout the body. Over time, the abnormal cells can permeate the interior of the bone and erode the bone cortex (outer layer). These weakened bones are more susceptible to bone fractures, especially in the spine, skull, ribs, and pelvis.

As used herein, “IL-4” refers to a multi-functional cytokine that is a cofactor in the proliferation of resting B cells stimulated through the cross-linkage of their membrane IgM by anti-IgM antibodies. It is also a T cell factor that induced B-cell differentiation into plasma cells secreting IgG. Hence its early names were B-cell stimulation factor-I (BSF-I), B-cell differentiation factor-I (BCDF-I) and B-cell growth factor-I (BCGF-I). IL-4 exerts different effects on B cells at different stages in the cell cycle. On resting B-cells, IL-4 acts as an activating factor, inducing them to enlarge in size and increase class II MHC expression.

Following activation by an antigen or mitogen, IL-4 acts as a growth factor, driving DNA replication in the B-cells. In the case of proliferating B cells, IL-4 acts as a differentiation factor by regulating class switch to Cepsilon and Cgamma1, i.e., the production of the IgE and IgG1 subclasses. In this role it has been termed a “switch-inducing” factor. IL-4 also plays a major role in T-cell development. It is thought to be influential in promoting differentiation of T helper cells into TH2 cells during an immune response. IL-4 can also act as a mast cell growth factor.

As referred to herein, the term “STAT6” refers to signaling protein linked to the IL-4 receptor. STAT6 is associated with the cytoplamsic domain of CD124 which plays an important role in induction of Th2 T cells and IgE class switch. IL-4 is the ligand for CD124.

As used herein, the various forms of the term “modulate” include stimulation (e.g., increasing or upregulating a particular response or activity) and inhibition (e.g., decreasing or downregulating a particular response or activity).

As used herein, the term “a modulator of XBP-1” includes a modulator of XBP-1 expression, processing, post-translational modification, and/or activity. The term includes agents, for example a compound or compounds which modulates transcription of an XBP-1 gene, processing of an XBP-1 mRNA (e.g., splicing), translation of XBP-1 mRNA, post-translational modification of an XBP-1 protein (e.g., glycosylation, ubiquitination) or activity of an XBP-1 protein. A “modulator of XBP-1 activity” includes compounds that directly or indirectly modulate XBP-1 activity. For example, an indirect modulator of XBP-1 activity can modulate a non-XBP-1 molecule which is in a signal transduction pathway that includes XBP-1. Examples of modulators that directly modulate XBP-1 expression, processing, post-translational modification, and/or activity include antisense or siRNA nucleic acid molecules that bind to XBP-1 mRNA or genomic DNA, intracellular antibodies that bind to XBP-1 intracellularly and modulate (i.e., inhibit) XBP-1 activity, XBP-1 peptides that inhibit the interaction of XBP-1 with a target molecule (e.g., IRE-1) and expression vectors encoding XBP-1 that allow for increased expression of XBP-1 activity in a cell, dominant negative forms of XBP-1, as well as chemical compounds that act to specifically modulate the activity of XBP-1.

As used interchangeably herein, the terms “XBP-1 activity,” “biological activity of XBP-1” or “functional activity XBP-1,” include activities exerted by XBP-1 protein on an XBP-1 responsive cell or tissue, e.g., a hepatocyte, a B cell, or on an XBP-1 nucleic acid molecule or protein target molecule, as determined in vivo, or in vitro, according to standard techniques. XBP-1 activity can be a direct activity, such as an association with an XBP-1-target molecule e.g., binding of spliced XBP-1 to a regulatory region of a gene responsive to XBP-1 (for example, a gene such as ERdj4, p58^{ipk}, EDEM, PDI-P5, RAMP4, HEDJ, BiP, ATF6α, XBP-1; Armet and/or DNAJB9) or the inhibition of spliced XBP-1 by unspliced XBP-1. Alternatively, an XBP-1 activity is an indirect activity, such as a downstream biological event mediated by interaction of the XBP-1 protein with an XBP-1 target molecule, e.g., IRE-1. The biological activities of XBP-1 are described herein and include: e.g., modulation of the UPR, modulation of cellular differentiation, modulation of IL-6 production, modulation of immunoglobulin production, modulation of the proteasome pathway, modulation of protein folding and transport, modulation of terminal B cell differentiation, modulation of apoptosis. These findings provide for the use of XBP-1 (and other molecules in the pathways in which XBP-1 is involved) for as drug targets and as targets for modulation of these biological activities in cells and for therapeutic intervention in diseases such as malignancies, acquired immunodeficiencies and autoimmune disorders. The invention yet further provides immunomodulatory compositions, such as vaccines, comprising agents which modulate XBP-1 activity.

“Activity of unspliced XBP-1” includes the ability to modulate the activity of spliced XBP-1. In one embodiment, unspliced XBP-1 competes for binding to target DNA sequences with spliced XBP-1. In another embodiment, unspliced XBP-1 disrupts the formation of homodimers or heterodimers (e.g., with cfos or ATF6α) by XBP-1.

As used interchangeably herein, “IRE-1 activity,” “biological activity of IRE-1” or “functional activity IRE-1,” includes an activity exerted by IRE-1 on an IRE-1 responsive target or substrate, as determined in vivo, or in vitro, according to standard techniques (Tirasophon et al. 2000. Genes and Development Genes Dev. 2000 14: 2725-2736), IRE-1 activity can be a direct activity, such as a phosphorylation of a substrate (e.g., autokinase activity) or endoribonuclease activity on a substrate e.g., XBP-1 mRNA. In another embodiment, an IRE-1 activity is an indirect activity, such as a downstream event brought about by interaction of the IRE-1 protein with a IRE-1 target or substrate. As IRE-1 is in a signal transduction pathway involving XBP-1, modulation of IRE-1 modulates a molecule in a signal transduction pathway involving XBP-1. Modulators which modulate an XBP-1 biological activity indirectly modulate expression and/or activity of a molecule in a signal transduction pathway involving XBP-1, e.g., IRE-1, PERK, eIF2α, or ATF6α.

As used herein, a “substrate” or “target molecule” or “binding partner” is a molecule with which a protein binds or interacts in nature, such that protein's function (e.g., modulation of activation of the UPR, plasma cell differentiation, IL-6 production, immunoglobulin production or apoptosis in the case of XBP-1) is achieved. For example, a target molecule can be a protein or a nucleic acid molecule. Exemplary target molecules of the invention include proteins in the same signaling pathway as the XBP-1 protein, e.g., proteins which can function upstream (including both stimulators and inhibitors of activity) or downstream of the XBP-1 protein in a pathway involving regulation of, for example, modulation of the UPR, modulation of cellular differentiation, modulation of IL-6 production, modulation of immunoglobulin production, modulation of the proteasome pathway, modulation of protein folding and transport, modulation of terminal B cell differentiation, and modulation of apoptosis. Exemplary XBP-1 target molecules include IRE-1, ATF6α, XBP-1 itself (as the molecule forms homodimers) cfos (which can form heterodimers with XBP-1) as well as the regulatory regions of genes regulated by XBP-1. Exemplary IRE-1 target molecules include XBP-1 and IRE-1 itself (as the molecule can form homodimers).

As used herein, the term “signal transduction pathway” includes the means by which a cell converts an extracellular influence or signal (e.g., a signal transduced by a receptor on the surface of a cell, such as a cytokine receptor or an antigen receptor) into a cellular response (e.g., modulation of gene transcription). Exemplary signal transduction pathways include the JAK1/STAT-1 pathway (Leonard, W. 2001. Int. J. Hematol. 73:271) and the TGF-β pathway (Attisano and Wrana. 2002. Science. 296:1646) A “signal transduction pathway involving XBP-1” is one in which XBP-1 is a signaling molecule which relays signals.

The subject methods can employ various target molecules. For example, an one embodiment, the subject methods can employ XBP-1. In another embodiment, the subject methods can employ at least one other molecule in an XBP-1 signaling pathway, e.g., a molecule either upstream or downstream of XBP-1. For example, in one embodiment, the subject methods can employ IRE-1. In another embodiment, the subject methods can employ ATF6α or PERK.

As used herein, the term “chaperone gene” is includes genes that are induced as a result of the activation of the UPR or the EOR. The chaperone genes include, for example, members of the family of Glucose Regulated Proteins (GRPs) such as GRP78 (BiP) and GRP94 (endoplasmin), as well as other chaperones such as calreticulin, protein disulfide isomerase, and ERp72. The upregulation of chaperone genes helps accommodate the increased demand for the folding capacity within the ER.

As used herein, the term “gene whose transcription is regulated by XBP-1”, includes genes having a regulatory region regulated by XBP-1. Such genes can be positively or negatively regulated by XBP-1. The term also includes genes which are indirectly regulated by XBP-1, e.g., are regulated by molecule in a signaling pathway in which XBP-1 is involved. Exemplary genes directly regulated by XBP-1 include, for example, genes such as ERdj4 (e.g., NM_{—}012328 [gi:9558754]), p58^{ipk} (e.g., XM_{—}209778 [gi:2749842] or NM_{—}006260 [gi:24234721]), EDEM (e.g., NM_{—}014674 [gi:7662001]), PDI-P5 (e.g., NC_{—}003284 [gi:32566600]), RAMP4 (e.g., AF136975 [gi:12239332]), HEDJ (e.g., AF228505 [gi: 7385134]), BiP (e.g., X87949 [gi: 1143491]), ATF6α (e.g., NM_{—}007348 [gi:6671584], XBP-1 (e.g., NM_{—}005080 [gi:14110394]), Armet (e.g., NM_{—}006010 [gi:51743920]) and/or DNAJB9 (which encodes mDj7) e.g., (NM_{—}012328 [gi:9558754]), the MHC class II genes (various MHC class II gene sequences are known in the art) and the IL-6 gene (e.g., MN_{—}000600 [gi 10834983]).

As used herein the term “apoptosis” includes programmed cell death which can be characterized using techniques which are known in the art. Apoptotic cell death can be characterized, e.g., by cell shrinkage, membrane blebbing and chromatin condensation culminating in cell fragmentation. Cells undergoing apoptosis also display a characteristic pattern of internucleosomal DNA cleavage. As used herein, the term “modulates apoptosis” includes either up regulation or down regulation of apoptosis in a cell.

As used herein, the term “cellular differentiation” includes the process by which the developmental potential of cells is restricted and they acquire specific developmental fates. Differentiated cells are recognizably different from other cell types.

As used herein, the term “plasma cell differentiation”, or “terminal B cell differentiation” refers to the process wherein B cells, which start their life in the bone marrow as pre-B cells, differentiate into plasma cells. Pre-B cells do not have antibody on their surface. A maturation step occurs which involves gene rearrangement of the immunoglobulin genes in the B cell and results in surface immunoglobulin (slg) being made and transported to the surface of the cell. The B cell with surface IgM and IgD becomes a mature but “naive” cell (since it has yet to see antigen). An encounter with antigen, along with help from T cell cytokines, stimulates B cell activation and terminal differentiation into a plasma cell. The differentiation of B cells into plasma cells occurs as the cell continues to divide in the presence of cytokines.

As used herein, the term “contacting” (i.e., contacting a cell e.g. a cell, with a compound) includes incubating the compound and the cell together in vitro (e.g., adding the compound to cells in culture) as well as administering the compound to a subject such that the compound and cells of the subject are contacted in vivo. The term “contacting” does not include exposure of cells to an XBP-1 modulator that may occur naturally in a subject (i.e., exposure that may occur as a result of a natural physiological process).

As used herein, the term “test compound” refers to a compound that has not previously been identified as, or recognized to be, a modulator of the activity being tested. The term “library of test compounds” refers to a panel comprising a multiplicity of test compounds.

As used herein, the term “dominant negative XBP-1 protein” includes XBP-1 molecules (e.g., portions or variants thereof) that compete with native (i.e., naturally occurring wild-type) XBP-1 molecules, but which do not have XBP-1 activity. Such molecules effectively decrease XBP-1 activity in a cell. As used herein, “dominant negative XBP-1 protein” refers to a modified form of XBP-1 which is a potent inhibitor of XBP-1 activity. Exemplary dominant negative inhibitors are described herein and lack a transactivation domain but retain the leucine zipper motif the N-terminal 188 or 136 amino acids of the spliced form of the XBP-1 protein, e.g., consist of the N-terminal 188 or 136 amino acids of the spliced form of the XBP-1 protein.

As used herein, the term “indicator composition” refers to a composition that includes a protein of interest (e.g., XBP-1 or a molecule in a signal transduction pathway involving XBP-1), for example, a cell that naturally expresses the protein, a cell that has been engineered to express the protein by introducing an expression vector encoding the protein into the cell, or a cell free composition that contains the protein (e.g., purified naturally-occurring protein or recombinantly-engineered protein).

As used herein, the term “cell” includes prokaryotic and eukaryotic cells. In one embodiment, a cell of the invention is a bacterial cell. In another embodiment, a cell of the invention is a fungal cell, such as a yeast cell. In another embodiment, a cell of the invention is a vertebrate cell, e.g., an avian or mammalian cell. In a preferred embodiment, a cell of the invention is a murine or human cell.

As used herein, the term “engineered” (as in an engineered cell) refers to a cell into which a nucleic acid molecule e.g., encoding an XBP-1 protein (e.g., a spliced and/or unspliced form of XBP-1) has been introduced.

As used herein, the term “cell free composition” refers to an isolated composition, which does not contain intact cells. Examples of cell free compositions include cell extracts and compositions containing isolated proteins.

As used herein, the term “reporter gene” refers to any gene that expresses a detectable gene product, e.g., RNA or protein. Preferred reporter genes are those that are readily detectable. The reporter gene can also be included in a construct in the form of a fusion gene with a gene that includes desired transcriptional regulatory sequences or exhibits other desirable properties. Examples of reporter genes include, but are not limited to CAT (chloramphenicol acetyl transferase) (Alton and Vapnek (1979), Nature 282: 864-869) luciferase, and other enzyme detection systems, such as beta-galactosidase; firefly luciferase (deWet et al. (1987), Mol. Cell. Biol. 7:725-737); bacterial luciferase (Engebrecht and Silverman (1984), PNAS 1: 4154-4158; Baldwin et al. (1984), Biochemistry 23: 3663-3667); alkaline phosphatase (Toh et al. (1989) Eur. J. Biochem. 182: 231-238, Hall et al. (1983) J. Mol. Appl. Gen. 2: 101), human placental secreted alkaline phosphatase (Cullen and Malim (1992) Methods in Enzymol. 216:362-368) and green fluorescent protein (U.S. Pat. No. 5,491,084; WO 96/23898).

As used herein, the term “XBP-1-responsive element” refers to a DNA sequence that is directly or indirectly regulated by the activity of the XBP-1 (whereby activity of XBP-1 can be monitored, for example, via transcription of a reporter gene).

As used herein, the term “cells deficient in XBP-1” includes cells of a subject that are naturally deficient in XBP-1, as wells as cells of a non-human XBP-1 deficient animal, e.g., a mouse, that have been altered such that they are deficient in XBP-1. The term “cells deficient in XBP-1” is also intended to include cells isolated from a non-human XBP-1 deficient animal or a subject that are cultured in vitro.

As used herein, the term “non-human XBP-1 deficient animal” refers to a non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal, such that the endogenous XBP-1 gene is altered, thereby leading to either no production of XBP-1 or production of a mutant form of XBP-1 having deficient XBP-1 activity. Preferably, the activity of XBP-1 is entirely blocked, although partial inhibition of XBP-1 activity in the animal is also encompassed. The term “non-human XBP-1 deficient animal” is also intended to encompass chimeric animals (e.g., mice) produced using a blastocyst complementation system, such as the RAG-2 blastocyst complementation system, in which a particular organ or organs (e.g., the lymphoid organs) arise from embryonic stem (ES) cells with homozygous mutations of the XBP-1 gene.

In one embodiment, small molecules can be used as test compounds. The term “small molecule” is a term of the art and includes molecules that are less than about 7500, less than about 5000, less than about 1000 molecular weight or less than about 500 molecular weight. In one embodiment, small molecules do not exclusively comprise peptide bonds. In another embodiment, small molecules are not oligomeric. Exemplary small molecule compounds which can be screened for activity include, but are not limited to, peptides, peptidomimetics, nucleic acids, carbohydrates, small organic molecules (e.g., Cane et al. 1998. Science 282:63), and natural product extract libraries. In another embodiment, the compounds are small, organic non-peptidic compounds. In a further embodiment, a small molecule is not biosynthetic. For example, a small molecule is preferably not itself the product of transcription or translation.

Various aspects of the present invention are described in further detail in the following subsections.

II. Screening Assays:

In one embodiment, the invention provides methods (also referred to herein as “screening assays”) for identifying modulators, i.e., candidate or test compounds or agents (e.g., enzymes, peptides, peptidomimetics, small molecules, ribozymes, or antisense or siRNA molecules) which bind, e.g., to XBP-1 or a molecule in a signaling pathway involving XBP-1 (e.g., IRE-1, or ATF6α proteins); have a stimulatory or inhibitory effect on the expression, processing (e.g., splicing), post-translational modification (e.g., glycosylation, ubiquitination, or phosphorylation, or activity of XBP-1) or a molecule in a signal transduction pathway involving XBP-1. For example, XBP-1, IRE-1, PERK, and ATF6α function in a signal transduction pathway involving XBP-1. Therefore, any of these molecules can be used in the subject screening assays. Although the specific embodiments described below in this section and in other sections may list XBP-1, IRE-1, ATF6α, and/or PERK as examples, other molecules in a signal transduction pathway involving XBP-1 can also be used in the subject screening assays.

In one embodiment, the ability of a compound to directly modulate the expression, processing (e.g., splicing), post-translational modification (e.g., glycosylation, ubiquitination, or phosphorylation), or activity of XBP-1 is measured in a screening assay of the invention.

In one embodiment, the ability of a compound to modulate the expression, processing (e.g., splicing), post-translational modification (e.g., glycosylation, ubiquitination, or phosphorylation), or activity of XBP-1 is measured in a cell that expresses ATF6α. In another embodiment, an agent is identified as one that modulates a biological activity of XBP-1 (e.g., modulates the UPR) even though it does not modulate expression, post-translational modification, and/or activity of ATF6α. In one embodiment of the invention, a compound is identified as one that modulates a biological activity of XBP-1 (e.g., modulates the UPR) even though its ability to modulate ATF6α is not tested. In one embodiment of the invention, the ability of a compound to modulate a biological activity of XBP-1 that is not dependent upon ATF6α is measured. In another embodiment, ATF6α is not used in a screening assay of the invention.

In one embodiment of the invention, the ability of a compound to modulate XBP-1 (or a molecule from a signal transduction pathway involving XBP-1) without inhibiting the 26S proteasome can be tested. In one embodiment of the invention, the ability of a compound to modulate XBP-1 (or a molecule from a signal transduction pathway involving XBP-1) without substantially modulating the NF-KB pathway can be tested.

The indicator composition can be a cell that expresses the XBP-1 protein or a molecule in a signal transduction pathway involving XBP-1, for example, a cell that naturally expresses or, more preferably, a cell that has been engineered to express the protein by introducing into the cell an expression vector encoding the protein. Preferably, the cell is a mammalian cell, e.g., a human cell. In one embodiment, the cell is a B cell. Alternatively, the indicator composition can be a cell-free composition that includes the protein (e.g., a cell extract or a composition that includes e.g., either purified natural or recombinant protein). In another embodiment, the cell is a secretory cell. In another embodiment, the cell is under ER stress. In yet another embodiment, the cell expresses ATF6α and PERK.

Compounds identified using the assays described herein can be useful for treating disorders associated with aberrant expression, processing, post-translational modification, or activity of XBP-1 or a molecule in a signaling pathway involving XBP-1 or a disorder involving XBP-1 e.g., aberrant activation of the UPR, aberrant cellular differentiation, aberrant IL-6 production, aberrant immunoglobulin production, aberrant activation of the proteasome pathway, aberrant protein folding and transport, aberrant terminal B cell differentiation, or aberrant cell proliferation (e.g., cells inappropriately undergoing apoptosis (e.g., in a neurodegenerative disorder or immunodeficiency disorder) or cells proliferating uncontrollably (e.g., cancer cells)). Conditions that can benefit from modulation of a signal transduction pathway involving XBP-1 include autoimmune disorders as well as malignancies and immunodeficiency disorders. Compounds which modulate XBP-1 expression and/or activity can also be used to modulate the immune response. In addition, XBP-1 or a molecule in a signal transduction pathway involving XBP-1 can be modulated to modulate protein folding and transport in normal cells, e.g., to increase expression, production, or secretion of a commercially valuable protein, e.g., an immunoglobulin.

The subject screening assays can be performed in the presence or absence of other agents. In one embodiment, the subject assays are performed in the presence of an agent that affects the unfolded protein response, e.g., tunicamycin, which evokes the UPR by inhibiting N-glycosylation, or thapsigargin. In another embodiment, the subject assays are performed in the presence of an agent that inhibits degradation of proteins by the ubiquitin-proteasome pathway (e.g., peptide aldehydes, such as MG132). In another embodiment, the screening assays can be performed in the presence or absence of a molecule that enhances cell activation, e.g., anti-CD40.

In another aspect, the invention pertains to a combination of two or more of the assays described herein. For example, a modulating agent can be identified using a cell-based or a cell-free assay, and the ability of the agent to modulate the activity of XBP-1 or a molecule in a signal transduction pathway involving XBP-1 can be confirmed in vivo, e.g., in an animal such as an animal model for multiple myeloma, neoplastic diseases, renal cell carcinoma or autoimmune diseases.

Moreover, a modulator of XBP-1 or a molecule in a signaling pathway involving XBP-1 identified as described herein (e.g., an enzyme, an antisense nucleic acid molecule, or a specific antibody, or a small molecule) can be used in an animal model to determine the efficacy, toxicity, or side effects of treatment with such a modulator. Alternatively, a modulator identified as described herein can be used in an animal model to determine the mechanism of action of such a modulator.

In another embodiment, it will be understood that similar screening assays can be used to identify compounds that indirectly modulate the activity and/or expression of XBP-1 e.g., by performing screening assays such as those described above using molecules with which XBP-1 interacts, e.g., molecules that act either upstream or downstream of XBP-1 (e.g., IRE-1, or ATF6α) in a signal transduction pathway.

The cell based and cell free assays of the invention are described in more detail below.

A. Cell Based Assays

The indicator compositions of the invention can be a cell that expresses an XBP-1 protein (or non-XBP-1 protein in the XBP-1 signaling pathway such as IRE-1 or ATF6α), for example, a cell that naturally expresses endogenous XBP-1, IRE-1, PERK or ATF6α or, more preferably, a cell that has been engineered to express an exogenous XBP-1, IRE-1, PERK, or ATF6α protein by introducing into the cell an expression vector encoding the protein. Alternatively, the indicator composition can be a cell-free composition that includes XBP-1 or a non-XBP-1 protein such as IRE-1 or ATF6α) (e.g., a cell extract from an XBP-1, IRE-1, or ATF6α-expressing cell or a composition that includes purified XBP-1, IRE-1, or ATF6α protein, either natural or recombinant protein).

Compounds that modulate expression and/or activity of XBP-1, or a non-XBP-1 protein that acts upstream or downstream of XBP-1 can be identified using various “read-outs.”

For example, an indicator cell can be transfected with an XBP-1 expression vector, incubated in the presence and in the absence of a test compound, and the effect of the compound on the expression of the molecule or on a biological response regulated by XBP-1 can be determined. In one embodiment, unspliced XBP-1 (e.g., capable of being spliced so that the cell will make both forms, or incapable of being spliced so the cell will make only the unspliced form) can be expressed in a cell. In another embodiment, spliced XBP-1 can be expressed in a cell. The biological activities of XBP-1 include activities determined in vivo, or in vitro, according to standard techniques. An XBP-1 activity can be a direct activity, such as an association with an XBP-1-target molecule (e.g., a nucleic acid molecule to which XBP-1 binds such as the transcriptional regulatory region of a chaperone gene) or a protein such as the IRE-1 or ATF6α protein. Alternatively, an XBP-1 activity is an indirect activity, such as a cellular signaling activity occurring downstream of the interaction of the XBP-1 protein with an XBP-1 target molecule or a biological effect occurring as a result of the signaling cascade triggered by that interaction. For example, biological activities of XBP-1 described herein include: modulation of the UPR, modulation of cellular differentiation, modulation of IL-6 production, modulation of immunoglobulin production, modulation of the proteasome pathway, modulation of protein folding and transport, modulation of terminal B cell differentiation, and modulation of apoptosis.

To determine whether a test compound modulates protein expression, in vitro transcriptional assays can be performed. In one example of such an assay, the full length promoter and enhancer of XBP-1 can be operably linked to a reporter gene such as chloramphenicol acetyltransferase (CAT) or luciferase and introduced into host cells. Other techniques are known in the art.

As used interchangeably herein, the terms “operably linked” and “operatively linked” are intended to mean that the nucleotide sequence is linked to a regulatory sequence in a manner which allows expression of the nucleotide sequence in a host cell (or by a cell extract). Regulatory sequences are art-recognized and can be selected to direct expression of the desired protein in an appropriate host cell. The term regulatory sequence is intended to include promoters, enhancers, polyadenylation signals and other expression control elements. Such regulatory sequences are known to those skilled in the art and are described in Goeddel, Gene Expression Technology: Methods in Enzymology 185, Academic Press, San Diego, Calif. (1990). It should be understood that the design of the expression vector may depend on such factors as the choice of the host cell to be transfected and/or the type and/or amount of protein desired to be expressed.

Exemplary constructs can include an XBP-1 target sequence TGGATGACGTGTACA (SEQ ID NO: 2) fused to the minimal promoter of the mouse RANTES gene (Clauss et al. Nucleic Acids Research 1996. 24:1855) or the ATF6/XBP-1 target TCGAGACAGGTGCTGACGTGGCGATTCC (SEQ ID NO: 3) and comprising −53/+45 of the cfos promoter (J. Biol. Chem. 275:27013) fused to a reporter gene. In one embodiment, multiple copies of the XBP-1 target sequence can be included.

A variety of reporter genes are known in the art and are suitable for use in the screening assays of the invention. Examples of suitable reporter genes include those which encode chloramphenicol acetyltransferase, beta-galactosidase, alkaline phosphatase or luciferase. Standard methods for measuring the activity of these gene products are known in the art.

A variety of cell types are suitable for use as an indicator cell in the screening assay. Preferably a cell line is used which expresses low levels of endogenous XBP-1, IRE-1, PERK, or ATF6α, and is then engineered to express recombinant XBP-1, IRE-1, PERK, or ATF6α. Cells for use in the subject assays include both eukaryotic and prokaryotic cells. For example, in one embodiment, a cell is a bacterial cell. In another embodiment, a cell is a fungal cell, such as a yeast cell. In another embodiment, a cell is a vertebrate cell, e.g., an avian cell or a mammalian cell (e.g., a murine cell, or a human cell).

In one embodiment, the level of expression of the reporter gene in the indicator cell in the presence of the test compound is higher than the level of expression of the reporter gene in the indicator cell in the absence of the test compound and the test compound is identified as a compound that stimulates the expression of XBP-1, IRE-1, or ATF6α. In another embodiment, the level of expression of the reporter gene in the indicator cell in the presence of the test compound is lower than the level of expression of the reporter gene in the indicator cell in the absence of the test compound and the test compound is identified as a compound that inhibits the expression of XBP-1, IRE-1, or ATF6α.

In one embodiment, the invention provides methods for identifying compounds that modulate cellular responses in which XBP-1 is involved. For example, in one embodiment, modulation of the UPR or ER stress can be determined. Transcription of genes encoding molecular chaperones and folding enzymes in the endoplasmic reticulum (ER) is induced by accumulation of unfolded proteins in the ER. This intracellular signaling, known as the unfolded protein response (UPR), is mediated by the cis-acting ER stress response element (ERSE) in mammals. In addition to ER chaperones, the mammalian transcription factor CHOP (also called GADD 153) is induced by ER stress. XBP-1 (also called TREB5) is also induced by ER stress and the induction of CHOP and XBP-1 is mediated by ERSE. The ERSE consensus sequence is CCAAT-N(9)-CCACG (SEQ ID NO: 4). As the general transcription factor NF-Y (also known as CBF) binds to CCAAT, CCACG is considered to provide specificity in the mammalian UPR. The basic leucine zipper protein ATF6 isolated as a CCACG -binding protein is synthesized as a transmembrane protein in the ER, and ER stress-induced proteolysis produces a soluble form of ATF6 that translocates into the nucleus.

Modulation of the UPR can be measured by, for example, measuring the changes in the endogenous levels of mRNA and the transcription or production of proteins such as ERdj4, p58^{ipk}, EDEM, PDI-P5, RAMP4, HEDJ, BiP, ATF6α, XBP-1, Armet and DNAJB9 or folding catalysts using routine ELISA, Northern and Western blotting techniques. In addition, the attenuation of translation associated with the UPR can be measured, e.g., by measuring protein production (Ruegsegger et al. 2001. Cell 107:103). Preferred proteins for detection are expressed on the cell surface or are secreted. In another embodiment, the phosphorylation of eukaryotic initiation factor 2 can be measured. In another embodiment, the accumulation of aggregated, misfolded, or damaged proteins in a cell can be monitored (Welch, W. J. 1992 Physiol. Rev. 72:1063; Gething and Sambrook. 1992. Nature. 355:33; Kuznetsov etal. 1997. J. Biol. Chem. 272:3057).

In one embodiment differentiation of cells can be used as an indicator of modulation of XBP-1 or a signal transduction pathway involving XBP-1. Cell differentiation can be monitored directly (e.g. by microscopic examination of the cells for monitoring cell differentiation), or indirectly, e.g., by monitoring one or more markers of cell differentiation (e.g., an increase in mRNA for a gene product associated with cell differentiation, or the secretion of a gene product associated with cell differentiation, such as the secretion of a protein (e.g., the secretion of immunoglobulin by differentiated plasma cells) or the expression of a cell surface marker (such as Syndecan expression by plasma cells) Reimold et al. 2001. Nature 412:300). Standard methods for detecting mRNA of interest, such as reverse transcription-polymerase chain reaction (RT-PCR) and Northern blotting, are known in the art. Standard methods for detecting protein secretion in culture supernatants, such as enzyme linked immunosorbent assays (ELISA), are also known in the art. Proteins can also be detected using antibodies, e.g., in an immunoprecipitation reaction or for staining and FACS analysis.

In one embodiment, the ability of a compound to induce terminal B cell differentiation can be determined. As described herein, terminal B cell differentiation can be measured in a variety of ways. Cells can be examined microscopically for the presence of the elaborate ER system characteristic of plasma cells. The secretion of immunoglobulin is also hallmark of plasma cell differentiation. Alternatively, the expression of a cell surface marker can be detected, e.g., surface IgM or Syndecan.

In one embodiment, the ability of a compound to modulate IL-6 production can be determined. Production of IL-6 can be monitored, for example, using Northern or Western blotting. IL-6 can also be detected using an ELISA assay or in a bioassay, e.g., employing cells which are responsive to IL-6 (e.g., cells which proliferate in response to the cytokine or which survive in the presence of the cytokine), such as plasma cells or multiple myeloma cells using standard techniques.

In another embodiment, the ability of a compound to modulate the proteasome pathway of a cell can be determined using any of a number of art-recognized techniques. For example, in one embodiment, the half life of normally short-lived regulatory proteins (e.g., NF-kB, cyclins, oncogenic products or tumor suppressors) can be measured to measure the degradation capacity of the proteasome. In another embodiment, the presentation of antigen in the context of MHC molecules on the surface of cells can be measured (e.g., in an in vitro assay of T cell activation) as proteasome degradation of antigen is important in antigen processing and presentation. In another embodiment, threonine protease activity associated with the proteasome can be measured. Agents that modulate the proteasome pathway will affect the normal degradation of these proteins. In another embodiment, the modulation of the proteasome pathway can be measured indirectly by measuring the ratio of spliced to unspliced XBP-1 or the ratio of unspliced to spliced XBP-1. As described in the instant examples, inhibition of the proteasome pathway, e.g., by the inhibitor MG-132, leads to an increase in the level of unspliced XBP-1 as compared to spliced XBP-1. The levels of these different forms of XBP-1 can be measured using various techniques described herein (e.g., Western blotting or PCR) or known in the art and a ratio determined. In one embodiment, the ability of a compound to modulate protein folding or transport can be determined. The expression of a protein on the surface of a cell or the secretion of a secreted protein can be measured as indicators of protein folding and transport. Protein expression on a cell can be measured, e.g., using FACS analysis, surface iodination, immunoprecipitation from membrane preparations. Protein secretion can be measured, for example, by measuring the level of protein in a supernatant from cultured cells. The production of any secreted protein can be measured in this manner. The protein to be measured can be endogenous or exogenous to the cell. In preferred embodiment, the protein is selected from the group consisting of: α-fetoprotein, α1-antitrypsin, albumin, luciferase and immunoglobulins. The production of proteins can be measured using standard techniques in the art.

In another embodiment, the ability of a compound to modulate apoptosis, e.g., modulate apoptosis by disrupting the UPR, can be determined. In one embodiment, the ability of a compound to modulate apoptosis in a secretory cell or a cell under ER stress is determined. Apoptosis can be measured in the presence or the absence of Fas-mediated signals. In one embodiment, cytochrome C release from mitochondria during cell apoptosis can be detected, e.g., plasma cell apoptosis (as described in, for example, Bossy-Wetzel E. et al. (2000) Methods in Enzymol. 322:235-42). Other exemplary assays include: cytofluorometric quantitation of nuclear apoptosis induced in a cell-free system (as described in, for example, Lorenzo H. K. et al. (2000) Methods in Enzymol. 322:198-201); apoptotic nuclease assays (as described in, for example, Hughes F. M. (2000) Methods in Enzymol. 322:47-62); analysis of apoptotic cells, e.g., apoptotic plasma cells, by flow and laser scanning cytometry (as described in, for example, Darzynkiewicz Z. et al. (2000) Methods in Enzymol. 322:18-39); detection of apoptosis by annexin V labeling (as described in, for example, Bossy-Wetzel E. et al. (2000) Methods in Enzymol. 322:15-18); transient transfection assays for cell death genes (as described in, for example, Miura M. et al. (2000) Methods in Enzymol. 322:480-92); and assays that detect DNA cleavage in apoptotic cells, e.g., apoptotic plasma cells (as described in, for example, Kauffman S. H. et al. (2000) Methods in Enzymol. 322:3-15). Apoptosis can also be measured by propidium iodide staining or by TUNEL assay. In another embodiment, the transcription of genes associated with a cell signaling pathway involved in apoptosis (e.g., JNK) can be detected using standard methods.

In another embodiment, mitochondrial inner membrane permeabilization can be measured in intact cells by loading the cytosol or the mitochondrial matrix with a die that does not normally cross the inner membrane, e.g., calcein (Bernardi et al. 1999. Eur. J. Biochem. 264:687; Lemasters, J., J. et al. 1998. Biochem. Biophys. Acta 1366:177. In another embodiment, mitochondrial inner membrane permeabilization can be assessed, e.g., by determining a change in the mitochondrial inner membrane potential (ΔΨm). For example, cells can be incubated with lipophilic cationic fluorochromes such as DiOC6 (Gross et al. 1999. Genes Dev. 13:1988) (3,3′dihexyloxacarbocyanine iodide) or JC-1(5,5′,6,6′-tetrachloro-1,1′,3,3′-tetraethylbenzimidazolylcarbocyanine iodide). These dyes accumulate in the mitochondrial matrix, driven by the Ψm. Dissipation results in a reduction of the fluorescence intensity (e.g., for DiOC6 (Gross et al. 1999. Genes Dev. 13:1988) or a shift in the emission spectrum of the dye. These changes can be measured by cytofluorometry or microscopy.

In yet another embodiment, the ability of a compound to modulate translocation of spliced XBP-1 to the nucleus can be determined. Translocation of spliced XBP-1 to the nucleus can be measured, e.g., by nuclear translocation assays in which the emission of two or more fluorescently-labeled species is detected simultaneously. For example, the cell nucleus can be labeled with a known fluorophore specific for DNA, such as Hoechst 33342. The spliced XBP-1 protein can be labeled by a variety of methods, including expression as a fusion with GFP or contacting the sample with a fluorescently-labeled antibody specific spliced XBP-1. The amount spliced XBP-1 that translocates to the nucleus can be determined by determining the amount of a first fluorescently-labeled species, i.e., the nucleus, that is distributed in a correlated or anti-correlated manner with respect to a second fluorescently-labeled species, i.e., spliced XBP-1, as described in U.S. Pat. No. 6,400,487, the contents of which are hereby incorporated by reference.

The ability of the test compound to modulate XBP-1 (or a molecule in a signal transduction pathway involving to XBP-1) binding to a substrate or target molecule (e.g., IRE-1 or ATF6α in the case of XBP-1) can also be determined. Determining the ability of the test compound to modulate XBP-1 (or e.g., IRE-1, or ATF6α) binding to a target molecule (e.g., a binding partner such as a substrate) can be accomplished, for example, by coupling the target molecule with a radioisotope or enzymatic label such that binding of the target molecule to XBP-1 or a molecule in a signal transduction pathway involving XBP-1 can be determined by detecting the labeled XBP-1 (or e.g., IRE-1 or ATF6α) target molecule in a complex. Alternatively, XBP-1(or e.g., IRE-1 or ATF6α) could be coupled with a radioisotope or enzymatic label to monitor the ability of a test compound to modulate XBP-1(or e.g., IRE-1 or ATF6α) binding to a target molecule in a complex. Determining the ability of the test compound to bind to XBP-1(or e.g., IRE-1 or ATF6α) can be accomplished, for example, by coupling the compound with a radioisotope or enzymatic label such that binding of the compound to XBP-1 (or e.g., IRE-1 or ATF6α) can be determined by detecting the labeled compound in a complex. For example, targets can be labeled with ¹²⁵I, ³⁵S, ¹⁴C, or ³H, either directly or indirectly, and the radioisotope detected by direct counting of radioemmission or by scintillation counting. Alternatively, compounds can be labeled, e.g., with, for example, horseradish peroxidase, alkaline phosphatase, or luciferase, and the enzymatic label detected by determination of conversion of an appropriate substrate to product.

In another embodiment, the ability of XBP-1 or a molecule in a signal transduction pathway involving XBP-1 to be acted on by an enzyme or to act on a substrate can be measured. For example, in one embodiment, the effect of a compound on the phosphorylation of IRE-1, the ability of IRE-1 to process XBP-1, the ability of PERK to phosphorylate a substrate can be measured using techniques that are known in the art.

It is also within the scope of this invention to determine the ability of a compound to interact with XBP-1 or a molecule in a signal transduction pathway involving XBP-1 without the labeling of any of the interactants. For example, a microphysiometer can be used to detect the interaction of a compound with XBP-1, IRE-1, or ATF6α without the labeling of either the compound or the XBP-1, IRE-1, or ATF6α (McConnell, H. M. et al. (1992) Science 257:1906-1912). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and XBP-1, IRE-1, or ATF6α.

Exemplary target molecules of XBP-1 include: XBP-1-responsive elements, for example, upstream regulatory regions from genes such as α-1 antitrypsin, α-fetoprotein, HLA DRα, as well as the 21 base pair repeat enhancer of the HTLV-1 LTR. An example of an XBP-1-responsive reporter construct is the HLA DRα-CAT construct described in Ono, S. J. et al. (1991) Proc. Natl. Acad. Sci. USA 88:4309-4312. Other examples can include regulatory regions of the chaperone genes such as members of the family of Glucose Regulated Proteins (GRPs) such as GRP78 (BiP) and GRP94 (endoplasmin), as well as other chaperones such as calreticulin, protein disulfide isomerase, and ERp72. XBP-1 targets are taught, e.g. in Clauss et al. Nucleic Acids Research 1996. 24:1855 also include CRE and TRE sequences

In another embodiment, a different (i.e., non-XBP-1) molecule acting in a pathway involving XBP-1 that acts upstream (e.g., IRE-1) or downstream (e.g., ATF6α or cochaperone proteins that activate ER resident HspTO proteins, such as p58^{IPK}) of XBP-1 can be included in an indicator composition for use in a screening assay. Compounds identified in a screening assay employing such a molecule would also be useful in modulating XBP-1 activity, albeit indirectly. IRE-1 is one exemplary IRE-1 substrate (e.g., the autophosphorylation of IRE-1). In another embodiment, the endoribonuclease activity of IRE-1 can be measured, e.g., by detecting the splicing of XBP-1 using techniques that are known in the art. The activity of IRE-1 can also be measured by measuring the modulation of biological activity associated with XBP-1.

The cells used in the instant assays can be eukaryotic or prokaryotic in origin. For example, in one embodiment, the cell is a bacterial cell. In another embodiment, the cell is a fungal cell, e.g., a yeast cell. In another embodiment, the cell is a vertebrate cell, e.g., an avian or a mammalian cell. In a preferred embodiment, the cell is a human cell.

The cells of the invention can express endogenous XBP-1 or another protein in a signaling pathway involving XBP-1 or can be engineered to do so. For example, a cell that has been engineered to express the XBP-1 protein and/or a non XBP-1 protein which acts upstream or downstream of XBP-1 can be produced by introducing into the cell an expression vector encoding the protein.

In one embodiment, to specifically assess the role of agents that modulate the expression and/or activity of unspliced or spliced XBP-1 protein, retroviral gene transduction of cells deficient in XBP-1 with spliced XBP-1 or a form of XBP-1 which cannot be spliced can be performed. For example, a construct such as that described in the instant examples in which mutations at in the loop structure of XBP-1 (e.g., positions −1 and +3 in the loop structure of XBP-1) can be generated. Expression of this construct in cells results in production of the unspliced form of XBP-1 only. Using such constructs, the ability of a compound to modulate a particular form of XBP-1 can be detected. In one embodiment, a compound modulates one form of XBP-1, e.g., spliced XBP-1, without modulating the other form, e.g., unspliced XBP-1.

Recombinant expression vectors that can be used for expression of XBP-1 or a molecule in a signal transduction pathway involving XBP-1 (e.g., a protein which acts upstream or downstream of XBP-1) or a molecule in a signal transduction pathway involving XBP-1 in the indicator cell are known in the art. For example, the XBP-1, IRE-1, or ATF6α cDNA is first introduced into a recombinant expression vector using standard molecular biology techniques. A cDNA can be obtained, for example, by amplification using the polymerase chain reaction (PCR) or by screening an appropriate cDNA library. The nucleotide sequences of cDNAs for XBP-1 or a molecule in a signal transduction pathway involving XBP-1 (e.g., human, murine and yeast) are known in the art and can be used for the design of PCR primers that allow for amplification of a cDNA by standard PCR methods or for the design of a hybridization probe that can be used to screen a cDNA library using standard hybridization methods. The nucleotide and predicted amino acid sequences of a mammalian XBP-1 cDNA are disclosed in Liou, H-C. et. al. (1990) Science 247:1581-1584, Yoshimura, T. et al. (1990) EMBO J. 9:2537-2542, and Kishimoto T. et al., (1996) Biochem. Biophys. Res. Commun. 223:746-751. The nucleotide sequences of human, mouse, C. elegans and yeast IRE-1 are disclosed, for example in Calfon et al. (2002) Nature 415:92-96.

Following isolation or amplification of a cDNA molecule encoding XBP-1 or a non-XBP-1 molecule in a signal transduction pathway involving XBP-1 the DNA fragment is introduced into an expression vector. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” or simply “expression vectors”. In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid molecule in a form suitable for expression of the nucleic acid in a host cell, which means that the recombinant expression vectors include one or more regulatory sequences, selected on the basis of the host cells to be used for expression and the level of expression desired, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to includes promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell, those which direct expression of the nucleotide sequence only in certain host cells (e.g., tissue-specific regulatory sequences) or those which direct expression of the nucleotide sequence only under certain conditions (e.g., inducible regulatory sequences).

When used in mammalian cells, the expression vector's control functions are often provided by viral regulatory elements. For example, commonly used promoters are derived from polyoma virus, adenovirus, cytomegalovirus and Simian Virus 40. Non-limiting examples of mammalian expression vectors include pCDM8 (Seed, B., (1987) Nature 329:840) and pMT2PC (Kaufman et al. (1987), EMBO J. 6: 187-195). A variety of mammalian expression vectors carrying different regulatory sequences are commercially available. For constitutive expression of the nucleic acid in a mammalian host cell, a preferred regulatory element is the cytomegalovirus promoter/enhancer. Moreover, inducible regulatory systems for use in mammalian cells are known in the art, for example systems in which gene expression is regulated by heavy metal ions (see e.g., Mayo et al. (1982) Cell 29:99-108; Brinster et al. (1982) Nature 296:39-42; Searle et al. (1985) Mol. Cell. Biol. 5:1480-1489), heat shock (see e.g., Nouer et al. (1991) in Heat Shock Response, e.d. Nouer, L. , CRC, Boca Raton, Fla., pp167-220), hormones (see e.g., Lee et al. (1981) Nature 294:228-232; Hynes et al. (1981) Proc. Natl. Acad. Sci. USA 78:2038-2042; Klock et al. (1987) Nature 329:734-736; Israel & Kaufman (1989) Nucl. Acids Res. 17:2589-2604; and PCT Publication No. WO 93/23431), FK506-related molecules (see e.g., PCT Publication No. WO 94/18317) or tetracyclines (Gossen, M. and Bujard, H. (1992) Proc. Natl. Acad Sci. USA 89:5547-5551; Gossen, M. et al. (1995) Science 268:1766-1769; PCT Publication No. WO 94/29442; and PCT Publication No. WO 96/01313). Still further, many tissue-specific regulatory sequences are known in the art, including the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916) and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

Vector DNA can be introduced into mammalian cells via conventional transfection techniques. As used herein, the various forms of the term “transfection” are intended to refer to a variety of art-recognized techniques for introducing foreign nucleic acid (e.g., DNA) into mammalian host cells, including calcium phosphate co-precipitation, DEAE-dextran-mediated transfection, lipofection, or electroporation. Suitable methods for transfecting host cells can be found in Sambrook et al. (Molecular Cloning: A Laboratory Manual, 2nd Edition, Cold Spring Harbor Laboratory press (1989)), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on a separate vector from that encoding XBP-1 or, more preferably, on the same vector. Cells stably transfected with the introduced nucleic acid can be identified by drug selection (e.g., cells that have incorporated the selectable marker gene will survive, while the other cells die).

In one embodiment, within the expression vector coding sequences are operatively linked to regulatory sequences that allow for constitutive expression of the molecule in the indicator cell (e.g., viral regulatory sequences, such as a cytomegalovirus promoter/enhancer, can be used). Use of a recombinant expression vector that allows for constitutive expression of XBP-1 or a molecule in a signal transduction pathway involving XBP-1 in the indicator cell is preferrefed for identification of compounds that enhance or inhibit the activity of the molecule. In an alternative embodiment, within the expression vector the coding sequences are operatively linked to regulatory sequences of the endogenous gene for XBP-1 or a molecule in a signal transduction pathway involving XBP-1 (i.e., the promoter regulatory region derived from the endogenous gene). Use of a recombinant expression vector in which expression is controlled by the endogenous regulatory sequences is preferred for identification of compounds that enhance or inhibit the transcriptional expression of the molecule.

B. Assays Measuring Spliced v. Unspliced XBP-1

In another embodiment, the invention provides for screening assays to identify compounds which alter the ratio of spliced XBP-1 to unspliced XBP-1 or the ratio of unspliced XBP-1 to spliced XBP-1. Only the spliced form of XBP-1 mRNA activates gene transcription. Unspliced XBP-1 mRNA inhibits the activity of spliced XBP-1 mRNA. As explained above, human and murine XBP-1 mRNA contain an open reading frame (ORF1) encoding bZIP proteins of 261 and 267 amino acids, respectively. Both mRNA's also contain another ORF, ORF2, partially overlapping but not in frame with ORF1. ORF2 encodes 222 amino acids in both human and murine cells. Human and murine ORF1 and ORF2 in the XBP-1 mRNA share 75% and 89% identity respectively. In response to ER stress, XBP-1 MRNA is processed by the ER transmembrane endoribonuclease and kinase IRE-1 which excises an intron from XBP-1 mRNA. In murine and human cells, a 26 nucleotide intron is excised. Splicing out of 26 nucleotides in murine cells results in a frame shift at amino acid 165. This causes removal of the C-terminal 97 amino acids from the first open reading frame (ORF1) and addition of the 212 amino from ORF2 to the N-terminal 164 amino acids of ORF1 containing the b-ZIP domain. In mammalian cells, this splicing event results in the conversion of an approximately 267 amino acid unspliced XBP-1 protein to a 371 amino acid spliced XBP-1 protein. The spliced XBP-1 then translocates into the nucleus where it binds to its target sequences to induce their transcription.

Compounds that alter the ratio of unspliced to spliced XBP-1 or spliced to unspliced XBP-1 can be useful to modulate the biological activities of XBP-1, e.g., in modulation of the UPR, modulation of cellular differentiation, modulation of IL-6 production, modulation of immunoglobulin production, modulation of the proteasome pathway, modulation of protein folding and transport, modulation of terminal B cell differentiation, and modulation of apoptosis. The compounds can also be used to treat disorders that would benefit from modulation of XBP-1 expression and/or activity, e.g., autoimmune disorders, and malignancies.

The techniques for assessing the ratios of unspliced to spliced XBP-1 and spliced to unspliced XBP-1 are routine in the art. For example, the two forms can be distinguished based on their size, e.g., using northern blots or western blots. Because the spliced form of XBP-1 comprises an exon not found in the unspliced form, in another embodiment, antibodies that specifically recognize the spliced or unspliced form of XBP-1 can be developed using techniques well known in the art (Yoshida et al. 2001. Cell. 107:881). In addition, PCR can be used to distinguish spliced from unspliced XBP-1. For example, as described herein, primer sets can be used to amplify XBP-1 where the primers are derived from positions 410 and 580 of murine XBP-1, or corresponding positions in related XBP-1 molecules, in order to amplify the region that encompasses the splice junction. A fragment of 171 base pairs corresponds to unspliced XBP-1 mRNA. An additional band of 145 bp corresponds to the spliced form of XBP-1. The ratio of the different forms of XBP-1 can be determined using these or other art recognized methods.

C. Cell-Free Assays

In another embodiment, the indicator composition is a cell free composition. XBP-1 or a non-XBP-1 protein in a signal transduction pathway involving XBP-1 expressed by recombinant methods in a host cells or culture medium can be isolated from the host cells, or cell culture medium using standard methods for protein purification. For example, ion-exchange chromatography, gel filtration chromatography, ultrafiltration, electrophoresis, and immunoaffinity purification with antibodies can be used to produce a purified or semi-purified protein that can be used in a cell free composition. Alternatively, a lysate or an extract of cells expressing the protein of interest can be prepared for use as cell-free composition.

In one embodiment, compounds that specifically modulate XBP-1 activity or the activity of a molecule in a signal transduction pathway involving XBP-1 are identified based on their ability to modulate the interaction of XBP-1 (or e.g., IRE-1 or ATF6α) with a target molecule to which XBP-1 (or e.g., IRE-1 or ATF6α) binds. The target molecule can be a DNA molecule, e.g., an XBP-1-responsive element, such as the regulatory region of a chaperone gene) or a protein molecule. Suitable assays are known in the art that allow for the detection of protein-protein interactions (e.g., immunoprecipitations, two-hybrid assays and the like) or that allow for the detection of interactions between a DNA binding protein with a target DNA sequence (e.g., electrophoretic mobility shift assays, DNAse I footprinting assays and the like). By performing such assays in the presence and absence of test compounds, these assays can be used to identify compounds that modulate (e.g., inhibit or enhance) the interaction of XBP-1 with a target molecule.

In one embodiment, the amount of binding of XBP-1 or a molecule in a signal transduction pathway involving XBP-1 to the target molecule in the presence of the test compound is greater than the amount of binding of XBP-1(or e.g., IRE-1 or ATF6α) to the target molecule in the absence of the test compound, in which case the test compound is identified as a compound that enhances binding of XBP-1(or e.g., IRE-1 or ATF6α) to a target. In another embodiment, the amount of binding of the XBP-1 (or e.g., IRE-1 or ATF6α) to the target molecule in the presence of the test compound is less than the amount of binding of the XBP-1(or e.g., IRE-1 or ATF6α) to the target molecule in the absence of the test compound, in which case the test compound is identified as a compound that inhibits binding of XBP-1(or e.g., IRE-1 or ATF6α) to the target.

Binding of the test compound to XBP-1 or a molecule in a signal transduction pathway involving XBP-1 can be determined either directly or indirectly as described above. Determining the ability of XBP-1(or e.g., IRE-1 or ATF6α) protein to bind to a test compound can also be accomplished using a technology such as real-time Biomolecular Interaction Analysis (BIA) (Sjolander, S. and Urbaniczky, C. (1991) Anal. Chem. 63:2338-2345; Szabo et al. (1995) Curr. Opin. Struct. Biol. 5:699-705). As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In the methods of the invention for identifying test compounds that modulate an interaction between XBP-1 (or e.g., IRE-1 or ATF6α) protein and a target molecule, the complete XBP-1(or e.g., IRE-1 or ATF6α) protein can be used in the method, or, alternatively, only portions of the protein can be used. For example, an isolated XBP-1 b-ZIP structure (or a larger subregion of XBP-1 that includes the b-ZIP structure) can be used. In another example, a form of XBP-1 comprising the splice junction can be used (e.g., a portion including from about nucleotide 506 to about nucleotide 532). The degree of interaction between the protein and the target molecule can be determined, for example, by labeling one of the proteins with a detectable substance (e.g., a radiolabel), isolating the non-labeled protein and quantitating the amount of detectable substance that has become associated with the non-labeled protein. The assay can be used to identify test compounds that either stimulate or inhibit the interaction between the XBP-1(or e.g., IRE-1 or ATF6α) protein and a target molecule. A test compound that stimulates the interaction between the protein and a target molecule is identified based upon its ability to increase the degree of interaction between, e.g., spliced XBP-1 and a target molecule as compared to the degree of interaction in the absence of the test compound and such a compound would be expected to increase the activity of spliced XBP-1 in the cell. A test compound that inhibits the interaction between the protein and a target molecule is identified based upon its ability to decrease the degree of interaction between the protein and a target molecule as compared to the degree of interaction in the absence of the compound and such a compound would be expected to decrease spliced XBP-1 activity.

In one embodiment of the above assay methods of the present invention, it may be desirable to immobilize either XBP-1 (or a molecule in a signal transduction pathway involving XBP-1, e.g., IRE-1 or ATF6cc) or a respective target molecule for example, to facilitate separation of complexed from uncomplexed forms of one or both of the proteins, or to accommodate automation of the assay. Binding of a test compound to a XBP-1 or a molecule in a signal transduction pathway involving XBP-1, or interaction of an XBP-1 protein (or a molecule in a signal transduction pathway involving XBP-1, e.g., IRE-1 or ATF6α) with a target molecule in the presence and absence of a test compound, can be accomplished in any vessel suitable for containing the reactants. Examples of such vessels include microtitre plates, test tubes, and micro-centrifuge tubes. In one embodiment, a fusion protein can be provided in which a domain that allows one or both of the proteins to be bound to a matrix is added to one or more of the molecules. For example, glutathione-S-transferase fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or XBP-1 (or .g., IRE-1 or ATF6α) protein, and the mixture incubated under conditions conducive to complex formation (e.g., at physiological conditions for salt and pH). Following incubation, the beads or microtitre plate wells are washed to remove any unbound components, the matrix is immobilized in the case of beads, and complex formation is determined either directly or indirectly, for example, as described above. Alternatively, the complexes can be dissociated from the matrix, and the level of binding or activity determined using standard techniques.

Other techniques for immobilizing proteins on matrices can also be used in the screening assays of the invention. For example, either an XBP-1 protein or a molecule in a signal transduction pathway involving XBP-1, or a target molecule can be immobilized utilizing conjugation of biotin and streptavidin. Biotinylated protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies which are reactive with protein or target molecules but which do not interfere with binding of the protein to its target molecule can be derivatized to the wells of the plate, and unbound target or XBP-1 (or e.g., IRE-1 or ATF6α) protein is trapped in the wells by antibody conjugation. Methods for detecting such complexes, in addition to those described above for the GST-immobilized complexes, include inmmunodetection of complexes using antibodies reactive with XBP-1 or a molecule in a signal transduction pathway involving XBP-1 or target molecule, as well as enzyme-linked assays which rely on detecting an enzymatic activity associated with the XBP-1, IRE-1, or ATF6α protein or target molecule.

In yet another aspect of the invention, the XBP-1 protein(or .g., IRE-1 or ATF6α) or fragments thereof can be used as “bait proteins” e.g., in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with XBP-1 (“binding proteins” or “bp”) and are involved in XBP-1 activity. Such XBP-1-binding proteins are also likely to be involved in the propagation of signals by the XBP-1 proteins or XBP-1 targets such as, for example, downstream elements of an XBP-1-mediated signaling pathway. Alternatively, such XBP-1-binding proteins can be XBP-1 inhibitors.

The two-hybrid system is based on the modular nature of most transcription factors, which consist of separable DNA-binding and activation domains. Briefly, the assay utilizes two different DNA constructs. In one construct, the gene that codes for an XBP-1 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an XBP-1 dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the XBP-1 protein or a molecule in a signal transduction pathway involving XBP-1.

D. Assays Using Knock-Out Cells

In another embodiment, the invention provides methods for identifying compounds that modulate a biological effect of XBP-1 or a molecule in a signal transduction pathway involving XBP-1 using cells deficient in XBP-1(or e.g., IRE-1 or ATF6α). As described in the Examples, inhibition of XBP-1 activity (e.g., by disruption of the XBP-1 gene) in B cells results, e.g., in a deficiency of Ig production. Thus, cells deficient in XBP-1 or a molecule in a signal transduction pathway involving XBP-1 can be used identify agents that modulate a biological response regulated by XBP-1 by means other than modulating XBP-1 itself (i. e., compounds that “rescue” the XBP-1 deficient phenotype). Alternatively, a “conditional knock-out” system, in which the gene is rendered non-functional in a conditional manner, can be used to create deficient cells for use in screening assays. For example, a tetracycline-regulated system for conditional disruption of a gene as described in WO 94/29442 and U.S. Pat. No. 5,650,298 can be used to create cells, or animals from which cells can be isolated, be rendered deficient in XBP-1(or a molecule in a signal transduction pathway involving XBP-1 e.g., IRE-1 or ATF6α) in a controlled manner through modulation of the tetracycline concentration in contact with the cells. For assays relating to plasma cell differentiation, a similar conditional disruption approach can be used or, alternatively, the RAG-2 deficient blastocyst complementation system can be used to generate mice with lymphoid organs that arise from embryonic stem cells with homozygous mutations of the XBP-1(or e.g., IRE-1 or ATF6α) gene. Specific cell types, e.g., lymphoid cells (e.g., thymic, splenic and/or lymph node cells) or purified cells such as B cells from such animals can be used in screening assays.

In the screening method, cells deficient in XBP-1 or a molecule in a signal transduction pathway involving XBP-1 can be contacted with a test compound and a biological response regulated by XBP-1 or a molecule in a signal transduction pathway involving XBP-1 can be monitored. Modulation of the response in cells deficient in XBP-1 or a molecule in a signal transduction pathway involving XBP-1 (as compared to an appropriate control such as, for example, untreated cells or cells treated with a control agent) identifies a test compound as a modulator of the XBP-1(or e.g., IRE-1 or ATF6α) regulated response. In another embodiment, to specifically assess the role of agents that modulate unspliced or spliced XBP-1 protein, retroviral gene transduction of cells deficient in XBP-1, to express spliced XBP-1 or a form of XBP-1 which cannot be spliced can be performed. For example, a construct such as that described in the instant examples in which mutations at in the loop structure of XBP-1 (e.g., positions −1 and +3 in the loop structure of XBP-1) can be generated. Expression of this construct in cells results in production of the unspliced form of XBP-1 only. Using such constructs, the ability of a compound to modulate a particular form of XBP-1 can be detected. For example, in one embodiment, a compound modulates one form of XBP-1 without modulating the other form.

In one embodiment, the test compound is administered directly to a non-human knock out animal, preferably a mouse (e.g., a mouse in which the XBP gene or a gene in a signal transduction pathway involving XBP-1 is conditionally disrupted by means described above, or a chimeric mouse in which the lymphoid organs are deficient in XBP-1 or a molecule in a signal transduction pathway involving XBP-1 as described above), to identify a test compound that modulates the in vivo responses of cells deficient in XBP-1 (or e.g., IRE-1 or ATF6α). In another embodiment, cells deficient in XBP-1 (or e.g., IRE-1 or ATF6α) are isolated from the non-human XBP-1 or a molecule in a signal transduction pathway involving XBP-1 deficient animal, and contacted with the test compound ex vivo to identify a test compound that modulates a response regulated by XBP-1(or e.g., IRE-1 or ATF6α) in the cells

Cells deficient in XBP-1 or a molecule in a signal transduction pathway involving XBP-1 can be obtained from a non-human animals created to be deficient in XBP-1 or a molecule in a signal transduction pathway involving XBP-1 Preferred non-human animals include monkeys, dogs, cats, mice, rats, cows, horses, goats and sheep. In preferred embodiments, the deficient animal is a mouse. Mice deficient in XBP-1 or a molecule in a signal transduction pathway involving XBP-1 can be made using methods known in the art. Non-human animals deficient in a particular gene product typically are created by homologous recombination. Briefly, a vector is prepared which contains at least a portion of the gene into which a deletion, addition or substitution has been introduced to thereby alter, e.g., functionally disrupt, the endogenous XBP-1 (or e.g., IRE-1 or ATF6α gene). The gene preferably is a mouse gene. For example, a mouse XBP-1 gene can be isolated from a mouse genomic DNA library using the mouse XBP-1 cDNA as a probe. The mouse XBP-1 gene then can be used to construct a homologous recombination vector suitable for modulating an endogenous XBP-1 gene in the mouse genome. In a preferred embodiment, the vector is designed such that, upon homologous recombination, the endogenous gene is functionally disrupted (i.e., no longer encodes a functional protein; also referred to as a “knock out” vector).

Alternatively, the vector can be designed such that, upon homologous recombination, the endogenous gene is mutated or otherwise altered but still encodes functional protein (e.g., the upstream regulatory region can be altered to thereby alter the expression of the endogenous XBP-1 protein). In the homologous recombination vector, the altered portion of the gene is flanked at its 5′ and 3′ ends by additional nucleic acid of the gene to allow for homologous recombination to occur between the exogenous gene carried by the vector and an endogenous gene in an embryonic stem cell. The additional flanking nucleic acid is of sufficient length for successful homologous recombination with the endogenous gene. Typically, several kilobases of flanking DNA (both at the 5′ and 3′ ends) are included in the vector (see e.g., Thomas, K. R. and Capecchi, M. R. (1987) Cell 51:503 for a description of homologous recombination vectors). The vector is introduced into an embryonic stem cell line (e.g., by electroporation) and cells in which the introduced gene has homologously recombined with the endogenous gene are selected (see e.g., Li, E. et al. (1992) Cell 69:915). The selected cells are then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In another embodiment, retroviral transduction of donor bone marrow cells from both wild type and null mice can be performed, e.g., with the XBP-1 unspliced, DN or spliced constructs to reconstitute irradiated RAG recipients. This will result in the production of mice whose lymphoid cells express only unspliced, or only spliced XBP-1 protein, or which express a dominant negative version of XBP-1. B cells from these mice can then be tested for compounds that modulate a biological response regulated by XBP-1.

In one embodiment of the screening assay, compounds tested for their ability to modulate a biological response regulated by XBP-1 or a molecule in a signal transduction pathway involving XBP-1 are contacted with deficient cells by administering the test compound to a non-human deficient animal in vivo and evaluating the effect of the test compound on the response in the animal.

The test compound can be administered to a non-knock out animal as a pharmaceutical composition. Such compositions typically comprise the test compound and a pharmaceutically acceptable carrier. As used herein the term “pharmaceutically acceptable carrier” includes any and all solvents, dispersion media, coatings, antibacterial and antifungal compounds, isotonic and absorption delaying compounds, and the like, compatible with pharmaceutical administration. The use of such media and compounds for pharmaceutically active substances is well known in the art. Except insofar as any conventional media or compound is incompatible with the active compound, use thereof in the compositions is contemplated. Supplementary active compounds can also be incorporated into the compositions. Pharmaceutical compositions are described in more detail below.

In another embodiment, compounds that modulate a biological response regulated by XBP-1 or a signal transduction pathway involving XBP-1 are identified by contacting cells deficient in XBP-1 ex vivo with one or more test compounds, and determining the effect of the test compound on a read-out. In one embodiment, XBP-1 deficient cells contacted with a test compound ex vivo can be readministered to a subject.

For practicing the screening method ex vivo, cells deficient, e.g., in XBP-1, IRE-1, or ATF6α can be isolated from a non-human XBP-1, IRE-1, or ATF6α deficient animal or embryo by standard methods and incubated (i.e., cultured) in vitro with a test compound. Cells (e.g., B cells) can be isolated from e.g., XBP-1, IRE-1, or ATF6α deficient animals by standard techniques.

In another embodiment, cells deficient in more than one member of a signal transduction pathway involving XBP-1 can be used in the subject assays.

Following contact of the deficient cells with a test compound (either ex vivo or in vivo), the effect of the test compound on the biological response regulated by XBP-1 or a molecule in a signal transduction pathway involving XBP-1 can be determined by any one of a variety of suitable methods, such as those set forth herein, e.g., including light microscopic analysis of the cells, histochemical analysis of the cells, production of proteins, induction of certain genes, e.g., chaperone genes or IL-6.

E. Test Compounds

A variety of test compounds can be evaluated using the screening assays described herein. The term “test compound” includes any reagent or test agent which is employed in the assays of the invention and assayed for its ability to influence the expression and/or activity of XBP-1 or a molecule in a signal transduction pathway involving XBP-1. More than one compound, e.g., a plurality of compounds, can be tested at the same time for their ability to modulate the expression and/or activity of, e.g., XBP-1 in a screening assay. The term “screening assay” preferably refers to assays which test the ability of a plurality of compounds to influence the readout of choice rather than to tests which test the ability of one compound to influence a readout. Preferably, the subject assays identify compounds not previously known to have the effect that is being screened for. In one embodiment, high throughput screening can be used to assay for the activity of a compound.

In certain embodiments, the compounds to be tested can be derived from libraries (i.e., are members of a library of compounds). While the use of libraries of peptides is well established in the art, new techniques have been developed which have allowed the production of mixtures of other compounds, such as benzodiazepines (Bunin et al. (1992). J. Am. Chem. Soc. 114:10987; DeWitt et al. (1993). Proc. NatL. Acad. Sci. USA 90:6909) peptoids (Zuckermann. (1994). J. Med. Chem. 37:2678) oligocarbamates (Cho et al. (1993). Science. 261:1303-), and hydantoins (DeWitt et al. supra). An approach for the synthesis of molecular libraries of small organic molecules with a diversity of 104-105 as been described (Carell et al. (1994). Angew. Chem. Int. Ed. Engl. 33:2059-; Carell et al. (1994) Angew. Chem. Int. Ed. Engl. 33:2061-).

The compounds of the present invention can be obtained using any of the numerous approaches in combinatorial library methods known in the art, including: biological libraries; spatially addressable parallel solid phase or solution phase libraries, synthetic library methods requiring deconvolution, the ‘one-bead one-compound’ library method, and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145). Other exemplary methods for the synthesis of molecular libraries can be found in the art, for example in: Erb et al. (1994). Proc. Natl. Acad. Sci. USA 91:11422-; Horwell et al. (1 996) Immunopharmacology 33:68-; and in Gallop et al. (1 994); J. Med. Chem. 37:1233-.

Libraries of compounds can be presented in solution (e.g, Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad Sci. 87:6378-6382); (Felici (1991) J. Mol. Biol. 222:301-310); In still another embodiment, the combinatorial polypeptides are produced from a cDNA library.

Exemplary compounds which can be screened for activity include, but are not limited to, peptides, nucleic acids, carbohydrates, small organic molecules, and natural product extract libraries.

Candidate/test compounds include, for example, 1) peptides such as soluble peptides, including Ig-tailed fusion peptides and members of random peptide libraries (see, e.g., Lam, K. S. et al. (1991) Nature 354:82-84; Houghten, R. et al. (1991) Nature 354:84-86) and combinatorial chemistry-derived molecular libraries made of D- and/or L-configuration amino acids; 2) phosphopeptides (e.g., members of random and partially degenerate, directed phosphopeptide libraries, see, e.g., Songyang, Z. et al. (1993) Cell 72:767-778); 3) antibodies (e.g., polyclonal, monoclonal, humanized, anti-idiotypic, chimeric, and single chain antibodies as well as Fab, F(ab′)₂, Fab expression library fragments, and epitope-binding fragments of antibodies); 4) small organic and inorganic molecules (e.g., molecules obtained from combinatorial and natural product libraries); 5) enzymes (e.g., endoribonucleases, hydrolases. nucleases, proteases, synthatases, isomerases, polymerases, kinases, phosphatases, oxido-reductases and ATPases), and 6) mutant forms of XBP-1 (or e.g., IRE-1 or ATF6α molecules, e.g., dominant negative mutant forms of the molecules.

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad Sci. U.S.A. 90:6909; Erb et al. (1994) Proc. Natl. Acad. Sci. USA 91:11422; Zuckermann et al. (1994) J. Med Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds can be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.).

Compounds identified in the subject screening assays can be used in methods of modulating one or more of the biological responses regulated by XBP-1. It will be understood that it may be desirable to formulate such compound(s) as pharmaceutical compositions (described supra) prior to contacting them with cells.

Once a test compound is identified that directly or indirectly modulates, e.g., XBP-1 expression or activity, by one of the variety of methods described hereinbefore, the selected test compound (or “compound of interest”) can then be further evaluated for its effect on cells, for example by contacting the compound of interest with cells either in vivo (e.g., by administering the compound of interest to a subject) or ex vivo (e.g., by isolating cells from the subject and contacting the isolated cells with the compound of interest or, alternatively, by contacting the compound of interest with a cell line) and determining the effect of the compound of interest on the cells, as compared to an appropriate control (such as untreated cells or cells treated with a control compound, or carrier, that does not modulate the biological response).

The instant invention also pertains to compounds identified in the subject screening assays.

III. Pharmaceutical Compositions

A pharmaceutical composition of the invention is formulated to be compatible with its intended route of administration. For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid; buffers such as acetates, citrates or phosphates and compounds for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition will preferably be sterile and should be fluid to the extent that easy syringability exists. It will preferably be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal compounds, for example, parabens, chlorobutanol, phenol, ascorbic acid, thimerosal, and the like. In many cases, it will be preferable to include isotonic compounds, for example, sugars, polyalcohols such as manitol, sorbitol, sodium chloride in the composition. Prolonged absorption of the injectable compositions can be brought about by including in the composition an compound which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding compounds, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating compound such as alginic acid, Primogel, or corn starch; a lubricant such as magnesium stearate or Sterotes; a glidant such as colloidal silicon dioxide; a sweetening compound such as sucrose or saccharin; or a flavoring compound such as peppermint, methyl salicylate, or orange flavoring.

In one embodiment, the test compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from, e.g., Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

IV. Methods for Modulating Biological Responses Regulated by XBP-1

The invention also provides for the modulation of various XBP-1 biological activities (e.g., by directly or indirectly modulating XBP-1 or a molecule in a signal transduction pathway involving XBP-1) in cells, e.g., either in vitro or in vivo. In particular, the invention features a method for modulating the UPR in a cell, modulation of cellular differentiation, modulation of IL-6 production, modulation of immunoglobulin production, modulation of the proteasome pathway, modulation of protein folding, secretion, expression and/or transport, modulation of terminal B cell differentiation, and modulation of apoptosis. Accordingly, the invention features methods for modulating one or more biological responses regulated by XBP-1 by contacting the cells with a modulator of XBP-1 expression, processing, post-translational modification, and/or activity such that the biological response is modulated. In another embodiment, a biological response regulated by XBP-1 can be modulated by modulating the expression, processing, post-translational modification, and/or activity of a non-XBP-1 molecule that acts upstream or downstream of XBP-1 in a signal transduction pathway involving XBP-1 (e.g., ATF6α, PERK, or IRE-1). The claimed methods of modulation are not meant to include naturally occurring events. For example, the term “agent” or “modulator” is not meant to embrace endogenous mediators produced by the cells of a subject.

The subject methods employ agents that modulate XBP-1 expression, processing, post-translational modification, or activity (or the expression, processing, post-translational modification, or activity of another molecule in an XBP-1 signaling pathway (e.g., IRE-1)) such that an XBP-1 biological activity is modulated. The subject methods are useful in both clinical and non-clinical settings.

In one embodiment, the instant methods can be performed in vitro. For example, the production of a commercially valuable protein, e.g., a recombinantly expressed protein, can be increased by stimulating the expression, processing, post-translational modification, and/or activity of spliced XBP-1 or by inhibiting the expression, processing, post-translational modification, and/or activity of a negative regulator of spliced XBP-1. In a preferred embodiment, the production of immunoglobulin can be increased in a cell either in vitro or in vivo. In another embodiment, XBP -1 expression, processing, post-translational modification, and/or activity can be modulated in a cell in vitro and then the treated cells can be administered to a subject.

In one embodiment, the methods and compositions of the invention can be used to modulate XBP-1 expression, processing, post-translational modification, and/or activity (or the expression, processing, post-translational modification, and/or activity of a molecule in a signal transduction pathway involving XBP-1) in a cell. In one embodiment, the cell is a mammalian cell. In another embodiment, the cell is a human cell. Such modulation can occur in vitro or in vivo. The subject invention can also be used to treat various conditions or disorders that would benefit from modulation of one or more XBP-1 biological activity. In one embodiment, cells in which, e.g., XBP-1, is modulated in vitro can be introduced or reintroduced into a subject. In one embodiment, the invention also allows for modulation of XBP-1 in vivo, by administering to the subject a therapeutically effective amount of a modulator of XBP-1 such that a biological effect of XBP-1 in a subject is modulated. For example, XBP-1 can be modulated to treat a malignancy, an autoimmune disorder, or an immunodeficiency.

In one embodiment, an agent that downmodulates e.g., the expression, processing, post-translational modification, and/or activity of spliced XBP-1 or a molecule in a signal transduction pathway involving XBP-1 is contacted with a cell to downmodulate the UPR. In one embodiment, the cell is a secretory cell.

In one embodiment, the cell is a malignant cell.

In another embodiment, the agent used to modulate expression, processing, post-translational modification, and/or activity of XBP-1 or a molecule in a signal transduction pathway involving XBP-1 is not monomeric boronic acid such as [(1R)-3-methyl-1-[[(2S)-1-oxo-3-phenyl-2-[(pyrazinylcarbonyl)amino]propyl]amino]butyl]boronic acid, Velcade™).

In one embodiment, the agent is not a compound of the formula (I):

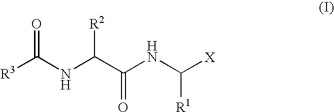

wherein:

X is B(OR⁴)(OR⁵), CHO, or C(═O)NR⁶′R⁶;

R¹ and R² are each independently alkyl (methyl, ethyl, isopropyl, etc.), aryl (e.g., phenyl, hydroxy phenyl), aralkyl (e.g., benzyl), or the side chain of a hydrophobic amino acid (e.g., leucine, valine, isoleucine, phenylalanine, or alanine);

R³ is heterocyclic(e.g., a 5 or 6 membered ring with 1, 2, or 3 heteroatoms), carbocyclic (e.g., indanone), aralkyl, or [AA]₁₋₃-Z, wherein AA is a D or L amino acid and Z is heterocyclic, aryl, benzoylcarbonyl, benzoylglycine, t-butoxycarbonyl, 9H-fluoren-9-ylmethyloxycarbonyl (Fmoc), lower alkoyl, or acetyl;

R⁴ and R⁵ are each independently lower alkyl (e.g., methyl, ethyl, etc.), hydrogen, aryl, or araalkyl;

R⁶ and R⁶′ are each independently an amino acid, hydrogen, optionally substituted lower alkyl, optionaily substituted aryl, or optionally substituted aralkyl and pharmaceutically acceptable salts thereof.

In one embodiment, the agent used to modulate expression. processing, post-translational modification, and/or activity of XBP-1 or a molecule in a signal transduction pathway involving XBP-1 is not a proteasome inhibitor of the dipeptidyl boronate class. The term “dipeptidyl boronate” includes compounds of formula I wherein X is B(OR⁴)(OR⁵). It includes compounds which comprise at least two peptidyl bonds (C(═O)—NR—) and a boronic acid moiety or a derivative thereof (e.g., boronic esters).

In a further embodiment, the dipeptidyl boronate includes compounds wherein X is B(OH)₂. In another further embodiment, R¹ is alkyl (e.g., isopropyl) and R² is aralkyl (e.g., benzyl). In another further embodiment, R³ is heterocyclic (e.g., pyrazinyl).

In another embodiment, tne agent does not include tetrapeptidyl 30 aldehydes (or other compounds) described in U.S. Pat. No. 5,580,854 or U.S. Patent Application No. 2002/0111314, each of which are incorporated herein by reference. In another embodiment, the agent does not include the di- or tri-peptidyl aldehyde derivatives (or other compounds) described in U.S. Pat. No. 5,693,617, incorporated herein by reference. In another embodiment, the agent is not a boronic acid, ester or other compound described in U.S. Pat. Nos. 6,548,668, 6,083,903, or WO 03/033506, each of which are incorporated herein by reference. In another embodiment, the agents do not include the α-ketoamide compounds (and other compounds) described in U.S. Pat. No. 6,075,150 or WO/99/37666, incorporated herein by reference. In another embodiment, the agent do not include the dipeptidyl indanones (or other compounds) described in U.S. Pat. No. 6,117,887, incorporated herein by reference.

In another embodiment, the agent is not a clasto-lactacystin-β-lactone or other lactacystin analog described in U.S. Pat. Nos. 6,133,308, 6,214,862, 6,458,825, or WO 99/22729, each of which are incorporated herein by reference.

In another embodiment, the agent is not a benzylmalonic acid derivative or other compound described in WO 03/033507, incorporated herein by reference. In another embodiment, the agent is not a carboxylic acid derivative described in WO 00/43000.

In another embodiment, the agent is not a tea-derived polyphenol as described in US 2002/0151582, incorporated herein by reference. In another embodiment, the compound is not a 2-amino-3-hydroxy-4-tert-leucyl-amino-5-phenyl-pentanoic acid derivative described in WO 01/89282, incorporated herein by reference.

In another embodiment, an agent used to modulate expression, processing, post-translational modification, and/or activity of XBP-1 or a molecule in a signal transduction pathway involving XBP-1 is not a proteasome inhibitor. For example, in one embodiment, the agent does not directly (e.g., either reversibly or irreversibly) inhibit the 26S proteasome. Such non-proteasome inhibitor agents do not exert their primary effects on cells by inhibiting the degradation of cellular proteins, e.g., NF-κB inhibitors, such as IκBα. In one embodiment of the invention, a modulatory agent of the invention modulates XBP-1 (or a molecule in a signal transduction pathway involving XBP-1) without substantially modulating the NF-KB pathway, e.g., as measured by measuring the degradation of IκBα.

In one embodiment, a modulatory agent of the invention does not interfere with the initial steps of IRE1α activation, i.e., does not result in impaired oligomerization and autophosphorylation. In another embodiment, a modulatory agent of the invention directly affects the expression, post-translational modification, and/or activity of XBP-1 protein. In one embodiment, the expression of XBP-1 is modulated.

In another embodiment, the post-translational modification of XBP-1 is modulated. In another embodiment, the activity of XBP-1 is modulated.

In one embodiment, an agent of the invention preferentially kills cells which are particularly dependent on the unfolded protein response to survive, e.g., secretory cells.

The term “subject” is intended to include living organisms but preferred subjects are mammals. Examples of subjects include mammals such as, e.g., humans,/monkeys, dogs, cats, mice, rats cows, horses, goats, and sheep.

Identification of compounds that modulate the biological effects of XBP-1 by directly or indirectly modulating XBP-1 activity allows for selective manipulation of these biological effects in a variety of clinical situations using the modulatory methods of the invention. For example, the stimulatory methods of the invention (i.e., methods that use a stimulatory agent) can result in increased expression, processing, post-translational modification, and/or activity of spliced XBP-1, which stimulates, e.g., IL-6 production, plasma cell differentiation, protein folding and transport, and immunoglobulin production. In another embodiment, the stimulatory methods of the invention can be used to increase the expression, processing, post-translational modification, and/or activity of a negative regulator of XBP-1 (e.g., unspliced XBP-1 or a dominant negative form of XBP-1) to inhibit e.g., IL-6 production, plasma cell differentiation, protein folding and transport, and immunoglobulin production.

In one embodiment, of the invention, modulation of XBP-1 expression, processing, post-translational modification, and/or activity results in at least about a 2-fold difference in IL-6 production in a cell. In another embodiment, modulation of XBP-1 results in at least about a 5-fold difference in IL-6 production in a cell. In yet another embodiment, modulation of XBP-1 results in at least about a 10-fold difference in IL-6 production by a cell.

In contrast, the inhibitory methods of the invention (i.e., methods that use an inhibitory agent) can inhibit the activity of spliced XBP-1 and inhibit IL-6 production, plasma cell differentiation, protein folding and transport, and immunoglobulin production, as demonstrated in the Examples.

In another embodiment, the inhibitory methods of the invention inhibit the activity of a negative regulator of XBP-1, e.g., unspliced XBP-1 or a dominant negative form of XBP-1. The XBP-1 unspliced protein is an example of a ubiquitinated and hence extremely unstable protein. XBP-1 spliced protein is not ubiquitinated, and has a much longer half life than unspliced XBP-1 protein. Proteasome inhibitors, for example, block ubiquitination, and hence stabilize XBP-1 unspliced but not spliced protein. Thus, the ratio of unspliced to spliced XBP-1 protein increases upon treatment with proteasome inhibitors. Since unspliced XBP-1 protein actually inhibits the function of the spliced protein, treatment with proteasome inhibitors blocks the activity of spliced XBP-1.

Modulation of XBP-1 activity, therefore, provides a means to regulate disorders arising from aberrant XBP-1 activity in various disease states. Thus, to treat a disorder wherein inhibition of a biological effect of spliced XBP-1 is desirable, such as a disorder that would benefit from reduced cellular differentiation, downmodulation of the UPR, downmodulation of IL-6 production, downmodulation of immunoglobulin production, downmodulation of the proteasome pathway, downmodulation of protein folding and transport, downmodulation of terminal B cell differentiation, or modulation of apoptosis (e.g., an autoimmune disorder or a malignancy (e.g., multiple myeloma)) is beneficial, an inhibitory method of the invention is selected such that spliced XBP-1 activity and/or expression is inhibited or a stimulatory method is selected which selectively stimulates the expression and/or activity of a negative regulator of XBP-1. Examples of disorders in which such inhibitory methods can be useful include multiple myeloma and autoimmune diseases, in particular those characterized by the production of pathogenic autoantibodies. The activity of spliced XBP-1 can also be decreased, for example to promote immunotolerization, e.g, to allergens.

Alternatively, to treat a disorder wherein stimulation of a biological effect of spliced XBP-1 is desirable, such as a disorder that would benefit from increased cellular differentiation, upmodulation of the UPR, upmodulation of IL-6 production, upmodulation of immunoglobulin production, upmodulation of the proteasome pathway, upmedulation of protein folding and transport, upmodulation of terminal B cell differentiation, or modulation of apoptosis (e.g., a malignancy that would benefit from an anti-tumor immune response or an acquired immunodeficiency disorder), a stimulatory method of the invention is selected such that spliced XBP-1 activity and/or expression is upregulated or an inhibitory method is selected such that the expression and/or activity of a negative regulator of XBP-1 is inhibited. In addition, as set forth in more detail below, increasing spliced XBP-1 activity is useful, e.g., in improving humoral responses to pathogens (e.g., viruses, microbes, or parasites) in a subject and for improving the efficacy of vaccination in a subject.

In one embodiment, the modulatory methods of the invention are practiced on a subject in a patient population that would benefit from modulation of a signal transduction pathway involving XBP-1. For example, in one embodiment, the modulatory methods of the invention are practiced on a subject that would benefit from modulation of the UPR. In one embodiment, the modulatory methods of the invention are practiced on a subject identified as one that would benefit from modulation of a signal transduction pathway involving XBP-1 using a diagnostic method of the invention. For example, in one embodiment, a patient is identified as one that would benefit from modulation of a signal transduction pathway involving XBP-1 or modulation of an XBP-1 activity. This can be done, e.g., using one of the diagnostic methods described herein. For example, a biological specimen can be obtained from the patient and assayed for, e.g., expression or activity of XBP-1 or a molecule in a signal transduction pathway involving XBP-1.

In another embodiment, a biological sample from a subject can be examined for the presence of mutations in a gene encoding XBP-1 (or a molecule in a signal transduction pathway encoding XBP-1) or in the promoter region for XBP-1 (or a gene in a signal transduction pathway encoding XBP-1).

In another embodiment, the level of expression of genes whose expression is regulated by XBP-1 (e.g., ERdj4, p58^{IPK}, EDEM, PDI-P5, RAMP4, BiP, XBP-1, or ATF6α) can be measured using standard techniques.

In another embodiment, a subject is identified as one that would benefit from modulation of a signal transduction pathway involving XBP-1 or modulation of an XBP-1 activity by examining certain cells of the subject to determine whether they are secretory cells. For example, in one embodiment, a malignancy in a subject is examined (e.g., using standard histological techniques) to determine whether the malignancy originated in secretory cells.

In one embodiment, a subject is treated with an agent that modulates a signal transduction pathway involving XBP-1 in an amount sufficient to modulate the expression, processing, post-translational modification, and/or activity of XBP-1 protein or a protein in a signal transduction pathway involving XBP-1. For example, in one embodiment, a subject is treated with an agent in an amount sufficient to modulate an activity of XBP-1, e.g., the unfolded protein response, in a cell of the subject.

Application of the modulatory methods of the invention to the treatment of a disorder can result in curing the disorder, a decrease in the type or number of symptoms associated with the disorder, either in the long term or short term (i.e., amelioration of the condition) or simply a transient beneficial effect to the subject.

Application of the immunomodulatory methods of the invention is described in further detail below.

A. Inhibitory Compounds

The methods of the invention using inhibitory compounds which inhibit the expression, processing, post-translational modification, or activity of spliced XBP-1 or a molecule in a signal transduction pathway involving XBP-1 can be used in the treatment of disorders in which spliced XBP-1 activity is undesirably enhanced, stimulated, upregulated or the like. For example, multiple myelomas and certain autoimmune diseases are associated with increased immunoglobulin production by plasma cells. Accordingly, preferred disorders for treatment using an inhibitory compound of the invention include, e.g., multiple myeloma and autoimmune disorders characterized by increased immunoglobulin production.

In another embodiment, inhibitory compounds can be used to inhibit the expression, processing, post-translational modification, or activity of a negative regulator of XBP-1, e.g., unspliced XBP-1. Such compounds can be used in the treatment of disorders in which unspliced XBP-1 is undesirably elevated or when spliced XBP-1 expression and/or activity is undesirably reduced.

In one embodiment of the invention, an inhibitory compound can be used to inhibit (e.g., specifically inhibit) the expression, processing, post-translational modification, or activity of spliced XBP-1. In another embodiment, an inhibitory compound can be used to inhibit (e.g., specifically inhibit) the expression, processing, post-translational modification, or activity of unspliced XBP-1.

Inhibitory compounds of the invention can be, for example, intracellular binding molecules that act to specifically inhibit the expression, processing, post-translational modification, or activity e.g., of XBP-1 or a molecule in a signal transduction pathway involving XBP-1(e.g., IRE-1 or ATF6α). As used herein, the term “intracellular binding molecule” is intended to include molecules that act intracellularly to inhibit the processing expression or activity of a protein by binding to the protein or to a nucleic acid (e.g., an mRNA molecule) that encodes the protein. Examples of intracellular binding molecules, described in further detail below, include arttisense nucleic acids, intracellular antibodies, peptidic compounds that inhibit the interaction of XBP-1 or a molecule in a signal transduction pathway involving XBP-1 with a target molecule and chemical agents that specifically inhibit XBP-1 activity or the activity of a molecule in a signal transduction pathway involving XBP-1.

i. Antisense or siRNA Nucleic Acid Molecules

In one embodiment, an inhibitory compound of the invention is an antisense nucleic acid molecule that is complementary to a gene encoding XBP-1 or a molecule in a signal transduction pathway involving XBP-1, e.g., a molecule with which XBP-1 interacts), or to a portion of said gene, or a recombinant expression vector encoding said antisense nucleic acid molecule. The use of antisense nucleic acids to downregulate the expression of a particular protein in a cell is well known in the art (see e.g., Weintraub, H. et al., Antisense RNA as a molecular tool for genetic analysis, Reviews—Trends in Genetics, Vol. 1(1) 1986; Askari, F. K. and McDonnell, W. M. (1996) N. Eng. J. Med. 334:316-318; Bennett, M. R. and Schwartz, S. M. (1995) Circulation 92:1981-1993; Mercola, D. and Cohen, J. S. (1995) Cancer Gene Ther. 2:47-59; Rossi, J. J. (1995) Br. Med. Bull. 51 :217-225; Wagner, R. W. (1994) Nature 372:333-335). An antisense nucleic acid molecule comprises a nucleotide sequence that is complementary to the coding strand of another nucleic acid molecule (e.g., an mRNA sequence) and accordingly is capable of hydrogen bonding to the coding strand of the other nucleic acid molecule. Antisense sequences complementary to a sequence of an mRNA can be complementary to a sequence found in the coding region of the mRNA, the 5′ or 3′ untranslated region of the mRNA or a region bridging the coding region and an untranslated region (e.g., at the junction of the 5′ untranslated region and the coding region). Furthermore, an antisense nucleic acid can be complementary in sequence to a regulatory region of the gene encoding the mRNA, for instance a transcription initiation sequence or regulatory element. Preferably, an antisense nucleic acid is designed so as to be complementary to a region preceding or spanning the initiation codon on the coding strand or in the 3′ untranslated region of an mRNA.

Given the known nucleotide sequence for the coding strand of the XBP-1 gene (or e.g., the IRE-1 or ATF6α gene) and thus the known sequence of the XBP-1. IRE-1, or ATF6α mRNA, antisense nucleic acids of the invention can be designed according to the rules of Watson and Crick base pairing. The antisense nucleic acid molecule can be complementary to the entire coding region of an mRNA, but more preferably is antisense to only a portion of the coding or noncoding region of an mRNA. For example, the antisense oligonucleotide can be complementary to the region surrounding the translation start site of an XBP-1 (or e.g., the IRE-1 or ATF6α) mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. To inhibit expression in cells, one or more antisense oligonucleotides can be used.

Alternatively, an antisense nucleic acid can be produced biologically using an expression vector into which all or a portion of a cDNA has been subcloned in an antisense orientation (i.e., nucleic acid transcribed from the inserted nucleic acid will be of an antisense orientation to a target nucleic acid of interest). Regulatory sequences operatively linked to a nucleic acid cloned in the antisense orientation can be chosen which direct the expression of the antisense RNA molecule in a cell of interest, for instance promoters and/or enhancers or other regulatory sequences can be chosen which direct constitutive, tissue specific or inducible expression of antisense RNA. The antisense expression vector is prepared according to standard recombinant DNA methods for constructing recombinant expression vectors, except that the cDNA (or portion thereof) is cloned into the vector in the antisense orientation. The antisense expression vector can be in the form of, for example, a recombinant plasmid, phagemid or attenuated virus. The antisense expression vector can be introduced into cells using a standard transfection technique.

The antisense nucleic acid molecules of the invention are typically administered to a subject or generated in situ such that they hybridize with or bind to cellular mRNA and/or genomic DNA encoding a protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of an antisense nucleic acid molecule of the invention includes direct injection at a tissue site. Alternatively, an antisense nucleic acid molecule can be modified to target selected cells and then administered systemically. For example, for systemic administration, an antisense molecule can be modified such that it specifically binds to a receptor or an antigen expressed on a selected cell surface, e.g., by linking the antisense nucleic acid molecule to a peptide or an antibody which binds to a cell surface receptor or antigen. The antisense nucleic acid molecule can also be delivered to cells using the vectors described herein. To achieve sufficient intracellular concentrations of antisense molecules, vector constructs in which the antisense nucleic acid molecule is placed under the control of a strong pol II or pol III promoter are preferred.

In yet another embodiment, an antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid molecule of the invention is a ribozyme. Ribozymes are catalytic RNA molecules with ribonuclease activity which are capable of cleaving a single-stranded nucleic acid, such as an mRNA, to wnich they have a complementary region. Thus, ribozymes (e.g., hamrnmerhead ribozymes (described in Haselhoff and Gerlach (1988) Nature 334:585-591)) can be used to catalytically cleave mRNA transcripts to thereby inhibit translation mRNAs. A ribozyme having specificity e.g., for an XBP-1, IRE-1, or ATF6α-encoding nucleic acid can be designed based upon the nucleotide sequence of the cDNA. For example, a derivative of a Tetrahymena L-19 IVS RNA can be constructed in which the nucleotide sequence of the active site is complementary to the nucleotide sequence to be cleaved in, e.g., an XBP-1, IRE-1, or ATF6α-encoding mRNA. See, e.g., Cech et al. U.S. Pat. No. 4,987,071 and Cech et al. U.S. Pat. No. 5,116,742. Alternatively, XBP-1 (or, e.g., IRE-1, ATF6α) mRNA can be used to select a catalytic RNA having a specific ribonuclease activity from a pool of RNA molecules. See, e.g., Bartel, D. and Szostak, J. W. (1993) Science 261:1411-1418.

Alternatively, gene expression can be inhibited by targeting nucleotide sequences complementary to the regulatory region of a gene (e.g., an XBP-1, IRE-1, or ATF6α promoter and/or enhancer) to form triple helical structures that prevent transcription of a gene in target cells. See generally, Helene, C. (1991) Anticancer Drug Des. 6(6):569-84; Helene, C. et al. (1992) Ann. N.Y. Acad. Sci. 660:27-36; and Maher, L. J. (1992) Bioassays 14(12):807-15.

In another embodiment, a compound that promotes RNAi can be used to inhibit expression of XBP-1 or a molecule in a signal transduction pathway involving XBP-1. RNA interference (RNAi is a post-transcriptional, targeted gene-silencing technique that uses double-stranded RNA (dsRNA) to degrade messenger RNA (mRNA) containing the same sequence as the dsRNA (Sharp, P. A. and Zamore, P. D. 287, 2431-2432 (2000); Zamore, P. D., et al. Cell 101, 25-33 (2000). Tuschl, T. et al. Genes Dev. 13, 3191-3197 (1999); Cottrell TR, and Doering TL. 2003. Trends Microbiol. 11:37-43; Bushman F.2003. Mol Therapy. 7:9-10; McManus MT and Sharp PA. 2002. Nat Rev Genet. 3:737-47). The process occurs when an endogenous ribonuclease cleaves the longer dsRNA into shorter, e.g., 21- or 22-nucleotide-long RNAs, termed small interfering RNAs or siRNAs. The smaller RNA segments then mediate the degradation of the target mRNA. Kits for synthesis of RNAi are commercially available from, e.g. New England Biolabsor Ambion. In one embodiment one or more of the chemistries described above for use in antisense RNA can be employed in molecules that mediate RNAi. A working example of XBP-1 specific RNAi is described in the appended Examples, in which an XBP-1-specific RNAi vector was constructed by inserting two complementary oligonucleotides for 5′-GGGATTCATGAATGGCCCTTA-3′ (SEQ ID NO: 11) into the pBS/U6 vector.

ii. Intracellular Antibodies

Another type of inhibitory compound that can be used to inhibit the expression and/or activity of XBP-1 or a molecule in a signal transduction pathway involving XBP-1 is an intracellular antibody specific for, e.g., XBP-1, IRE-1, or ATF6α or another molecule in the pathway as discussed herein. In one embodiment, an antibody binds to both spliced and unspliced XBP-1. In another embodiment, an antibody is specific for spliced XBP-1, i.e., recognizes an epitope present in ORF2. The use of intracellular antibodies to inhibit protein function in a cell is known in the art (see e.g., Carlson, J. R. (1988) Mol. Cell. Biol. 8:2638-2646; Biocca, S. et al. (1990) EMBO J. 9:101-108; Werge, T. M. et al. (1990) FEBS Letters 274:193-198; Carlson, J. R. (1993) Proc. Natl. Acad. Sci. USA 90:7427-7428; Marasco, W. A. et al. (1993) Proc. Natl. Acad. Sci. USA 90:7889-7893; Biocca, S. et al. (1994) Bio/Technology 12:396-399; Chen, S-Y. et al. (1994) Human Gene Therapy 5:595-601; Duan, L et al. (1994) Proc. Natl. Acad. Sci. USA 91:5075-5079; Chen, S-Y. et al. (1994) Proc. Natl. Acad. Sci. USA 91:5932-5936; Beerli, R. R. et al. (1994) J. Biol. Chem. 269:23931-23936; Beerli, R. R. et al. (1994) Biochem. Biophys. Res. Commun. 204:666-672; Mhashilkar, A. M. et al. (1995) EMBO J. 14:1542-1551; Richardson, J. H. et al. (1995) Proc. Natl. Acad Sci. USA 92:3137-3141; PCT Publication No. WO 94/02610 by Marasco et al.; and PCT Publication No. WO 95/03832 by Duan et al.).

To inhibit protein activity using an intracellular antibody, a recombinant expression vector is prepared which encodes the antibody chains in a form such that, upon introduction of the vector into a cell, the antibody chains are expressed as a functional antibody in an intracellular compartment of the cell. For inhibition of transcription factor activity according to the inhibitory methods of the invention, preferably an intracellular antibody that specifically binds the protein is expressed within the nucleus of the cell. Nuclear expression of an intracellular antibody can be accomplished by removing from the antibody light and heavy chain genes those nucleotide sequences that encode the N-terminal hydrophobic leader sequences and adding nucleotide sequences encoding a nuclear localization signal at either the N- or C-terminus of the light and heavy chain genes (see e.g., Biocca, S. et al. (1990) EMBO J. 9:101-108; Mhashilkar, A. M. et al. (1995) EMBO J. 14:1542-1551). A preferred nuclear localization signal to be used for nuclear targeting of the intracellular antibody chains is the nuclear localization signal of SV40 Large T antigen (see Biocca, S. et al. (1990) EMBO J. 9:101-108; Mhashilkar, A. M. et al. (1995) EMBO J. 14:1542-1551).

To prepare an intracellular antibody expression vector, antibody light and heavy chain cDNAs encoding antibody chains specific for the target protein of interest, e.g., XBP-1, IRE-1, or ATF6α protein, is isolated, typically from a hybridoma that secretes a monoclonal antibody specific for the protein. Antibodies can be prepared by immunizing a suitable subject, (e.g., rabbit, goat, mouse or other mammal), e.g., with an XBP-1, IRE-1, or ATF6α protein immunogen. An appropriate immunogenic preparation can contain, for example, recombinantly expressed protein or a chemically synthesized peptide. The preparation can further include an adjuvant, such as Freund's complete or incomplete adjuvant, or similar immunostimulatory compound. Antibody-producing cells can be obtained from the subject and used to prepare monoclonal antibodies by standard techniques, such as the hybridoma technique originally described by Kohler and Milstein (1975, Nature 256:495-497) (see also, Brown et al. (1981) J. Immunol 127:539-46; Brown et al. (1980) J Biol Chem 255:4980-83; Yeh et al. (1976) PNAS 76:2927-31; and Yeh et al. (1982) Int. J. Cancer 29:269-75). The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lemer (1981) Yale J. Biol. Med, 54:387-402; M. L. Gefter et al. (1977) Somatic Cell Genet., 3:231-36). Briefly, an immortal cell line (typically a myeloma) is fused to lymphocytes (typically splenocytes) from a mammal immunized with a protein immunogen as described above, and the culture supernatants of the resulting hybridoma cells are screened to identify a hybridoma producing a monoclonal antibody that binds specifically, e.g., to the XBP-1, IRE-1, or ATF6α protein. Any of the many well known protocols used for fusing lymphocytes and immortalized cell lines can be applied for the purpose of generating a monoclonal antibody (see, e.g., G. Galfre et al. (1977) Nature 266:550-52; Gefter et al. Somatic Cell Genet., cited supra; Lemer, Yale J. Biol. Med., cited supra; Kenneth, Monoclonal Antibodies, cited supra). Moreover, the ordinary skilled artisan will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from the American Type Culture Collection (ATCC), Rockville, Md. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody that specifically binds the protein are identified by screening the hybridoma culture supernatants for such antibodies, e.g., using a standard ELISA assay.

Alternative to preparing monoclonal antibody-secreting hybridomas, a monoclonal antibody that binds to a protein can be identified and isolated by screening a recombinant combinatorial immunoglobulin library (e.g., an antibody phage display library) with the protein, or a peptide thereof, to thereby isolate immunoglobulin library members that bind specifically to the protein. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and compounds particularly amenable for use in generating and screening antibody display library can be found in, for example, Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Fuchs et al. (1991) Bio/Technology 9:1370-1372; Hay et al. (1992) Hum Antibod Hybridomas 3:81-85; Huse et al. (1989) Science 246:1275-1281; Griffiths et al. (1993) EMBO J 12:725-734; Hawkins et al. (1992) J Mol Biol 226:889-896; Clarkson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; Barbas et al. (1991) PNAS 88:7978-7982; and McCafferty et al. Nature (1990) 348:552-554.

In another embodiment, ribosomal display can be used to replace bacteriophage as the display platform (see, e.g., Hanes et al. 2000. Nat. Biotechnol. 18:1287; Wilson et al. 2001. Proc. Natl. Acad Sci. USA 98:3750; or Irving et al. 2001 J. Immunol. Methods 248:31. In yet another embodiment, cell surface libraries can be screened for antibodies (Boder et al. 2000. Proc. Natl. Acad. Sci. USA 97:10701; Daugherty et al. 2000 J. Immunol. Methods 243:211. Such procedures provide alternatives to traditional hybridoma techniques for the isolation and subsequent cloning of monoclonal antibodies.

Yet other embodiments of the present invention comprise the generation of substantially human antibodies in transgenic animals (e.g., mice) that are incapable of endogenous immunoglobulin production (see e.g., U.S. Pat. Nos. 6,075,181, 5,939,598, 5,591,669 and 5,589,369 each of which is incorporated herein by reference). For example, it has been described that the homozygous deletion of the antibody heavy-chain joining region in chimeric and germ-line mutant mice results in complete inhibition of endogenous antibody production. Transfer of a human immunoglobulin gene array to such germ line mutant mice will result in the production of human antibodies upon antigen challenge. Another preferred means of generating human antibodies using SCID mice is disclosed in U.S. Pat. No. 5,811,524 which is incorporated herein by reference. It will be appreciated that the genetic material associated with these human antibodies can also be isolated and manipulated as described herein.

Yet another highly efficient means for generating recombinant antibodies is disclosed by Newman, Biotechnology, 10: 1455-1460 (1992). Specifically, this technique results in the generation of primatized antibodies that contain monkey variable domains and human constant sequences. This reference is incorporated by reference in its entirety herein. Moreover, this technique is also described in U.S. Pat. Nos. 5,658,570, 5,693,780 and 5,756,096 each of which is incorporated herein by reference.

Once a monoclonal antibody of has been identified (e.g., either a hybridoma-derived monoclonal antibody or a recombinant antibody from a combinatorial library, including monoclonal antibodies that are already known in the art), DNAs encoding the light and heavy chains of the monocional antibody are isolated by standard molecular biology techniques. For hybridoma derived antibodies, light and heavy chain cDNAs can be obtained, for example, by PCR amplification or cDNA library screening. For recombinant antibodies, such as from a phage display library, cDNA encoding the light and heavy chains can be recovered from the display package (e.g., phage) isolated during the library screening process. Nucleotide sequences of antibody light and heavy chain genes from which PCR primers or cDNA library probes can be prepared are known in the art. For example, many such sequences are disclosed in Kabat, E. A., et al. (1991) Sequences of Proteins of Immunological Interest, Fifth Edition, U.S. Department of Health and Human Services, NIH Publication No. 91-3242 and in the “Vbase” human germline sequence database.

Once obtained, the antibody light and heavy chain sequences are cloned into a recombinant expression vector using standard methods. As discussed above, the sequences encoding the hydrophobic leaders of the light and heavy chains are removed and sequences encoding a nuclear localization signal (e.g., from SV40 Large T antigen) are linked in-frame to sequences encoding either the amino- or carboxy terminus of both the light and heavy chains. The expression vector can encode an intracellular antibody in one of several different forms. For example, in one embodiment, the vector encodes full-length antibody light and heavy chains such that a full-length antibody is expressed intracellularly. In another embodiment, the vector encodes a full-length light chain but only the VH/CH1 region of the heavy chain such that a Fab fragment is expressed intraceilularly. In the most preferred embodiment, the vector encodes a single chain antibody (scFv) wherein the variable regions of the light and heavy chains are linked by a flexible peptide linker (e.g., (Gly₄Ser)₃) and expressed as a single chain molecule. To inhibit transcription factor activity in a cell, the expression vector encoding, e.g., the XBP-1, IRE-1, or ATF6α-specific intracellular antibody is introduced into the cell by standard transfection methods as described hereinbefore.

iii. Peptidic Compounds

In another embodiment, an inhibitory compound of the invention is a peptidic compound derived from the XBP-1 amino acid sequence or the amino acid sequence of a molecule in a signal transduction pathway involving XBP-1 (e.g., IRE-1, or ATF6α). For example, in one embodiment, the inhibitory compound comprises a portion of, e.g., XBP-1, IRE-1, or ATF6α (or a mimetic thereof) that mediates interaction of XBP-1, IRE-1, or ATF6α with a target molecule such that contact of XBP-1, IRE-1, or ATF6α with this peptidic compound competitively inhibits the interaction of XBP-1, IRE-1, or ATF6α with the target molecule.

The peptidic compounds of the invention can be made intracellularly in cells by introducing into the cells an expression vector encoding the peptide. Such expression vectors can be made by standard techniques using oligonucleotides that encode the amino acid sequence of the peptidic compound. The peptide can be expressed in intracellularly as a fusion with another protein or peptide (e.g., a GST fusion). Alternative to recombinant synthesis of the peptides in the cells, the peptides can be made by chemical synthesis using standard peptide synthesis techniques. Synthesized peptides can then be introduced into cells by a variety of means known in the art for introducing peptides into cells (e.g., liposome and the like). In addition, dominant negative proteins (e.g., of XBP-1, IRE-1, or ATF6α) can be made which include XBP-1, IRE-1, or ATF6α molecules (e.g., portions or variants thereof) that compete with native (i.e., wild-type) molecules, but which do not have the same biological activity. Such molecules effectively decrease, e.g., XBP-1, IRE-1, or ATF6α activity in a cell. For example, the peptide compound can be lacking part of an XBP-1 transcriptional activation domain, e.g., can consist of the portion of the N-terminal 136 or 188 amino acids of the spliced form of XBP-1.

iv. Other Agents that Act Upstream of XBP-1

In one embodiment, the expression of spliced XBP-1 can be inhibited using an agent that inhibits a signal that increases XBP-1 expression, processing, post-translational modification or activity in a cell. Both IL-4 and IL-6 have been shown to increase transcription of XBP-1 (Wen et al. 1999. Int. Journal of Oncology 15:173). Accordingly, in one embodiment, an agent that inhibits a signal transduced by IL-4 or IL-6 can be used to downmodulate XBP-1 expression and, thereby, decrease the activity of spliced XBP-1 in a cell. For example, in one embodiment, an agent that inhibits a STAT-6 dependent signal can be used to decrease the expression of XBP-1 in a cell. In another embodiment, an agent that interferes with a CD40-mediated signal (e.g., by reducing CD40 expression or CD40-mediated signaling) in a B cell can be used to downmodulate spliced XBP-1 activity.

Other inhibitory agents that can be used to specifically inhibit the activity of an XBP-1 or a molecule in a signal transduction pathway involving XBP-1 are chemical compounds that directly inhibit expression, processing, post-translational modification, and/or activity of, e.g., an XBP-1, IRE-1, or ATF6α target protein activity or inhibit the interaction between, e.g., XBP-1, IRE-1, or ATF6α and target molecules. Such compounds can be identified using screening assays that select for such compounds, as described in detail above as well as using other art recognized techniques.

B. Stimulatory Compounds

The methods of the invention using spliced XBP-1 stimulatory compounds can be used in the treatment of disorders in which spliced XBP activity and/or expression is undesirably reduced, inhibited, downregulated or the like. For example, in the case of malignancies which would benefit from enhanced anti-tumor immune responses (e.g., antibody responses) and certain acquired immune deficiencies. In one embodiment, the stimulatory methods of the invention, a subject is treated with a stimulatory compound that stimulates expression and/or activity of spliced XBP-1 or a molecule in a signal transduction pathway involving XBP-1.

In another embodiment, a stimulatory method of the invention can be used to stimulate the expression and/or activity of a negative regulator of spliced XBP-1 activity.

The methods of the invention using spliced XBP-1 stimulatory compounds can be used in the treatment of disorders in which the UPR or the proteasome pathway protein is inhibited, blocked, downregulated or the like, e.g., when cellular differentiation or production of one or more molecules whose transcription is regulated by IL-6 is desired or when reduced apoptosis is desired. Moreover, the stimulatory methods that stimulate the expression and/or activity of spliced XBP-1 of the invention are of general use in the stimulation of humoral immune responses to pathogens in a subject and in improving antibody responses during vaccination of a subject.

Molecules that stimulate the expression and/or activity of a negative regulator of XBP-1 can be used in the treatment of disorders in which the UPR, the proteasome pathway would benefit from being downregulated, e.g. in the case of an autoimmune disease, a malignancy, or where a decrease in cellular differentiation or production of one or more proteins whose expression is regulated by XBP-1 should be downregulated. In addition, molecules that stimulate the expression and/or activity of a negative regulator of XBP-1 can be used to stimulate apoptosis.

Examples of stimulatory compounds include proteins, expression vectors comprising nucleic acid molecules and chemical agents that stimulate expression and/or activity of the protein of interest.

A preferred stimulatory compound is a nucleic acid molecule encoding unspliced XBP-1 that is capable of being spliced or spliced XBP wherein the nucleic acid molecule is introduced into the subject in a form suitable for expression of the protein in the cells of the subject. For example, an XBP-1 cDNA (full length or partial cDNA sequence) is cloned into a recombinant expression vector and the vector is transfected into cells using standard molecular biology techniques. The XBP-1 cDNA can be obtained, for example, by amplification using the polymerase chain reaction (PCR) or by screening an appropriate cDNA library. The nucleotide sequences of XBP-1 cDNA are known in the art and can be used for the design of PCR primers that allow for amplification of a cDNA by standard PCR methods or for the design of a hybridization probe that can be used to screen a cDNA library using standard hybridization methods. Another preferred stimulatory compound is a nucleic acid molecule encoding the spliced form of XBP-1.

Following isolation or amplification of XBP-1 cDNA or cDNA encoding a molecule in a signal transduction pathway involving XBP-1, the DNA fragment is introduced into a suitable expression vector, as described above. For example, nucleic acid molecules encoding XBP-1 in the form suitable for expression of the XBP-1 in a host cell, can be prepared as described above using nucleotide sequences known in the art. The nucleotide sequences can be used for the design of PCR primers that allow for amplification of a CDNA by standard PCR methods or for the design of a hybridization probe that can be used to screen a cDNA library using standard hybridization methods.

In one embodiment, a stimulatory agent can be present in an inducible construct, e.g., as shown in Example 18. In another embodiment, a stimulatory agent can be present in a construct which leads to constitutive expression.

Another form of a stimulatory compound for stimulating expression of XBP-1 or a molecule in a signal transduction pathway involving XBP-1 in a cell is a chemical compound that specifically stimulates the expression, processing, post-translational modification, or activity of endogenous spliced XBP-1. Such compounds can be identified using screening assays that select for compounds that stimulate the expression of XBP-1 that can be spliced or activity of spliced XBP-1 as described herein.

In another embodiment, a stimulatory compound is one that stimulates the expression or activity of a negative regulator of spliced XBP-1, e.g., unspliced XBP-1. For example, in one embodiment, a cell can be engineered to express a form of unspliced XBP-protein that cannot be spliced. For example, as described in the instant examples, an XBP-1 molecule can be engineered such that splicing cannot occur, e.g., by including mutations in the loop region. This molecule can be introduced into cells to inhibit the activity of spliced XBP-1. In another embodiment, an agent can be used to interfere with the degradation of unspliced XBP-1 to thereby increase the concentration of unspliced XBP-1 in a cell. Exemplary agents include proteasome inhibitors. One exemplary proteasome inhibitor is Velcade™. Other proteasome inhibitors are known in the art and can be found, for example, in Kisselev and Goldberg (2001. Chemistry & Biology 8:739) or Lee and Goldberg (1998. Trends in Cell Biology 8:397). In another embodiment, the stability of unspliced XBP-1 can be increased, e.g., by interfering with ubiquitination of unspliced XBP-1.

The methods of modulating XBP-1 signaling (e.g., by modulating the expression and/or activity of XBP-1 or the expression and/or activity of another molecule in a signal transduction pathway involving XBP-1 can be practiced either in vitro or in vivo. For practicing the method in vitro, cells can be obtained from a subject by standard methods and incubated (i.e., cultured) in vitro with a stimulatory or inhibitory compound of the invention to stimulate or inhibit, respectively, the activity of XBP-1. Methods for isolating cells are known in the art.

Cells treated in vitro with either a stimulatory or inhibitory compound can be administered to a subject to influence the biological effects of XBP-1 signaling. For example, cells can be isolated from a subject, expanded in number in vitro and the activity of, e.g., spliced XBP-1, IRE-1, or ATF6α activity in the cells using a stimulatory agent, and then the cells can be readministered to the same subject, or another subject tissue compatible with the donor of the cells. Accordingly, in another embodiment, the modulatory method of the invention comprises culturing cells in vitro with e.g., an XBP-1 modulator or a modulator of a molecule in a signal transduction pathway involving XBP-1 and further comprises administering the cells to a subject. For administration of cells to a subject, it may be preferable to first remove residual compounds in the culture from the cells before administering them to the subject. This can be done for example by gradient centrifugation of the cells or by washing of the tissue. For further discussion of ex vivo genetic modification of cells followed by readministration to a subject, see also U.S. Pat. No. 5,399,346 by W. F. Anderson et al.

In another embodiment, cells can be treated in vitro with e.g., an XBP-1, IRE-1, or ATF6α modulator in order to enhance production of a commercially valuable polypeptide. For example, in one embodiment, production of a polypeptide which is exogenous to a cell can be enhanced. In another embodiment, the polypeptide can be recombinantly expressed by a cell. Exemplary commercially valuable polypeptides include, e.g., immunoglobulins, cytokines, hormones, growth factors, or other polypeptides produced by cells.

In other embodiments, a stimulatory or inhibitory compound is administered to a subject in vivo. Such methods can be used to treat disorders, e.g., as detailed below and/or to increase production of a protein in vivo. For stimulatory or inhibitory agents that comprise nucleic acids (e.g., recombinant expression vectors encoding, e.g., XBP-1, IRE-1, or ATF6α; antisense RNA; intracellular antibodies; or e.g., XBP-1, IRE-1, or ATF6α-derived peptides), the compounds can be introduced into cells of a subject using methods known in the art for introducing nucleic acid (e.g., DNA) into cells in vivo. Examples of such methods include:

Direct Injection: Naked DNA can be introduced into cells in vivo by directly injecting the DNA into the cells (see e.g., Acsadi et al. (1991) Nature 332:815-818; Wolff et al. (1990) Science 247:1465-1468). For example, a delivery apparatus (e.g., a “gene gun”) for injecting DNA into cells in vivo can be used. Such an apparatus is commercially available (e.g., from BioRad).

Receptor-Mediated DNA Uptake: Naked DNA can also be introduced into cells in vivo by complexing the DNA to a cation, such as polylysine, which is coupled to a ligand for a cell-surface receptor (see for example Wu, G. and Wu, C. H. (1988) J. Biol. Chem. 263:14621; Wilson et al. (1992) J. Biol. Chem. 267:963-967; and U.S. Pat. No. 5,166,320). Binding of the DNA-ligand complex to the receptor facilitates uptake of the DNA by receptor-mediated endocytosis. A DNA-ligand complex linked to adenovirus capsids which naturally disrupt endosomes, thereby releasing material into the cytoplasm can be used to avoid degradation of the complex by intracellular lysosomes (see for example Curiel et al. (991) Proc. Natl. Acad. Sci. USA 88:8850; Cristiano et al. (1993) Proc. Natl. Acad. Sci. USA 90:2122-2126).

Retroviruses: Defective retroviruses are well characterized for use in gene transfer for gene therapy purposes (for a review see Miller, A. D. (1990) Blood 76:271). A recombinant retrovirus can be constructed having a nucleotide sequences of interest incorporated into the retroviral genome. Additionally, portions of the retroviral genome can be removed to render the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology. Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines include ψCrip, ψCre, ψ2 and ψAm. Retroviruses have been used to introduce a variety of genes into many different cell types, including epithelial cells, endothelial cells, lymphocytes, myoblasts, hepatocytes, bone marrow cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573). Retroviral vectors require target cell division in order for the retroviral genome (and foreign nucleic acid inserted into it) to be integrated into the host genome to stably introduce nucleic acid into the cell. Thus, it may be necessary to stimulate replication of the target cell.

Adenoviruses: The genome of an adenovirus can be manipulated such that it encodes and expresses a gene product of interest but is inactivated in terms of its ability to replicate in a normal lytic viral life cycle. See for example Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 d1324 or other strains of adenovirus (e.g., Ad2, Ad3, Ad7 etc.) are well known to those skilled in the art. Recombinant adenoviruses are advantageous in that they do not require dividing cells to be effective gene delivery vehicles and can be used to infect a wide variety of cell types, including airway epithelium (Rosenfeld et al. (1992) cited supra), endothelial cells (Lemarchand et al. (1992) Proc. Natl. Acad Sci. USA 89:6482-6486), hepatocytes (Herz and Gerard (1993) Proc. Natl. Acad. Sci. USA 90:2812-2816) and muscle cells (Quantin et al. (1992) Proc. Natl. Acad. Sci. USA 89:2581-2584). Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Ahmand and Graham (1986) J. Virol. 57:267). Most replication-defective adenoviral vectors currently in use are deleted for all or parts of the viral E1 and E3 genes but retain as much as 80% of the adenoviral genetic material.

Adeno-Associated Viruses: Adeno-associated virus (AAV) is a naturally occurring defective virus that requires another virus, such as an adenovirus or a herpes virus, as a helper virus for efficient replication and a productive life cycle. (For a review see Muzyczka et al. Curr. Topics in Micro. and Immunol. (1992) 158:97-129). It is also one of the few viruses that may integrate its DNA into non-dividing cells, and exhibits a high frequency of stable integration (see for example Flotte et al. (1992) Am. J. Respir. Cell. Mol. Biol. 7:349-356; Samulski et al. (1989) J. Virol. 63:3822-3828; and McLaughlin et al. (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al. (1985) Mol. Cell. Biol. 5:3251-3260 can be used to introduce DNA into cells. A variety of nucleic acids have been introduced into different cell types using AAV vectors (see for example Hermonat et al. (1984) Proc. Natl. Acad. Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790).

The efficacy of a particular expression vector system and method of introducing nucleic acid into a cell can be assessed by standard approaches routinely used in the art. For example, DNA introduced into a cell can be detected by a filter hybridization technique (e.g., Southern blotting) and RNA produced by transcription of introduced DNA can be detected, for example, by Northern blotting, RNase protection or reverse transcriptase-polymerase chain reaction (RT-PCR). The gene product can be detected by an appropriate assay, for example by immunological detection of a produced protein, such as with a specific antibody, or by a functional assay to detect a functional activity of the gene product, such as an enzymatic assay.

In one embodiment, if the stimulatory or inhibitory compounds can be administered to a subject as a pharmaceutical composition. In one embodiment, the invention is directed to an active compound (e.g., a modulator of XBP-1 or a molecule in a signal transduction pathway involving XBP-1) and a carrier. Such compositions typically comprise the stimulatory or inhibitory compounds, e.g., as described herein or as identified in a screening assay, e.g., as described herein, and a pharmaceutically acceptable carrier. Pharmaceutically acceptable carriers and methods of administration to a subject are described herein.

In one embodiment, the active compounds of the invention are administered in combination with other agents. For example, in one embodiment, an active compound of the invention, e.g., a compound that modulates an XBP-1 signal transduction pathway (e.g., by directly modulating XBP-1 activity) is administered with another compound known in the art to be useful in treatment of a particular condition or disease. For example, in one embodiment, for the treatment of a malignancy, an active compound of the invention can be administered in combination with a known anti-tumor therapy (e.g., radiation, chemotherapy, and/or a proteasome inhibitor (such as Velcade™). In another embodiment, an active compound of the invention (e.g., a proteasome inhibitor or a compound that directly modulates XBP-1 activity) can be administered or in combination with an agent that induces ER stress in cells (e.g., an agent such as tunicamycin, an agent that modulates Ca++ influx in cells, or an anti-angiogenic factor that increases hypoxia in the cells of a tumor) to treat a malignancy. In another embodiment of the invention, a proteasome inhibitor can be used in combination with an agent that induces ER stress in cells to disrupt the UPR. In one embodiment, treatment of cells with a proteasome inhibitor and an agent that induces ER stress results in apoptosis of the cells.

V. Diagnostic Assays

In another aspect, the invention features a method of diagnosing a subject for a disorder associated with aberrant biological activity or XBP-1 (e.g., that would benefit from modulation of the UPR, modulation of cellular differentiation, modulation of IL-6 production, modulation of immunoglobulin production, modulation of the proteasome pathway, modulation of protein folding and transport, modulation of terminal B cell differentiation, and modulation of apoptosis).

In one embodiment, the invention comprises identifying the subject as one that would benefit from modulation of an XBP-1 activity, e.g., modulation of the UPR. For example, in one embodiment, expression of XBP-1 or a molecule in a signal transduction pathway involving XBP-1 can be detected in cells of a subject suspected of having a disorder associated with aberrant biological activity of XBP-1. The expression of XBP-1 or a molecule in a signal transduction pathway involving XBP-1 in cells of said subject could then be compared to a control and a difference in expression of XBP-1 or a molecule in a signal transduction pathway involving XBP-1 in cells of the subject as compared to the control could be used to diagnose the subject as one that would benefit from modulation of an XBP-1 activity.

The “change in expression” or “difference in expression” of XBP-1 or a molecule in a signal transduction pathway involving XBP-1 in cells of the subject can be, for example, a change in the level of expression of XBP-1 or a molecule in a signal transduction pathway involving XBP-1 in cells of the subject as compared to a previous sample taken from the subject or as compared to a control, which can be detected by assaying levels of, e.g., XBP-1 mRNA, for example, by isolating cells from the subject and determining the level of XBP-1 mRNA expression in the cells by standard methods known in the art, including Northern blot analysis, microarray analysis, reverse-transcriptase PCR analysis and in situ hybridizations. For example, a biological specimen can be obtained from the patient and assayed for, e.g., expression or activity of XBP-1 or a molecule in a signal transduction pathway involving XBP-1. For instance, a PCR assay could be used to measure the level of spliced XBP-1 in a cell of the subject. Exemplary PCR assays for detection of spliced XBP-1 are described in the appended Examples. For instance, PCR primers (5′-ACACGCTTGGGAATGGACAC-3′ (SEQ ID NO: 5) and 5′-CCATGGGAAGATGTTCTGGG-3′) (SEQ ID NO: 6) that encompass the missing sequences in XBP-1s can be used to identify spliced XBP-1. A level of spliced XBP-1 higher or lower than that seen in a control or higher or lower than that previously observed in the patient indicates that the patient would benefit from modulation of a signal transduction pathway involving XPB-1. Alternatively, the level of expression of XBP-1 or a molecule in a signal transduction pathway involving XBP-1 in cells of the subject can be detected by assaying levels of, e.g., XBP-1, for example, by isolating cells from the subject and determining the level of XBP-1 or a molecule in a signal transduction pathway involving XBP-1 protein expression by standard methods known in the art, including Western blot analysis, immunoprecipitations, enzyme linked immunosorbent assays (ELISAs) and immunofluorescence. Antibodies for use in such assays can be made using techniques known in the art and/or as described herein for making intracellular antibodies.

In another embodiment, a change in expression of XBP-1 or a molecule in a signal transduction pathway involving XBP-1 in cells of the subject results from one or more mutations (i.e., alterations from wildtype), e.g., the XBP-1 gene and mRNA leading to one or more mutations (i.e., alterations from wildtype) in the amino acid sequence of the protein. In one embodiment, the mutation(s) leads to a form of the molecule with increased activity (e.g., partial or complete constitutive activity). In another embodiment, the mutation(s) leads to a form of the molecule with decreased activity (e.g., partial or complete inactivity). The mutation(s) may change the level of expression of the molecule for example, increasing or decreasing the level of expression of the molecule in a subject with a disorder. Alternatively, the mutation(s) may change the regulation of the protein, for example, by modulating the interaction of the mutant protein with one or more targets e.g., resulting in a form of XBP-1 that cannot be spliced. Mutations in the nucleotide sequence or amino acid sequences of proteins can be determined using standard techniques for analysis of DNA or protein sequences, for example for DNA or protein sequencing, RFLP analysis, and analysis of single nucleotide or amino acid polymorphisms. For example, in one embodiment, mutations can be detected using highly sensitive PCR approaches using specific primers flanking the nucleic acid sequence of interest. In one embodiment, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) PNAS 91:360-364). This method can include the steps of collecting a sample of cells from a patient, isolating nucleic acid (e.g., genomic, DNA) from the cells of the sample, contacting the nucleic acid sample with one or more primers which specifically amplify a sequence under conditions such that hybridization and amplification of the sequence (if present) occurs, and detecting the presence or absence of an amplification product, or detecting the size of the amplification product and comparing the length to a control sample.

In one embodiment, the complete nucleotide sequence for XBP-1 or a molecule in a signal transduction pathway involving XBP-1 can be determined. Particular techniques have been developed for determining actual sequences in order to study polymorphism in human genes. See, for example, Proc. Natl. Acad. Sci. U.S.A. 85, 544-548 (1988) and Nature 330, 384-386 (1987); Maxim and Gilbert. 1977. PNAS 74:560; Sanger 1977. PNAS 74:5463. In addition, any of a variety of automated sequencing procedures can be utilized when performing diagnostic assays ((1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Restriction fragment length polymorphism mappings (RFLPS) are based on changes at a restriction enzyme site. In one embodiment, polymorphisms from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Moreover, the use of sequence specific ribozymes (see, for example, U.S. Pat. No. 5,498,531) can be used to score for the presence of a specific ribozyme cleavage site.

Another technique for detecting specific polymorphisms in particular DNA segment involves hybridizing DNA segments which are being analyzed (target DNA) with a complimentary, labeled oligonucleotide probe. See Nucl. Acids Res. 9, 879-894 (1981). Since DNA duplexes containing even a single base pair mismatch exhibit high thermal instability, the differential melting temperature can be used to distinguish target DNAs that are perfectly complimentary to the probe from target DNAs that only differ by a single nucleotide. This method has been adapted to detect the presence or absence of a specific restriction site, U.S. Pat. No. 4,683,194. The method involves using an end-labeled oligonucleotide probe spanning a restriction site which is hybridized to a target DNA. The hybridized duplex of DNA is then incubated with the restriction enzyme appropriate for that site. Reformed restriction sites will be cleaved by digestion in the pair of duplexes between the probe and target by using the restriction endonuclease. The specific restriction site is present in the target DNA if shortened probe molecules are detected.

Other methods for detecting polymorphisms in nucleic acid sequences include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes of formed by hybridizing (labeled) RNA or DNA containing the polymorphic sequence with potentially polymorphic RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels. See, for example, Cotton et al. (1988) Proc. Natl Acad Sci USA 85:4397; Saleeba et al. (1992) Methods Enzymol. 217:286-295. In a preferred embodiment, the control DNA or RNA can be labeled for detection.

In another embodiment, alterations in electrophoretic mobility can be used to identify polymorphisms. For example, single strand conformation polymorphism (SSCP) may be used to detect differences in electrophoretic mobility between mutant and wild type nucleic acids (Orita et al. (1989) Proc Natl. Acad. Sci USA: 86:2766, see also Cotton (1993) Mutat Res 285:125-144; and Hayashi (1992) Genet Anal Tech Appl 9:73-79). Single-stranded DNA fragments of sample and control nucleic acids can be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment, the movement of nucleic acid molecule comprising polymorphic sequences in polyacrylamide gels containing a gradient of denaturant is assayed using denaturing gradient gel electrophoresis (DGGE) (Myers et al. (1985) Nature 313:495). When DGGE is used as the method of analysis, DNA can be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting polymorphisms include, but are not limited to, selective oligonucleotide hybridization, selective amplification, or selective primer extension. For example, oligonucleotide primers may be prepared in which the polymorphic region is placed centrally and then hybridized to target DNA under conditions which permit hybridization only if a perfect match is found (Saiki et al. (1986) Nature 324:163); Saiki et al. (1989) Proc. Natl Acad. Sci USA 86:6230). Such allele specific oligonucleotides are hybridized to PCR amplified target DNA or a number of different polymorphisms when the oligonucleotides are attached to the hybridizing membrane and hybridized with labeled target DNA.

Another process for studying differences in DNA structure is the primer extension process which consists of hybridizing a labeled oligonucleotide primer to a template RNA or DNA and then using a DNA polymerase and deoxynucleoside triphosphates to extend the primer to the 5′ end of the template. Resolution of the labeled primer extension product is then done by fractionating on the basis of size, e.g., by electrophoresis via a denaturing polyacrylamide gel. This process is often used to compare homologous DNA segments and to detect differences due to nucleotide insertion or deletion. Differences due to nucleotide substitution are not detected since size is the sole criterion used to characterize the primer extension product.

Another process exploits the fact that the incorporation of some nucleotide analogs into DNA causes an incremental shift of mobility when the DNA is subjected to a size fractionation process, such as electrophoresis. Nucleotide analogs can be used to identify changes since they can cause an electrophoretic mobility shift. See, U.S. Pat. No. 4,879,214.

Many other techniques for identifying and detecting polymorphisms are known to those skilled in the art, including those described in “DNA Markers: Protocols, Applications and Overview,” G. Caetano-Anolles and P. Gresshoff ed., (Wiley-VCH, New York) 1997, which is incorporated herein by reference as if fully set forth.

In addition, many approaches have also been used to specifically detect SNPs. Such techniques are known in the art and many are described e.g., in DNA Markers: Protocols, Applications, and Overviews. 1997. Caetano-Anolles and Gresshoff, Eds. Wiley-VCH, New York, ppl 99-211 and the references contained therein). For example, in one embodiment, a solid phase approach to detecting polymorphisms such as SNPs can be used. For example an oligonucleotide ligation assay (OLA) can be used. This assay is based on the ability of DNA ligase to distinguish single nucleotide differences at positions complementary to the termini of co-terminal probing oligonucleotides (see, e.g., Nickerson et al. 1990. Proc. Natl. Acad. Sci. USA 87:8923. A modification of this approach, termed coupled amplification and oligonucleotide ligation (CAL) analysis, has been used for multiplexed genetic typing (see, e.g., Eggerding 1995 PCR Methods Appl. 4:337); Eggerding et al. 1995 Hum. Mutat. 5:153).

In another embodiment, genetic bit analysis (GBA) can be used to detect a SNP (see, e.g., Nikiforov et al. 1994. Nucleic Acids Res. 22:4167; Nikiforov et al. 1994. PCR Methods Appl. 3:285; Nikiforov et al. 1995. Anal Biochem. 227:201). In another embodiment, microchip electrophoresis can be used for high-speed SNP detection (see e.g., Schmalzing et al. 2000. Nucleic Acids Research, 28). In another embodiment, matrix-assisted laser desorption/ionization time-of-flight mass (MALDI TOF) mass spectrometry can be used to detect SNPs (see, e.g., Stoerker et al. Nature Biotechnology 18:1213).

In another embodiment, a difference in a biological activity of XBP-1 between a subject and a control can be detected. For example, an activity of XBP-1 or a molecule in a signal transduction pathway involving XBP-1 can be detected in cells of a subject suspected of having a disorder associated with aberrant biological activity of XBP-1. The activity of XBP-1 or a molecule in a signal transduction pathway involving XBP-1α in cells of the subject could then be compared to a control and a difference in activity of XBP-1 or a molecule in a signal transduction pathway involving XBP-1 in cells of the subject as compared to the control could be used to diagnose the subject as one that would benefit from modulation of an XBP-1 activity. Activities of XBP-1 or molecules in a signal transduction pathway involving XBP-1 can be detected using methods described herein or known in the art.

In preferred embodiments, the diagnostic assay is conducted on a biological sample from the subject, such as a cell sample or a tissue section (for example, a freeze-dried or fresh frozen section of tissue removed from a subject). In another embodiment, the level of expression of XBP-1 or a molecule in a signal transduction pathway involving XBP-1 in cells of the subject can be detected in vivo, using an appropriate imaging method, such as using a radiolabeled antibody.

In one embodiment, the level of expression of XBP-1 or a molecule in a signal transduction pathway involving XBP-1 in cells of the test subject may be elevated (i.e., increased) relative to the control not associated with the disorder or the subject may express a constitutively active (partially or completely) form of the molecule. This elevated expression level of, e.g., XBP-1 or expression of a constitutively active form of spliced XBP-1, can be used to diagnose a subject for a disorder associated with increased XBP-1 activity.

In another embodiment, the level of expression of XBP-1 or a molecule in a signal transduction pathway involving XBP-1 in cells of the subject may be reduced (i.e., decreased) relative to the control not associated with the disorder or the subject may express an inactive (partially or completely) mutant form of, e.g., spliced XBP-1. This reduced expression level of spliced XBP-1 or expression of an inactive mutant form of spliced XBP-1 can be used to diagnose a subject for a disorder, such as immunodeficiency disorders characterized by insufficient antibody production.

In one embodiment, the level of expression of gene whose expression is regulated by XBP-1 can be measured (e.g., ERdj4, p58^{IPK}, EDEM, PDI-P5, RAMP4, BiP, XBP-1, or ATF6α).

In another embodiment, an assay diagnosing a subject as one that would benefit from modulation of XBP-1 expression, processing, post-translational modification, and/or activity (or a molecule in a signal transduction pathway involving XBP-1 is performed prior to treatment of the subject.

The methods described herein may be performed, for example, by utilizing pre-packaged diagnostic kits comprising at least one probe/primer nucleic acid or other reagent (e.g., antibody), which may be conveniently used, e.g., in clinical settings to diagnose patients exhibiting symptoms or family history of a disease or illness involving XBP-1 or a molecule in a signal transduction pathway involving XBP-1.

VI. Kits of the Invention

Another aspect of the invention pertains to kits for carrying out the screening assays, modulatory methods or diagnostic assays of the invention. For example, a kit for carrying out a screening assay of the invention can include an indicator composition comprising XBP-1 or a molecule in a signal transduction pathway involving XBP-1, means for measuring a readout (e.g., protein secretion) and instructions for using the kit to identify modulators of biological effects of XBP-1. In another embodiment, a kit for carrying out a screening assay of the invention can include cells deficient in XBP-1 or a molecule in a signal transduction pathway involving XBP-1, means for measuring the readout and instructions for using the kit to identify modulators of a biological effect of XBP-1.

In another embodiment, the invention provides a kit for carrying out a modulatory method of the invention. The kit can include, for example, a modulatory agent of the invention (e.g., XBP-1 inhibitory or stimulatory agent) in a suitable carrier and packaged in a suitable container with instructions for use of the modulator to modulate a biological effect of XBP-1.

Another aspect of the invention pertains to a kit for diagnosing a disorder associated with a biological activity of XBP-1 in a subject. The kit can include a reagent for determining expression of XBP-1 (e.g., a nucleic acid probe for detecting XBP-1 mRNA or an antibody for detection of XBP-1 protein), a control to which the results of the subject are compared, and instructions for using the kit for diagnostic purposes.

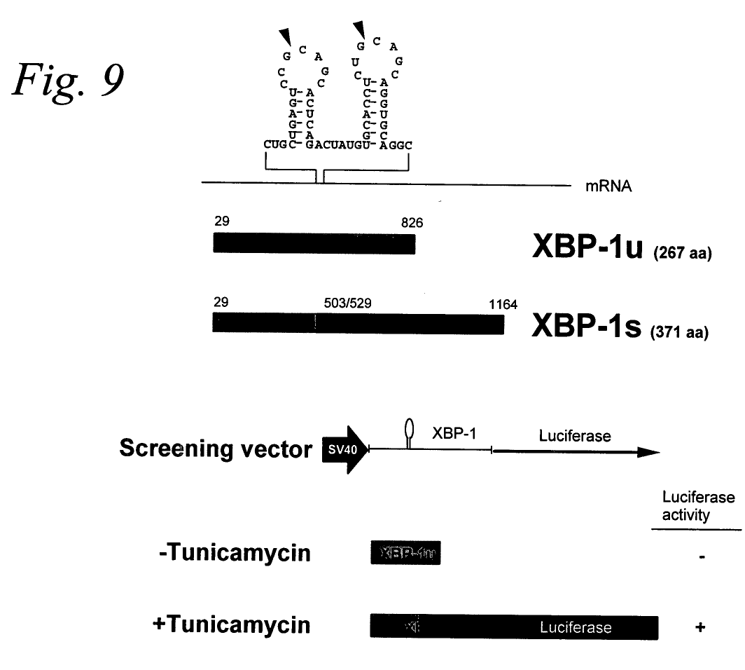

Another aspect of the invention pertains to methods of detecting splicing of XBP-1 and kits for performing such methods. Such methods are useful in identifying agents that modulate splicing. The invention also pertains to constructs comprising XBP-1 or a portion thereof (e.g., the splice region of XBP-1 and a transcriptional activating domain of XBP-1). In one embodiment, such a construct comprises a transactivation domain of XBP-1 (Clauss et al. 1996. Nucleic Acids Research 24:1855). Cells can be engineered to express such constructs and a reporter gene operably linked to a regulatory region responsive to spliced XBP-1. In one embodiment, a cell is engineered to express a screening vector comprising XBP-1 linked to a reporter gene (e.g., luciferase) such that when the spliced form of XBP-1 is made, the reporter gene is transcribed and when the unspliced form of XBP-1 is made, the reporter gene is not transcribed (see the schema presented in FIG. 9). In one embodiment, such an assay can be performed in the presence and absence of a compound that promotes the unfolded protein response, e.g., tunicamycin, so that the role of a test compound on that response can be measured (e.g., the ability of the compound to up or downmodulate this response can be tested). In one embodiment, the cell can further express an exogenous or an endogenous IRE-1 molecule. Test compounds can be identified as stimulators or inhibitors of XBP-1 splicing by comparing the amount of XBP-1 splicing in the presence and the absence of the test compound. In one embodiment, the invention also pertains to a kit for detecting splicing of XBP-1. The kit can include a recombinant cell comprising an exogenous XBP-1 molecule or a portion thereof, and a reporter gene operably linked to a regulatory region responsive to XBP-1 such that upon splicing of the XBP-1 protein, transcription of the reporter gene occurs.

VII Immunomodulatory Compositions

Agents that modulate XBP-1 activity, expression, processing, post-translational modifications, or activity, expression, processing, post-translational modification of one or more molecules in a signal transduction pathway involving XBP-1 are also appropriate for use in immunomodulatory compositions. Stimulatory or inhibitory agents of the invention can be used to up or down regulate the immune response in a subject. In preferred embodiments, the humoral immune response is regulated.

The modulating agents of the invention can be given alone, or in combination with an antigen to which an enhanced immune response or a reduced immune response is desired.

In one embodiment, agents which are known adjuvants can be administered with the subject modulating agents. At this time, the only adjuvant widely used in humans has been alum (aluminum phosphate or aluminum hydroxide). Saponin and its purified component Quil A, Freund's complete adjuvant and other adjuvants used in research and veterinary applications have potential use in human vaccines.

However, new chemically defined preparations such as muramyl dipeptide, monophosphoryl lipid A, phospholipid conjugates such as those described by Goodman-Snitkoffet al. J. Immunol. 147:410-415 (1991) resorcinols, non-ionic surfactants such as polyoxyethylene oleyl ether and n-hexadecyl polyethylene ether, enzyme inhibitors include pancreatic trypsin inhibitor, diisopropylfluorophosphate (DEP) and trasylol can also be used. In embodiments in which antigen is administered, the antigen can e.g., be encapsulated within a proteoliposome as described by Miller et al., J. Exp. Med. 176:1739-1744 (1992) and incorporated by reference herein, or in lipid vesicles, such as Novasome TM lipid vesicles (Micro Vescular Systems, Inc.,Nashua, N.H.), to further enhance immune responses.

In one embodiment, a nucleic acid molecule encoding XBP-1 or a molecule in a signal transduction pathway involving XBP-1 or portion thereof is administered as a DNA vaccine. This can be done using a plasmid DNA construct which is similar to those used for delivery of reporter or therapeutic genes. Such a construct preferably comprises a bacterial origin of replication that allows amplification of large quantities of the plasmid DNA; a prokaryotic selectable marker gene; a nucleic acid sequence encoding an, e.g., XBP-1, IRE-1, or ATF6α polypeptide or portion thereof; eukaryotic transcription regulatory elements to direct gene expression in the host cell; and a polyadenylation sequence to ensure appropriate termination of the expressed mRNA (Davis. 1997. Curr. Opin. Biotechnol. 8:635). Vectors used for DNA lmmunization may optionally comprise a signal sequence (Michel et al. 1995. Proc. Natl. Acad. Sci USA. 92:5307; Donnelly et al. 1996. J. Infect Dis. 173:314). DNA vaccines can be administered by a variety of means, for example, by injection (e.g, intramuscular, intradermal, or the biolistic injection of DNA-coated gold particles into the epidermis with a gene gun that uses a particle accelerator or a compressed gas to inject the particles into the skin (Haynes et al. 1996. J. Biotechnol. 44:37)). Alternatively, DNA vaccines can be administered by non-invasive means. For example, pure or lipid-formulated DNA can be delivered to the respiratory system or targeted elsewhere, e.g., Peyers patches by oral delivery of DNA (Schubbert. 1997. Proc. Natl. Acad. Sci. USA 94:961). Attenuated microorganisms can be used for delivery to mucosal surfaces. (Sizemore et al. 1995. Science. 270:29)

In one embodiment, plasmids for DNA vaccination can express XBP-1 (or e.g., IRE-1, or ATF6α) as well as the antigen against which the immune response is desired or can encode modulators of immune responses such as lymphokine genes or costimulatory molecules (Iwasaki et al. 1997. J. Immunol. 158:4591).

This invention is further illustrated by the following examples which should not be construed as limiting. The contents of all references, patents and published patent applications cited throughout this application are hereby incorporated by reference.

### EXAMPLES

The following materials and methods were used in Examples 1-7:

Mice

BALB/c and 129S6 mice were obtained from the Jackson Laboratory (Bar Harbor, Me.) and Taconic (Germantown, N.Y.), respectively. STAT6-deficient mice were used at 6-8 weeks of age and were maintained in pathogen-free facilities in accordance with the guidelines of the Committee on Animals of Harvard Medical School.

Cell Culture and Cell Lines

Mouse splenocytes or purified B cells (purified mature B cells were isolated from spleen and lymph nodes by magnetic CD43 depletion or B220 magnetic bead selection, Miltenyi Biotech, Auburn Calif.) were plated at 1×10⁶ cells/ml in complete media containing RPMI 1640 supplemented with 10% fetal calf serum (FBS)(Hyclone Laboratories), glutamine (2 mM), penicillin (50 units/ml), streptomycin (50 μg/ml), Hepes (100 mM), nonessential amino acids (1×) sodium pyruvate (1 mM) and β-ME (50 μM) and stimulated with anti-CD40(1 μg/ml)(Pharmigen) or LPS (20 μg/ml)(Sigma). Cytokines (R&D Systems, Minneapolis, Minn.) were used at IL-4 (20 ng/ml), IL-2 (20 ng/ml), Il-5 (20 ng/ml), IL-6 (20 ng/ml ), IL-10 (20 ng/ml ) and IL-13 (20 ng/ml). Stimulated B cells were split to a cell concentration of 1×10⁶ cells/ml with fresh media every 24 h. The BCL1 (CWS13.20-3B3 ATCC CRL 1699) cell line was cultured in RPMI media supplemented with 10% FBS, gentamicin (20 μg/ml), and β-ME (50 μM). To induce differentiation cells were plated at 2×10⁵ cells/ml and treated with recombinant mouse IL-2 and IL-5 (20 ng/ml) (R&D systems).

Plasmid Construction and Transient Transfection

The XBP-1 cDNAs for the normal unspliced, but capable of being spliced (XBP-1u/s) and the spliced (XBP-1s) forms were PCR-amplified from the total RNA of untreated and tunicamycin treated NIH3T3 cells, respectively. XBP-1u (unspliced) form was generated by a PCR-based mutagenesis from XBP-unspliced/s cDNA so that the two G residues at position 532 and 535 were changed to A without changing amino acid sequences in XBP-1u open reading frame. Numbers are based on the sequences from gene bank database (NM_{—}013842). These XBP-1 cDNAs were inserted into the pCDNA3.1 plasmid between Hind III and Apa I sites to generate each mammalian expression plasmids. To generate retroviral vectors for each XBP-1 u, XBP-1 u/s and XBP-1s, cDNAs were excised from pCDNA3.1-derived vectors by Pme I digestion and then inserted into the GFP-RV retroviral vector by using blunt end ligation (Sambrook et al., 1989). NIH3T3 cells were transfected by using the Lipofectamine2000 reagents as recommended by the manufacturer.

Northern Hybridization and RT-PCR

Total RNA was isolated using TriZo1 (Gibco-BRL) or Qiashedder/Rneasy RNA purification columns (Quiagen). Northern Blots were performed as described (Rengarajan et al., 2000 Immunity. 12:293-300). Briefly, 7-10 μg RNA were electrophoresed on 1.2% agarose, 6% formaldehyde gels transferred onto Genescreen membrane (NEN) and covalently bound to the membrane using UV Stratalinker (Stratagene). The following probes were used after ³²P-radiolabeling with the ReadyPrime labeling system (Amersham-Pharmacia): XBP-1 (15-830 of the murine coding region), GRP94 and GRP78 (both a kind gift of R. J. Kaufman Univ. of Michigan (Lee et al., 2002)), Blimp (Nco1-Sca1 fragment), c-myc cDNA and Il-6 cDNA . Probe hybridization was performed with Ultrahyb buffer as recommended by the manufacturer (Ambion). Total RNAs were used for the first-strand synthesis with the Superscript reverse transcriptase (Invitrogen). A pair of PCR primers (5′-ACACGCTTGGGAATGGACAC-3′ (SEQ ID NO: 5) and 5′-CCATGGGAAGATGTTCTGGG-3′) (SEQ ID NO: 6) that encompasses the missing sequences in XBP-1s was used for the PCR amplification with AmpliTaq Gold polymerase (Applied Biosystem). PCR products were electrophoresed on a 3% agarose gel (Agarose-1000 Invitro gen) and visualized by ethidium bromide staining.

ELISA Assays.

Assays to measure immunoglobulin or cytokine levels in culture supernatants were performed as described (e.g., Hodge et al., 1996 Immunity. 4:397-405 or Science. 1996 274:1903-5).

Retroviral Transduction of B Cells.

The XBP-1 cDNAs for the unspliced (XBP-1u/s) and the spliced (XBP-1s) forms were PCR-amplified from the total RNA of untreated and tunicamycin treated NIH3T3 cells, respectively, with a primer set (5′-GACGTTTCCTGGCTATGGTGG-3′ (SEQ ID NO: 7) and 5′-CAGGCCTATGCTATCCTCTAGGC-3′) (SEQ ID NO: 8). XBP-1u form was generated by a PCR-based mutagenesis from XBP-unspliced/s cDNA so that the two G residues at position 532 and 535 were changed to A without changing amino acid sequences in the XBP-1u open reading frame. Numbers are based on the sequences from gene bank database (NM_{—}013842 [gi:13775155]). These XBP-1 cDNAs were inserted into the GFP-RV retroviral vector by using blunt end ligation (Sambrook et al., 1989). EFFECTENE transfection (Quiagen) was used to introduce the DNA into the Phoenix cell line as described by manufacturer (e.g., Reimold et al., 2001 Nature. 412:300-7 or Int Immunol. 2001 13:241-8). Viral supernatants were harvested after 48 hours and frozen at −80° C. for later use. B cells were purified from mouse spleens using CD43 depletion or positive selection with B220+ magnetic beads as described by the manufacturer (MidiMacs, Miltenyi Biotech). B cell purity was generally near 95% as confirmed by flow cytometry. The B cells (10⁶ cells /ml) were then activated in culture with LPS 10 μg/ml and F (ab′)₂ anti-IgM (5 μg/ml Southern Biotechnology Associates Inc.) for 24 hours. The 1 ml of activated B cells (10⁶ cells/ml) were mixed with 4 μg of polybrene and 1 ml of virus-containing supernatant and spun at 1000 g for 45 minutes at 24° C. Cells were incubated for 24-36 h at 37° C. and then GFP+ cells were flow sorted and returned to culture with and without stimulation.

### Example 1

### Rapid Induction of XBP-1 by IL-4 in Primary B Cells is STAT-6 Dependent

Since XBP-1 is required for terminal B cell differentiation, it was important to identify the stimuli that regulate XBP-1 gene expression. A plethora of cytokines (IL-2, IL-4, IL-5, IL-6, IL-10, IL-13) has been implicated in plasma cell differentiation both in vitro and in vivo (reviewed in Calame, K. L. (2001 Nat. Inimunol. 2: 1103-1108; Liu, Y. J., and Banchereau, J. (1997). Sem Immunol 9: 235; Zubler, 1997Sem. Hematol 34:13). It was possible that these cytokines effected terminal B cell differentiation by modulating XBP-1 gene expression. Thus, the ability of these cytokines to upregulate XBP-1 mRNA transcripts in B cells was tested. Purified splenic B cells were cultured for 24 h in the presence or absence of various cytokines and XBP-1 expression determined by Northern blot analysis. In FIG. 1(A) B220+ splenic B cells were cultured in the presence of the indicated cytokines at 20 ng/ml for 24 hours. XBP-1 mRNA levels were determined by Northern blot analysis with γ-actin as control. FIG. 1(B) shows results where splenic B cells were treated with recombinant IL-4 for various times, and XBP-1 mRNA levels were determined by Northern blot analysis. In panel (C) Splenic B cells were taken from either normal BALB/c mice, IL-4 or STAT6-deficient mice and then stimulated with recombinant IL-4 for 18 hours. XBP-1 mRNA levels were determined as above. In panel (D) splenic B cells were stimulated with recombinant IL-4 for 4 hours in the absence or presence of 20 μg/ml cycloheximide. Cycloheximide was added to the cells 30 min before the stimulation with IL-4.

FIG. 1A shows that IL-4 alone induced a significant amount of XBP-1 ranscript 24 h after cytokine treatment while the other cytokines tested were inert when ompared to untreated cells. Inclusion of these latter cytokines in IL-4 treated cultures did not result in a further increase of XBP-1 transcripts beyond that observed with IL-4 alone. To more precisely define the kinetics of this upregulation, purified B cell cultures were treated with IL-4 and assayed at intervals up to 24 h. XBP-1 transcripts increased rapidly in response to IL-4, being apparent within 1 h of IL-4 treatment and peaking at around 8 h (FIG. 1B). To determine whether this induction required the synthesis of new proteins, cultures were treated with IL-4 in the presence or absence of the protein synthesis inhibitor, cycloheximide (CHX). The inclusion of cycloheximide did not affect the upregulation of XBP-1 mRNA in response to IL-4 (FIG. 1C). This finding suggested that the rapid upregulation of XBP-1 mRNA in IL-4-treated B cells may depend upon its direct transcriptional activation by the IL-4R linked signaling protein, Stat6. To test this hypothesis, B cells from STAT6^{+/+}, IL4^{-/-} and STAT6^{-/-} mice were treated with IL-4. Control B cells (STAT6^{+/+}) and IL4^{-/-} B cells stimulated for 18 h significantly upregulated XBP-1 transcripts while STAT6^{-/-} treated B cells were unable to induce XBP-1 mRNA (FIG. 1D). Thus, levels of XBP-1 mRNA in B cells are controlled through an IL-4-driven, Stat6-dependent pathway.

### Example 2

### XBP-1 Splicing Correlates with Differentiation of Primary B Cells into Antibody Secreting Cells

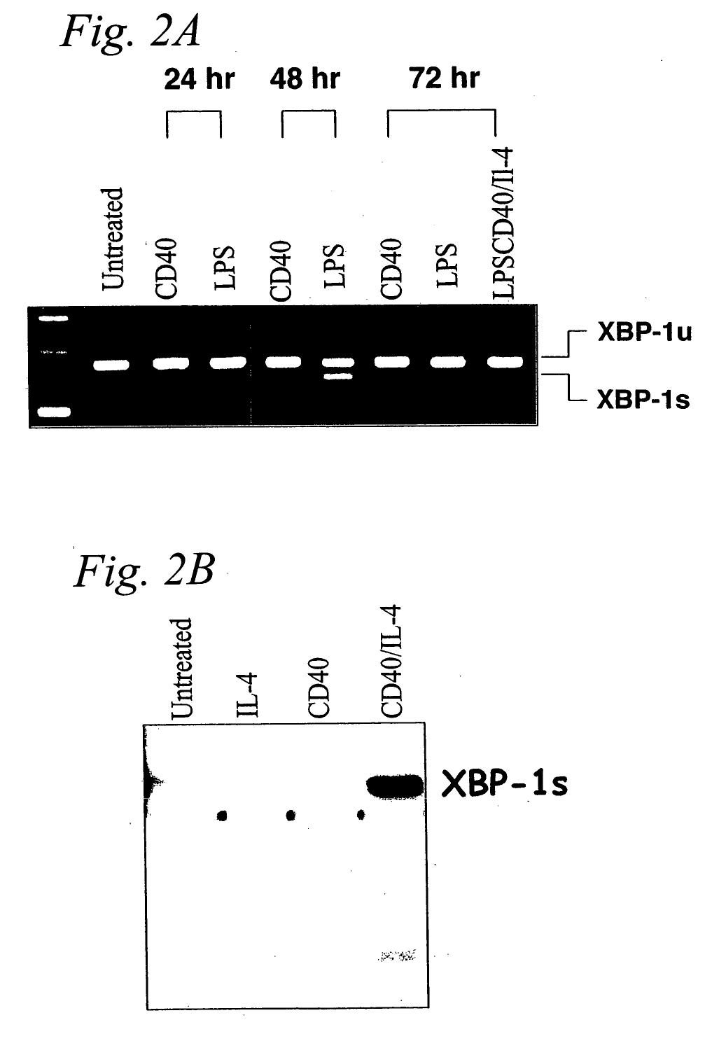

The UPR is induced in cells that detect irregular amounts of unfolded or unassembled protein in the lumen of the ER. Prior to secretion in activated B cells, the increased load of Ig in the ER could be an effective signal to activate IRE-1α and subsequent XBP-1 splicing. To investigate this possibility, the ability of various stimuli to induce XBP-1 splicing was examined. It is well established that stimulation through the CD40 receptor or with mitogens such as lipopolysaccharide (LPS) induces activation and differentiation of murine B cells. Indeed the specific interaction of the CD40 cytoplasmic domain with TRAF6 has recently been shown to be required for plasma cell differentiation (Ahonen et al., 2002 Nat. Immunol. 3: 451-456). In FIG. 2 panel (A) splenic B cells were cultured in the presence of LPS at 20 ng/ml for three days. Total RNAs were prepared at the indicated time point and XBP-1 mRNA levels (a) were determined by Northern blot analysis with γ-actin control (b). RT-PCR analysis was performed with a primer set flanking the spliced-out region in XBP-1s mRNA. PCR products were resolved on 3% agarose gel to separate the bands for the unspliced and spliced XBP-1 mRNA (c). XBP-1s protein levels were also measured by Western blot analysis (d). In panel (B) splenic B cells were stimulated with either IL-4, anti-CD40 or both for the indicated time. RT-PCR analysis was performed as above. In panel (C) XBP-1s protein levels were measured by Western blot analysis in the cells stimulated as indicated for 72 hours.

Both anti-CD40 and LPS cause the upregulation of transcripts encoding XBP-1 in purified murine B cells (Reimold et al., 2001 Nature 412: 300-307) (FIG. 2A), but it was not established whether these transcripts encoded the unspliced or spliced form of XBP-1. Polymerase chain reaction was carried out with reverse transcription (RT-PCR) analysis using MRNA from purified murine B cells treated in vitro with LPS, anti-CD40 or anti-IgM with and without IL-4. Primer sets were used at positions 410 and 580 of murine XBP-1 in order to amplify the region that encompasses the splice junction. RT-PCR analysis from untreated cells and from all groups treated for 24 h revealed a predominant amplified fragment of 171 bp corresponding to unspliced mRNA. The same analysis on samples treated for 48 and 72 h with LPS and anti-CD40 plus IL-4, both effective stimuli for differentiation, revealed the unspliced band of 171 bp and an additional band of 145 bp corresponding to the spliced form of XBP-1 mRNA that lacks 26 bp within this region. Spliced XBP-1 mRNA also appeared 48 and 72 h after anti-CD40 alone (FIG. 2AB). Consistent with its inability to induce differentiation, stimulation through the BCR alone was also unable to induce splicing. Lastly, treatment with exogenous IL-4 that dramatically induced XBP-1 transcripts within 8 h was unable to induce splicing after 24 h of cytokine treatment (FIG. 2B). These results confirm an earlier report that described the production of XBP-1 spliced protein upon LPS treatment (Calfon et al., 2002 Nature 415: 92-96), and extend it to implicate the physiologically relevant signaling pathway, CD40, in the splicing event. However, although anti-CD40 was able to induce the production of the spliced XBP-1 transcript, the production of maximal amounts of spliced XBP-1 protein required stimulation with both IL-4 and CD40, similar to what has been found to be required for Ig production from B cells (FIG. 2C). Similarly, LPS treatment, which can by itself promote Ig production and plasma cell differentiation, was competent to produce substantial amounts of spliced XBP-1 protein in the absence of other stimuli (FIG. 2A). Therefore, the ability of a given stimulus to effect XBP-1 splicing correlates with its ability to promote B cell differentiation.

### Example 3

### XBP-1 Splicing Occurs During Terminal B Cell Differentiation and Correlates with the Induction of the UPR in the BCL-1 Model System

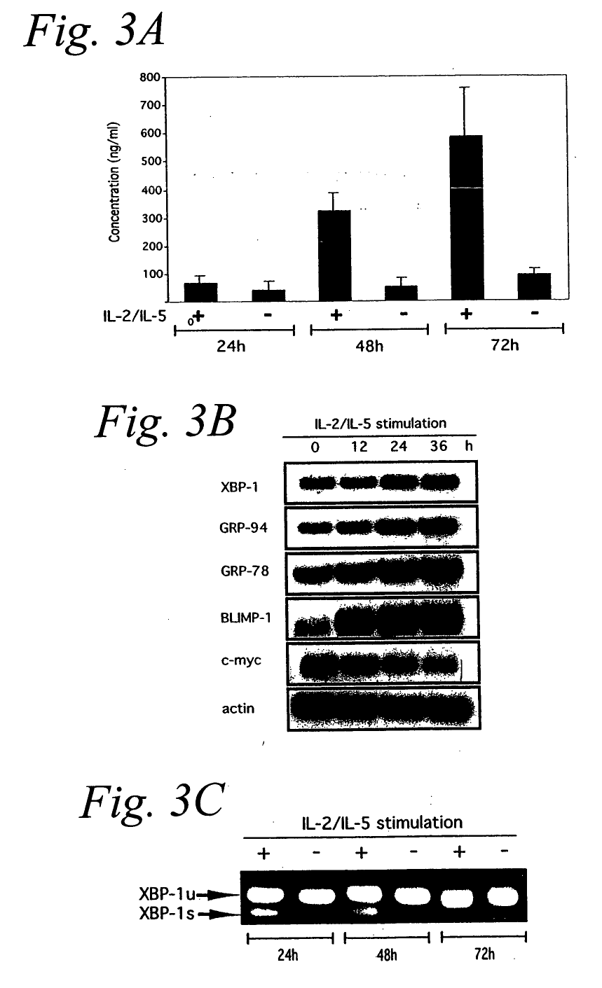

The above experiments explored the control of XBP-1 splicing in the activated B cell. To investigate the regulation of XBP-1 splicing in the last stage of terminal B cell differentiation, the BCI-1 cell line model for plasma cell differentiation was utilized. Upon stimulation with IL-2 and IL-5, this mature B cell line differentiates into an early plasma cell state as evidenced by surface expression of Syndecan (CD 138) and secretion of small amounts of IgM (Blackman et al., 1986 Cell 47: 609-617; Matsui et al., 1989 J Immunol 142: 2918-2923). BCL-1 cells express XBP-1 mRNA at baseline levels and that upon stimulation with IL-2 and IL-5, these levels do not increase (Reimold et al., 2001, supra). To investigate whether XBP-1 splicing occurred during this differentiation process BCL-1 cells were treated with IL-2 and IL-5. In FIG. 3 panel (A) IgM production was measured by ELISA from culture supernatants of BCL-1 cells stimulated with IL-2 and IL-5 (20 ng/ml each) and control unstimulated cells for the times indicated. Experiments were done at least three times and standard deviation is shown. In panel (B) Northern blot analysis containing 7 ug of total RNA from BCL-1 cells stimulated with IL-2 and IL-5 (20 ng/ml each) for 12, 24 and 36 h intervals. In panel (C) Total RNA from BCL-1 cells unstimulated and stimulated with IL-2 and IL-5 (20 ng/ml each) for 24 and 48 h intervals was used for RT-PCR analysis. Primers spanning the splice junction for murine XBP-1 were used to amplify products of unspliced and spliced mRNA. PCR products were electrophoresed on a 3% agarose gel and visualized by ethidium bromide staining.

Verification of differentiation was documented by increased Ig production (FIG. 3A) and increases in the forward versus side scatter parametersin flow cytometry. Northern blot analysis revealed further evidence of differentiation by virtue of a rapid upregulation of BLIMP-1 mRNA with a concomitant repression of cMyc mRNA (FIG. 3B) (Lin et al., 1997 Science 276: 596-599; Turner et al., 1994 Cell 77: 297-306). RT-PCR analysis was carried out using mRNA from BCL-1 cells that had been left untreated or were treated with IL-2 and IL-5. Primer sets were used at positions 410 and 580 of murine XBP-1 in order to amplify the region that encompasses the splice junction as described above. RT-PCR analysis from untreated cells revealed an amplified fragment of 171 bp corresponding to unspliced mRNA. In contrast, the same analysis of mRNA from IL-2 and IL-5 treated cells at 24 and 48 h revealed both the unspliced fragment of 171 bp and an additional band of 145 bp corresponding to the spliced form of XBP-1 mRNA that lacks 26 bp within this region (FIG. 3C). It has been recently shown that overexpression of the spliced form of XBP-1, but not the unspliced form, results in significant induction of UPR reporter constructs in HeLa cells (Yoshida et al., 2001 J Biol Chem 273: 33741-33749). To establish a link between the splicing event, plasma cell differentiation and the UPR, the induction of UPR targets GRP78 and GRP94 in relation to XBP-1 splicing during differentiation in BCI-1 cells was examined. Terminal differentiation of BCL-1 cells was accompanied by significant upregulation of endogenous levels of mRNA encoding the UPR chaperone genes, GRP94 and GRP78, an induction that correlated with the splicing of XBP-1 (FIG. 3B). Therefore, induction of the UPR and subsequent transcriptional activation by the spliced form of XBP-1 are involved in terminal B cell differentiation.

### Example 4

### Overexpression of XBP-1 Enhances IgM Secretion by BCL-1 Cells

Ectopic Expression of XBP-1 in BCl-1 Cells Increases Membrane

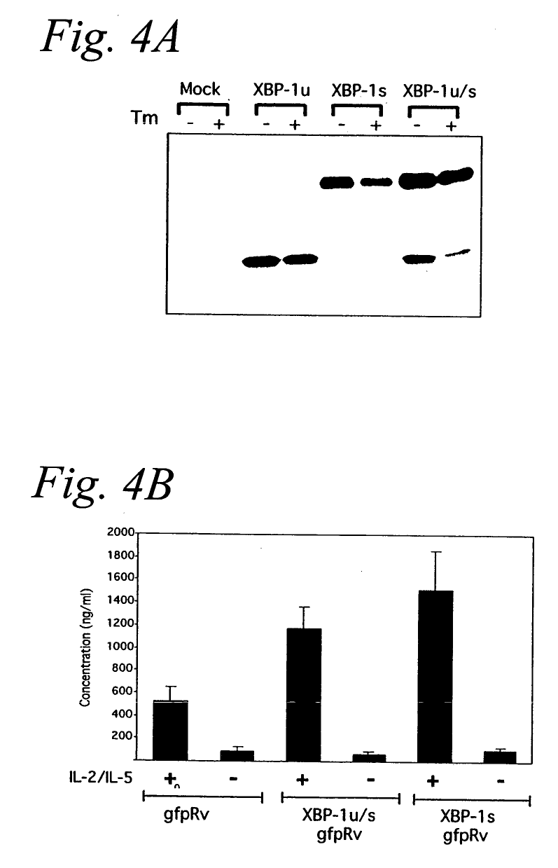

Syndecan-1 levels (Reimold et al., 2001, supra). To determine the effect of unspliced and spliced forms of XBP-1 in IgM production, two bicistronic retroviral vectors were generated, the first expressing both the unspliced and spliced versions of murine XBP-1 and the second encoding only the spliced version of the XBP-1 protein. The first vector, XBP-1u/s, directs expression of XBP-1 mRNA that, when translated, produces the unspliced form and the spliced form upon IRE-dependent splicing. Similar to human XBP-1 mRNA, splicing of murine XBP-1 mRNA results in the excision of 26 bp and a resulting frameshift causes removal of 108 C-terminal aa and the addition of 212 aa to the remaining 159 aa N-terminal region. This construct should result in the production of proteins of 267 aa (33K-unspliced) and 371 aa (54K-spliced), respectively. The vector encoding the spliced form of XBP-1, XBP-1s, only results in the production of a protein containing 371 aa (Calfon et al., 2002 Nature 415: 92-96; Yoshida et al., 2001 J Biol Chem 273: 33741-33749). In FIG. 4 panel (A) total cell lysates from transfected NIH3T3 cells from purified B cells unstimulated and stimulated with tunicamycin were used for Western blot analysis. In panel (B) retroviral transduction with GFP alone (GFP-Rv), XBP-1u/s (XBP-1u/s GFP Rv) or XBP-1s (XBP-1s GFP Rv) of BCl-1 cells was performed. 36 h after transduction cells were sorted for GFP and incubated in the presence or absence of IL-2 and IL-5 (20 ng/ml each) for 72 h. IgM production analyzed by ELISA from culture supernatants of BCL-1 cells unstimulated and stimulated cells. Experiments were done at least three times and standard deviation is shown. Overexpression in NIH 3T3 fibroblasts by transient transfection of XBP-1u/s and XBP-1s cDNA confirmed the appropriate production of the unspliced and spliced forms of XBP-1 as evidenced by the detection of 33K and 54K proteins (FIG. 4A). Retroviral gene transduction of the BCL-1 cell line was then performed and the cells were sorted for GFP expression to generate stable populations of cells expressing control vector, XBP-1u/s and XBP-1s. The cells were then treated with IL-2 and IL-5 or left untreated and then assayed for IgM secretion. In GFP positive BCL-1 cells treated with IL-2 and IL-5 for 3 days, expression of XBP-1u/s increased IgM secretion approximately threefold when compared to vector alone controls. Similarly, XBP-1s expression enhanced Ig secretion approximately fourfold versus control in cytokine treated cells. In GFP positive BCL-1 cells that were untreated with cytokine, neither XBP-1u/s nor XBP-1s forms were able to induce Ig secretion (FIG. 4B). These data demonstrate that the spliced version of XBP-1 can drive Ig production and secretion in the presence of stimuli required for terminal plasma cell differentiation.

### Example 5

### Only the Spliced XBP-1 Protein Restores Ig Production in XBP-1^{-/-} Primary B Cells

Although the BCL-1 model has proved useful in studying plasma cell differentiation, it only partially replicates what occurs in primary B cells where treatment with IL-2 and IL-5 alone does not result in plasma cell differentiation. Therefore, an assessment of XBP-1 function in primary B cells was made to take advantage of mice whose lymphoid system lacks XBP-1. XBP-1 deficient B cells produced very little Ig after in vitro stimulation with LPS when compared to wt B cells and that ectopic expression of a cDNA encoding both unspliced and spliced forms of XBP-1 into XBP-1^{-/-} B cells partially restored Ig secretion (Reimold et al., 2001, supra). These results strongly implicated XBP-1 in the production of Ig by primary B cells. To examine the relative contributions of the unspliced and spliced forms of XBP-1 in Ig production a mutant version of XBP-1 was produced similar those described for yeast and human XBP-1 mRNA (Gonzalez et al., 1999 EMBO J 18: 3119-3132; Kawahara et al., 1998 J Biol Chem 273: 1802-1807; Yoshida et al., 2001, supra) that could not be spliced by IRE1α. Ire1p-inediated splicing of HAC-1 mRNA has been extensively studied in yeast. Based upon these studies, the specific nucleotides required for this unconventional splicing event are well defined. Irelp dependent splicing of the 3′ splice site of yeast Hac 1 mRNA has been shown to be dependent upon positions −3, −1, +3 and +4 within the loop structure of seven nucleotides that is targeted for splicing. Mutations of any of the four critical sites resulted in transcripts that were unable to be spliced in response to tunicamycin treatment (induces UPR via inhibition of glycosylation). Accordingly, a point mutation at positions −1 and +3 in the loop structure of murine XBP-1 was created. This vector is defined as XBP-1u. Overexpression of a vector that contained XBP-1u cDNA resulted in the production of a single 33 K protein confirming exclusive production of the unspliced XBP-1 protein.

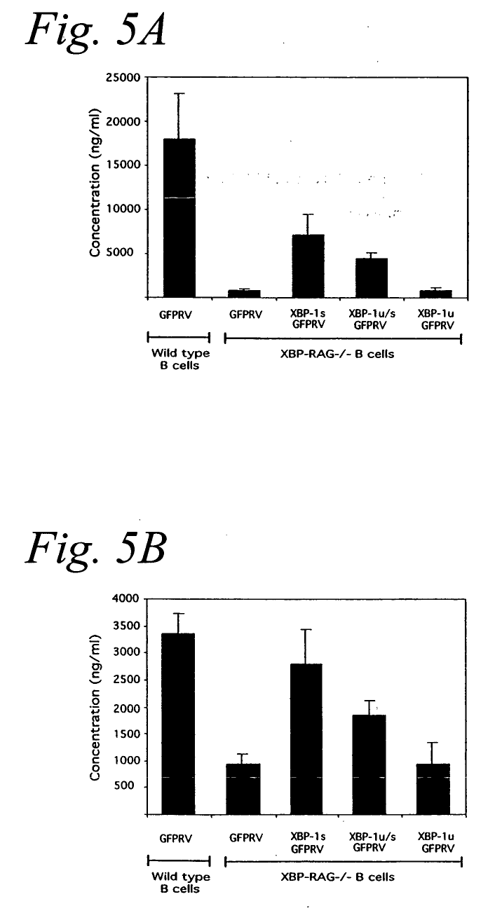

The effects of these three different forms of XBP-1 on Ig secretion were compared in wild type and XBP-1^{-/-} B cells. In vitro activated splenic B cells were transduced using retroviruses expressing bicistronic mRNA encoding XBP-1u/s, XBP-1s, XBP-1u or control GFP. In FIGS. 5(A) and (B) purified B cells from wt and XBP-1^{-/-} mice were activated in culture with LPS 10 μg/ml and F (ab′)₂ anti-IgM (5 μg/ml) for 24 hours. Retroviral transduction with GFP alone (GFP-Rv), XBP-1u/s (XBP-1u/s GFP Rv), XBP-1s (XBP-1s GFP Rv) or XBP-1u (XBP-1u GFP Rv) of activated B cells was performed. Cells were incubated for 24-36 h at 37° C. and then GFP+ cells were flow sorted and then returned to culture with LPS stimulation (10 μg/ml) for 72 h. IgM panel(A) and IgG2b panel (B) production analyzed by ELISA from culture supernatants of stimulated B cells. Experiments were done at least three times and standard deviation is shown.

XBP-1^{-/-}cells were significantly impaired in IgM production as wild type cells expressing control GFP expressed approximately 20-fold more IgM than similar control transduced XBP-1^{-/-} cells. However, expression of XBP-1 unspliced/s or XBP-1s increased the secretion of IgM in XBP-1^{-/-} B cells approximately ten-fold when compared to control cells. In contrast, the expression of the XBP-1u, that exclusively generates the unspliced form of XBP-1, was unable to increase IgM secretion in XBP-1^{-/-} B cells (FIG. 5A).

LPS is known to induce class switching from IgM to IgG2b subclasses upon in vitro stimulation of B cells. IgG2b production by in vitro stimulation with LPS is also significantly reduced in XBP-1^{-/-} B cells. The ability of the two versions of XBP-1 to restore IgG2b production in XBP-1^{-/-}0 B cells was examined. Wild type cells expressing control GFP expressed 5 times more IgG2b than similar control transduced XBP-1^{-/-} cells. Expression of XBP-1 unspliced/s or XBP-1s retroviruses increased the secretion of IgG2b in XBP-1^{-/-} B cells approximately 2-4-fold when compared to control cells. In contrast, the expression of XBP-1u was unable to increase IgG secretion in XBP-1^{-/-} B cells (FIG. 5B).

Thus, only the spliced form of XBP-1, and not the unspliced form, is responsible for and indeed required for, the production of secreted Ig in normal B-lymphocytes. Therefore, the signaling system set in motion by the UPR is required for B cell differentiation.

### Example 6

### The XBP-1 Spliced Protein Induces IL-6 Secretion in wt and XBP-1^{-/-} B Cells

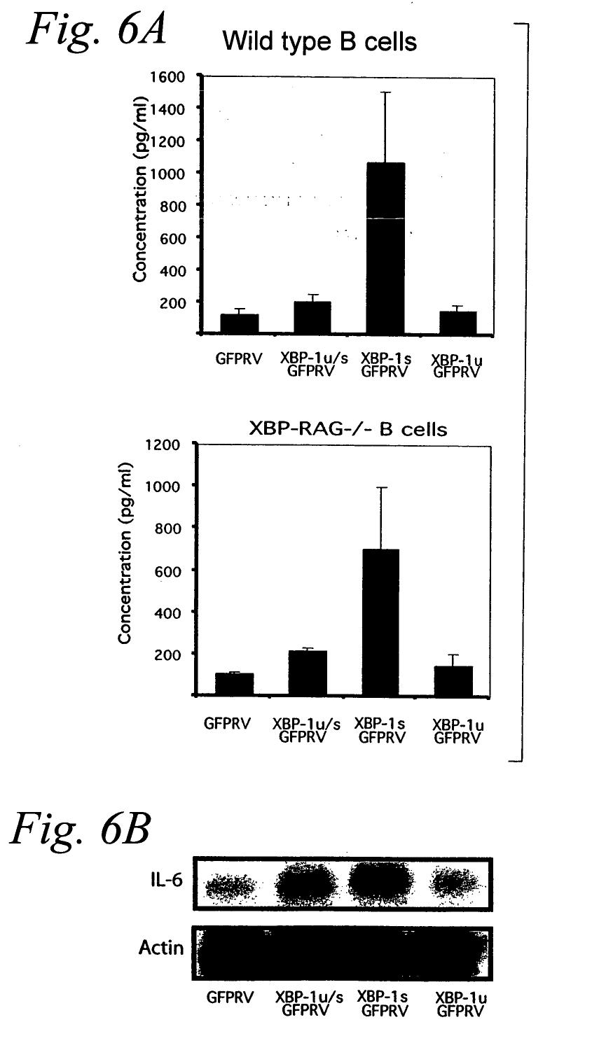

IL-6 has been shown to drive purified B cells into Ig secreting plasma cells and to act as an important growth factor for malignant plasma cells (multiple myeloma cells)(Hallek et al., 1998 Blood 91: 3-21; Hirano and Kishimoto, 1989 Prog Growth Fact Res. 1: 133-142; Kawano et al., 1988 Nature 332: 83-85). In human multiple myeloma cells, XBP-1 was induced by IL-6 treatment and implicated in the proliferation of malignant plasma cells (Wen et al., 1999). However, no role for IL-6 in upregulating XBP-1 transcripts or in mediating splicing of XBP-1 RNA in mature B cells has been described (FIG. 6A). The ability of XBP-1 to induce IL-6 production in wt and XBP-1^{-/-} primary B cells was tested. As described above, in vitro activated murine splenic B cells were transduced using retroviruses expressing bicistronic mRNA for XBP-1u/s, XBP-1s, XBP-1u and control GFP. After cell sorting, GFP⁺ cells were stimulated with LPS and assayed for cytokine production after 72 h. In FIG. 6 panel (A) purified B cells from wt and XBP-1^{-/-} mice were activated in culture with LPS 10 μg/ml and F(ab′)₂ anti-IgM (5 μg/ml ) for 24 hours. Retroviral transduction with GFP alone (GFP-Rv), XBP-1u/s (XBP-1 u/s GFP Rv), XBP-1s (XBP-1s GFP Rv) or XBP-1u (XBP-1u GFP Rv) of activated B cells was performed. Cells were incubated for 24-36 h at 37° C. and then GFP+ cells were flow sorted and then returned to culture with LPS stimulation (10 μg/ml) for 72 h. Cytokine production was analyzed by ELISA from culture supernatants of stimulated B cells. Experiments were done at least three times and standard deviation is shown. In panel (B) Total RNAs were prepared from stimulated B cells above and XBP-1 mRNA levels were determined by Northern blot analysis with γ-actin control

Wild type and XBP-1^{-/-} cells expressing control GFP or XBP-1u retroviruses expressed moderate amounts of IL-6 similar to what has been reported in the literature (Burdin et al., 1995. J Immunol 154: 2533-2544). The expression of XBP-1 u/s increased IL-6 secretion over control approximately 2-fold. Remarkably, the introduction of the XBP-1s form increased the secretion of IL-6 in wild type and XBP-1^{-/-} B cells approximately ten-fold and seven fold respectively when compared to control cells (FIG. 6A). Northern blot analysis of mRNA from XBP-1s transduced cells revealed approximately a 5-fold increase in IL-6 gene expression when compared to controls (FIG. 6B). The levels of other cytokines such as IL-2, IL-4, IL-5 and IL-10 were unaffected. These data suggest that, in addition to its role in the UPR, the spliced form of XBP-1 also acts to regulate the expression of the important plasma cell growth factor, IL-6.

### Example 7

### Unspliced XBP-1 is Ubiquitinated and is Unstable

Tagged XBP-1 spliced and unspliced proteins were co-transfected into NIH 3T3 cells with a tagged ubiquitin expression construct. Lysates were run over a Nickel column to select for ubiquitinated proteins. These proteins were eluted, and western blot analysis performed with anti-XBP-1 antibodies. The results show that only unspliced XBP-1 protein is ubiquinated. Moreover, ³⁵S pulse chase experiments in myeloma cells revealed a very short half life and rapid degradation of unspliced (no band visible after a 60 minute chase) but not spliced XBP-1 protein (band still visible after 120 min chase), consistent with its ubiquitination.

### Example 8

### Proteasome Inhibitors Stabilize Unspliced XBP-1 at the Expense of Spliced XBP-1

A variety of reversible and irreversible inhibitors that inhibit degradation of proteins by the ubiquitin-proteasome pathway have recently been identified. Proteasome inhibitors MG132 and PS341 block the rapid degradation of ubiquitinated proteins such as unspliced, but not spliced XBP-1. Myeloma cells express primarily spliced XBP-1 protein as expected. However, treatment of the mouse myeloma MOPC315 and the human myeloma MM, 1S with MG132 or PS341 results in a dramatic increase in the amount of unspliced XBP-1 protein and a concomitant decrease in levels of the active spliced XBP-1 protein. In this experiment lysates from MOPC315 cells were untreated or treated for 6 hours with 5 ug/ml of tunicamycin or 20 uM of MG-132 and were analyzed for the presence of XBP-1 using XBP-1 antibody. The two forms of XBP-1 were distinguished by their molecular weight.

### Example 9

### Unspliced XBP-1 Protein Inhibits XBP-1 Activity in the Presence of Proteasome Inhibitors

In the presence of proteasome inhibitors, unspliced XBP-1 protein accumulates in the cell and is present at higher levels than the spliced protein. Lysates from Mm. 1S cells were untreated or treated for 1, 4, or 8 hours with 20 uM of PS-341 and were analyzed by western blotting with anti-XBP-1 antibody. Because unspliced, but not spliced XBP-1 is stabilized, the ratio of unspliced to spliced XBP-1 increases in cells treated with proteasome inhibitors.

Transient transfection experiments clearly demonstrate that unspliced XBP-1 protein dramatically represses the transcriptional activating ability of the spliced protein. The fold induction of luciferase was measured in NIH3T3 cells bearing XBP-luciferase. The constructs included four copies of the XBP-1 target sequence TGGATGACGTGTACA (SEQ ID NO: 9) fused to the minimal promoter of the mouse RANTES gene (Clauss et al. Nucleic Acids Research 1996. 24:1855) or five copies of the ATF6/XBP-1 target TCGAGACAGGTGCTGACGTGGCGATTCC (SEQ ID NO: 10) and comprising −53/+45 of the cfos promoter (J. Biol. Chem. 275:27013). In the presence of 5 uM MG-132, the presence of unspliced XBP-1 inhibits the fold induction of luciferase from 41.9 to 5.8. Thus, in the presence of proteasome inhibitors, the unspliced protein blocks the function of the spliced protein.

The following materials and methods were used in Examples 10-15:

Western Blot and Pulse Chase Experiments

Cells were lysed in RIPA buffer (50 mM Tris pH 7.4, 150 mM NaCl, 1 mM EDTA, 1% Triton X-100, 1% sodium deoxycholate, 0.1% SDS) and lysates subjected to SDS-PAGE and transferred to Hybond P membrane (Amersham-Pharmacia, Piscataway, N.J.). Blots were revealed by anti-XBP-1 (Santa Cruz Biotechnology, Santa Cruz, Calif.), anti-caspase-12 (J. Yuan, Harvard University, Boston, Mass.), and anti-IRE1α (Urano et al. 2000. Science 287: 664-666) antibodies by standard procedures. HeLa cells were cotransfected with XBP-1u and His-tagged ubiquitin expression plasmids (pMT 107, D. Bohmann, EMBL, Germany) by using the Lipofectamine-2000 reagent (Invitrogen, Carlsbad, Calif.). Cell extracts were purified through Ni-NTA columns as described previously (Campenaro et al. 1997. PNAS 94:2221-2226), and ubiquitinated XBP-1u proteins revealed by western blot analysis with anti-XPB-1 antiserum. Degradation rates of XBP-1u and XBP-1s proteins were determined by pulse labeling J558 cells with ³⁵S Met/Cys for 1 hr and chasing for the indicated times. Radiolabeled XBP-1 proteins were immunoprecipitated from total cell extracts, separated on 10% SDS-PAGE and revealed by autoradiography.

Northern Blot and RT-PCR Analysis

Total RNA was prepared by using Trizol reagent, electrophoresed on 1.2% agarose, 6% formaldehyde gels and then transferred onto Genescreen Plus membrane, (NEN, Boston, Mass.). Hybridizations with ³²P-radiolabeled probes were performed as demonstrated previously (Iwakoshi et al. 2003 Nature Immunology 4: 321-329). Probes for ERdj4 and p58^{IPK} were generated from cDNA excised from EST clones (ATCC, Manassas, Va.) using appropriate restriction enzymes (ERdj4, IMAGE: 1920927; p58^{IPK}, IMAGE:2646147). The ratio of XBP-1u and XBP-1s mRNA was revealed by RT-PCR analysis with a probe set spanning the spliced-out region as demonstrated previously (Iwakoshi et al.2003 Nature Immunology 4: 321-329).

Plasmids and Reporter Assays

Two or three lysine residues in the C-terminus of XBP-1u were replaced by arginines to generate XBP-1uKK (K235R, K252R) and XBP-1uKKK(K146R, K235R, K252R) by site-directed mutagenesis (Iwakoshi et al.2003 Nature Immunology 4: 321-329). dn-XBP contains the N-terminal 188 aa of XBP-1u. NTH3T3 cells were transfected by using the Lipofectamine2000 reagent as recommended by the manufacturer (Invitrogen, Carlsbad, Calif.) with indicated amount of UPRE (UPR element) reporter (Wang et al. 2000 J Biol Chem 275: 27013-27020) and various effector plasmids. Cells were treated for 16 hours before harvest in certain experiments. Cells were lysed in passive lysis buffer for dual luciferase assays according to the manufacturer's protocol (Promega, Madison, Wis.).

Production of iXBP-1 and dn-XBP-1 Myeloma Cells

An XBP-1-specific RNAi vector was constructed by inserting two complementary oligonucleotides for 5′-GGGATTCATGAATGGCCCTTA-3′ (SEQ ID NO: 11) into the pBS/U6 vector as described previously Sui et al. 2002 Proc Natl Acad Sci USA 99: 5515-5520). To make the SGFΔU3 shuttle retroviral vector for RNAi, a polylinker (PmlI, SalI, BamHI and MluI) was inserted between the PmlI and BamHI sites of SFG tcLucECT3 (Lindemann et al. 1997 Mol. Med. 3:466-476). The neomycin resistance gene expression cassette was removed by PCR amplification from the pMCSV vector (Invitrogen) and inserted between the BamHIl and Mlul sites of SGFΔU3 to generate SGFΔU3neo. Lastly, the U6 promoter-iXBP cassette was excised from the pBS/U6-driven vector by SmaI and BamHI digestion and then inserted into SGFΔU3neo between the PmlI and BamHI sites to generate the SGFΔU3neo-iXBP retroviral vector. Retroviral supernatant was prepared and used to transduce J558 cells as described previously (Iwakoshi et al. 2003 Nature Immunology 4: 321-329). Uninfected cells were removed by culturing cells in the presence of 1 mg/ml G418 for more than 1 week. Suppression of XBP-1 mRNA and proteins by RNAi was confirmed by Northern and western blot analysis.

Apoptosis Assays

Cells were stained with annexin V-PE (BD PharMingen, San Jose, Calif.) as recommended and analyzed on a FACScan flow cytometer (Becton Dickinson, San Jose, Calif.).

### Example 10

### Proteasome Inhibitors (PIs) Induce ER Etress but Suppress the UPR in Myeloma Cells

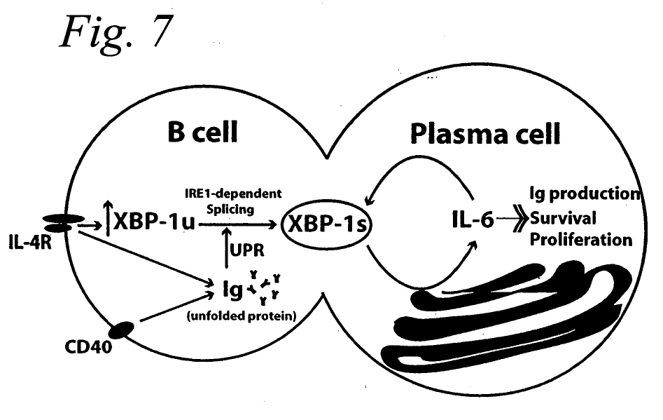

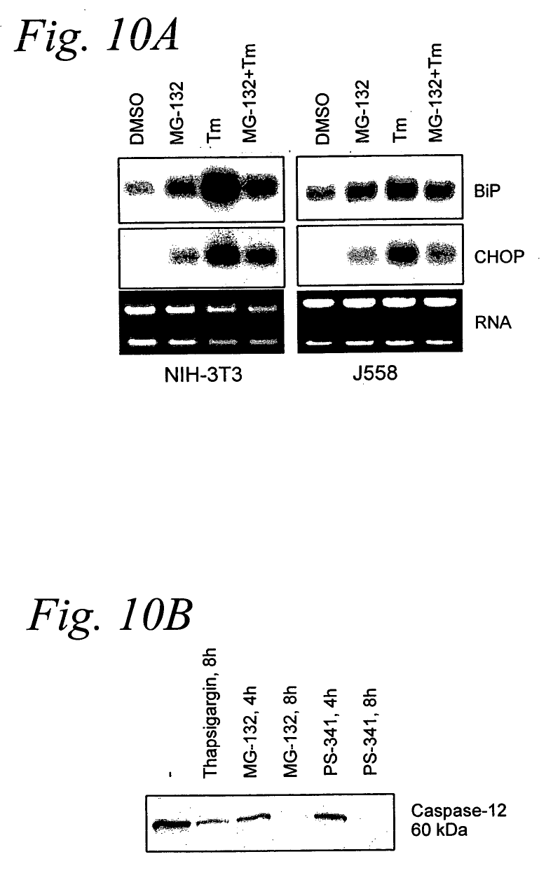

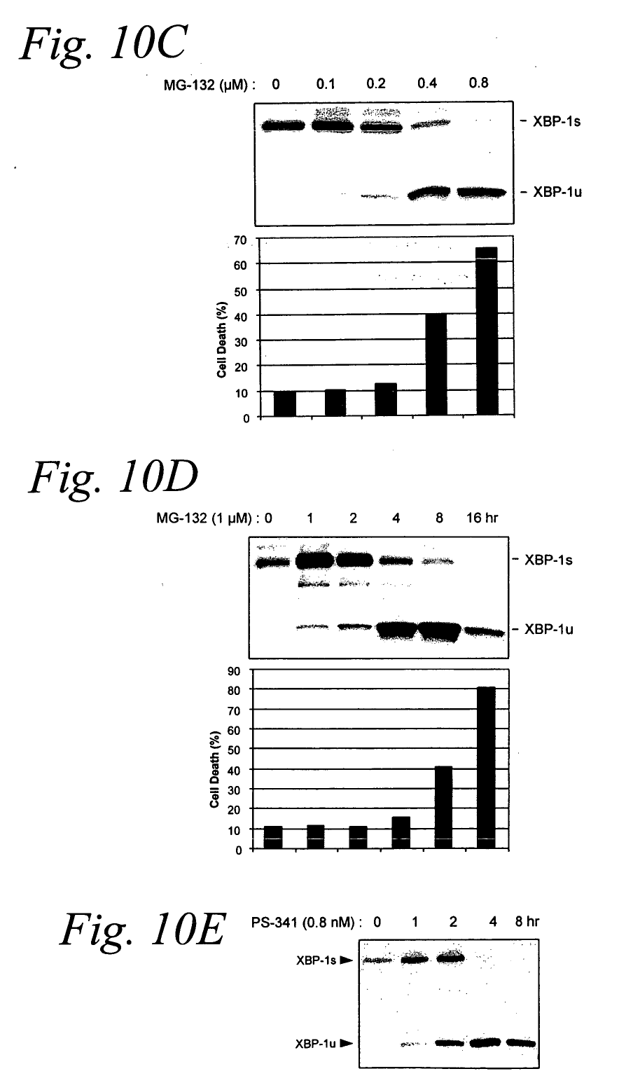

The maturation and folding of ER membrane and secretory proteins relies on the activity of ER-resident chaperones and folding enzymes. ER proteins that ultimately fail to fold properly are degraded by the 26S proteasome, or ERAD. Suppression of proteasome activity induces the accumulation of ERAD substrates in the ER, thereby inducing ER stress. To test the effect of proteasome dysfunction on UPR activation, NIH3T3 fibroblasts (left panel) and J558 myeloma cells (right panel) were treated with the proteasome inhibitor (PI) MG-132 in the presence or absence of the ER stress inducer, tunicamycin (Tm), and the expression of UPR target genes assessed (FIG. 10a). As expected, Tm treatment resulted in the induction of expression of representative UPR target genes such as BiP (Grp78) and CHOP. Treatment of cells with PIs alone also induced the UPR as previously reported (K. T. Bush, et al., J Biol Chem 272, 9086-92. (1997) Y. Kawazoe, et al., Eur J Biochem 255, 356-62 (1998)) (FIG. 10a). PIs also induced caspase 12 activation as evidenced by cleavage of the precursor species (FIG. 7b), confirming that the inhibition of proteasome activity induces ER stress and apoptotic signaling pathways (T. Nakagawa et al., Nature 403, 98-103 (2000)). Surprisingly, however, PI treatment blocked rather than further augmented the Tm-induced stress response in both NIH3T3 and J558 myeloma cells raising the possibility that PIs might also suppress the UPR (FIG. 10a).

### Example 11

### PIs Prevent IRE-1α-Mediated XBP-1 mRNA Splicing

Treatment of J558 cells, which express high levels of the active spliced form of XBP-1 (XBP-1s), with MG132 or PS-341 resulted in a striking accumulation of XBP-1 u and a concomitant decrease in XBP-1s proteins at concentrations of MG-132 between 0.2 and 0.4 μM (FIG. 10c). A time course of the kinetics of induction and loss of the two XBP-1 species by MG-132 revealed that XBP-1s was induced at early time points, but rapidly declined after 4 hours of treatment and was barely detectable by 16 hours (FIG. 10d). Conversely, XBP-1u levels were increased from as early as one hour post treatment and were sustained throughout the experiment, peaking at 8 hours (FIG. 10d). Of note, MG-132 and PS-341 induced apoptosis in J558 cells (FIG. 10c, d). A close correlation between the dose dependency of the XBP-1s to unspliced shift and apoptosis was observed, with the most marked increase in both occurring between 0.2 and 0.4 μM MG-132 (FIG. 10c). Similarly, the kinetics of XBP-1u accumulation and XBP-1s loss mirrored the kinetics of MG-132-induced apoptosis of these cells (FIG. 10d). PS-341 induced the same marked shift in the ratio of XBP-1s to XBP-1u in the human MM cell line MM.1s (FIG. 10e) and in primary MM cells derived from patient bone marrow.

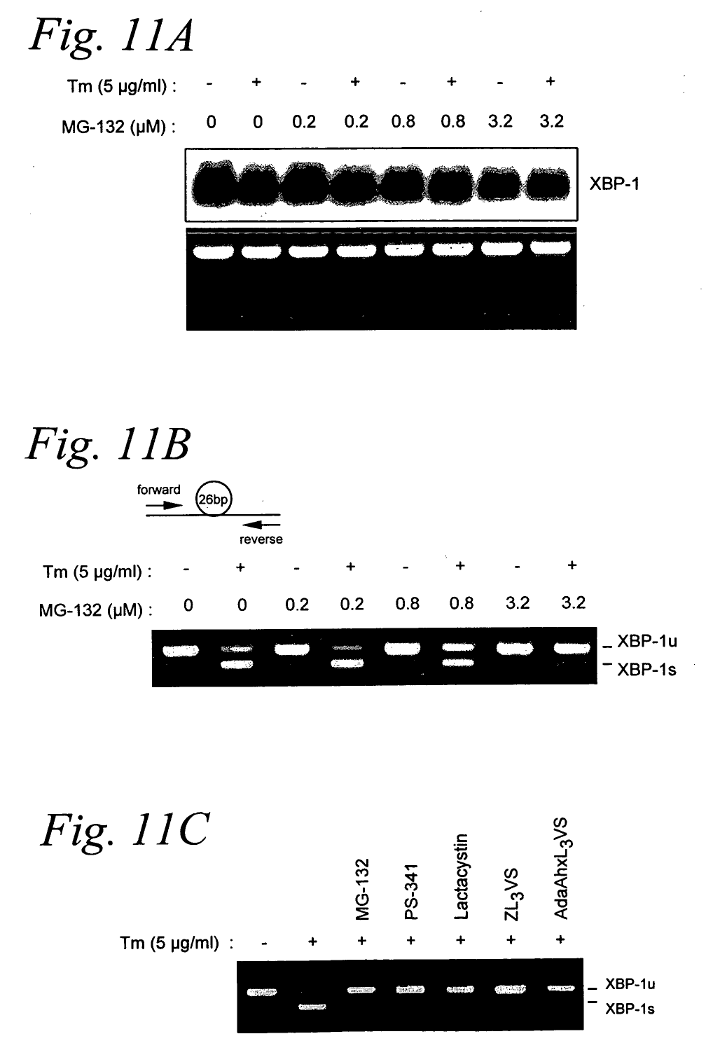

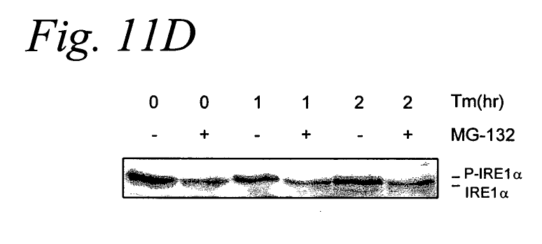

The disappearance of the spliced XBP-1s species suggested that PIs suppressed IRE1α-mediated XBP-1mRNA splicing. Overall levels of XBP-1 RNA were not significantly altered by either Tm or MG-132 in J558 cells by Northern blot analysis which does not distinguish between XBP-1u and s transcripts (FIG. 11a). Relative amounts of XBP-1u and s transcripts were measured by RT-PCR with a primer set that amplified 145 bp and 119 bp of XBP-1u and XBP-1s mRNA, respectively (FIG. 11b). As expected, Tm treatment markedly induced XBP-1 mRNA splicing (FIG. 11b, first two lanes). MG-132 alone did not induce any XBP-1mRNA splicing even after prolonged treatment up to 8 hours at high concentrations (FIG. 11b). Interestingly, however, Tm-induced XBP-1 mRNA splicing was suppressed by MG-132 in a dose dependent manner as reflected by a decrease of the ratio of XBP-1s to unspliced forms (FIG. 11b). The marked decrease in XBP-1s protein following MG-132 treatment results from suppression of IRE1-mediated XBP-1 mRNA splicing. To confirm that MG-132 inhibited XBP-1 splicing by targeting the proteasome, a panel of compounds known to specifically inhibit proteasomal activity was tested. PS-341, a reversible inhibitor of chymotryptic activity of the 20S proteasome complex, ZL₃VS and AdaAhx₃L₃VS, both of which efficiently target all β subunits of the proteasome (B. M. Kessler et al., Chem Biol 8, 913-29 (2001)), all suppressed XBP-1 mRNA splicing as efficiently as MG-132 (FIG. 11c), confirming that MG-132 inhibited XBP-1 splicing by targeting the proteasome.

Upon sensing misfolded proteins in the ER lumen, IRE1 proteins become activated by oligomerization and autophosphorylation. To determine at what step PIs interfered with IRE1α function, the integrity of IRE1α phosphorylation was assessed. Western blot analysis of extracts prepared from untreated and Tm-treated cells revealed the increase in the phosphorylated (slower mobility) and decrease in unphosphorylated IRE1α species previously observed (FIG. 11d). Notably, MG-132 completely blocked the phosphorylation of IRE1α by Tm (FIG. 11d). These data demonstrate that the initial steps of IRE1α activation are disrupted in the presence of PIs, resulting in impaired oligomerization and autophosphorylation.

### Example 12

### The Stabilized XBP-1u Protein Acts as an Inhibitor of the Spliced Species

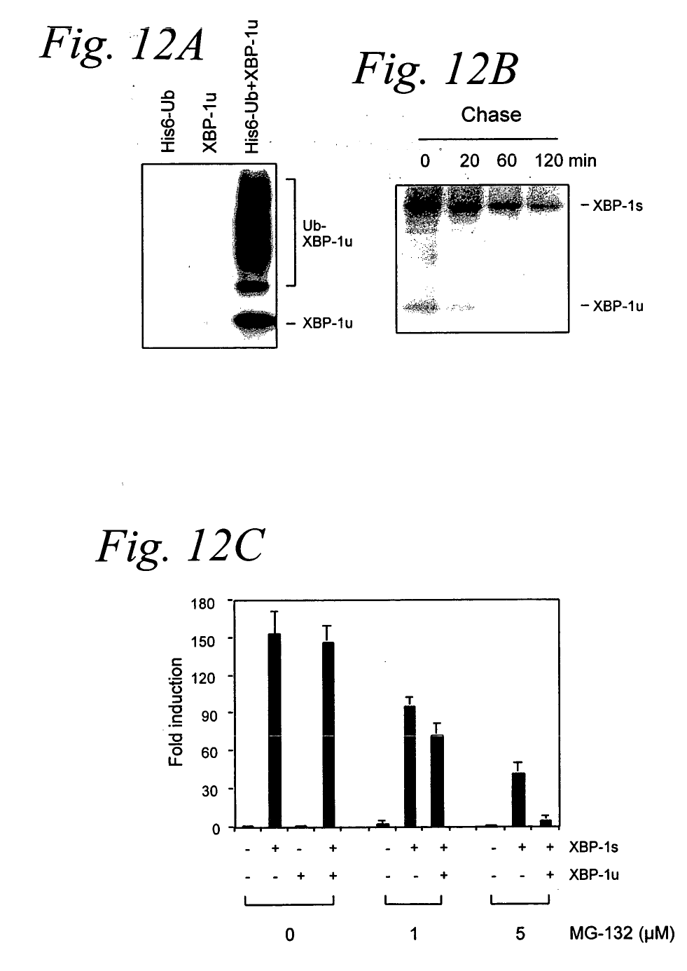

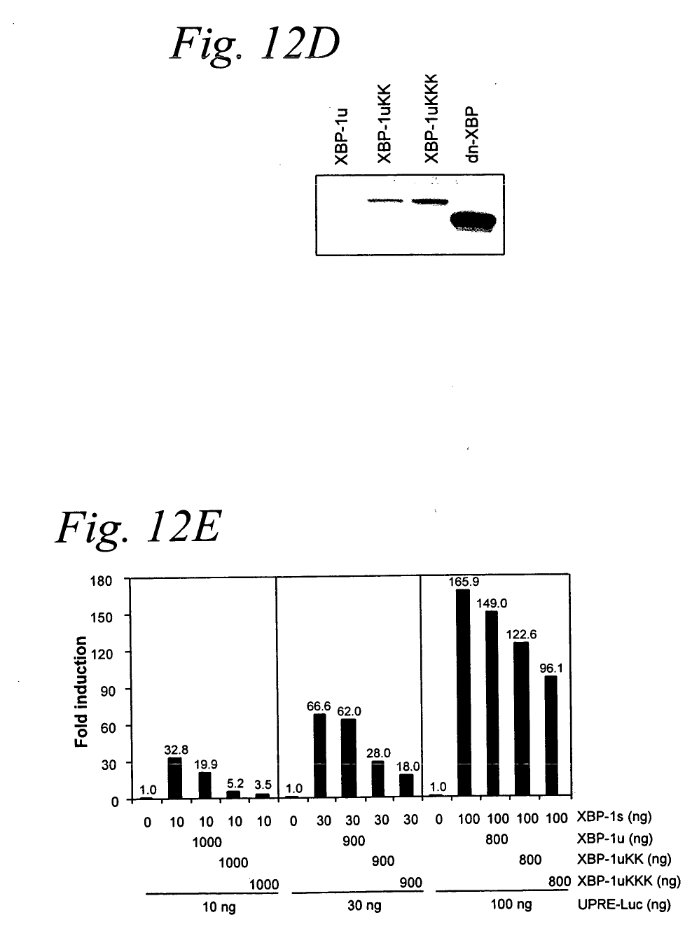

PIs could act at multiple stages to alter the balance between XBP-1 unspliced and spliced species. Two potential mechanisms were suppression of the splicing event itself, or preferential stabilization of XBP-1u protein. Under normal conditions, XBP-1u protein is barely detectable in J558 cells despite the presence of abundant XBP-1u transcripts, indicating its poor stability (H. Yoshida, T. Matsui, A. Yamamoto, T. Okada, K. Mori, Cell 107, 881-891(2001)). Indeed, XBP-1u protein is highly ubiquitinated in vivo (FIG. 12a) and rapidly degraded in myeloma cells with a half-life of approximately ten minutes (FIG. 12b). Thus XBP-1u protein is rapidly degraded through the ubiquitin-proteasome pathway, and is stabilized and accumulates in the presence of PIs. XBP-1s protein, while also unstable, has a longer half life of approximately one hour. In conclusion, the initial accumulation of XBP-1s protein at early time points and the rapid increase in XBP-1 uprotein after MG-132 treatment reflect their stabilization by PIs while the rapid decline of XBP-1s protein level thereafter is explained by suppression of IRE1α-dependent XBP-1 splicing.

The XBP-1s, but not XBP-1u protein possesses a potent transactivation domain (H. Yoshida, T. Matsui, A. Yamamoto, T. Okada, K. Mori, Cell 107, 881-891 (2001)) and reconstitutes Ig secretion in B cells N. N. Iwakoshi et al., Nature Immunology 4, 321-329 (2003). Since XBP-1u shares the leucine zipper motif at the N-terminus, it was possible that it might partner with XBP-1s to regulate its activity. To test this, NIH3T3 cells were cotransfected with an XBP-1s expression plasmid in the presence or absence of XBP-1u and a UPRE-luciferase reporter plasmid. In the absence of treatment, XBP-1s but not XBP-1u, greatly increased reporter activity (FIG. 12c). In the presence of MG-132, however, XBP-1u now significantly suppressed transactivation of the reporter by XBP-1s, suggesting that the accumulated, stabilized XBP-1u protein acted as a dominant negative to suppress the activity of the spliced species (FIG. 12c).

To more directly investigate whether XBP-1u protein acted as a dominant negative inhibitor of XBP-1s, it was necessary to avoid other potentially complicating actions of the PIs. More stable forms of XBP-1 unspliced were produced by changing lysine residues in the C-terminus, the site of potential XBP-1u ubiquitination, to arginine. XBP-1uKK and XBP-1uKKK, mutant proteins in which two and three C-terminal lysines, respectively, have been replaced with arginine, are expressed at a higher level than the original XBP-1u protein consistent with a role for ubiquitination-dependent degradation (FIG. 12d). These more stable mutant forms of XBP-1u inhibited the transactivation of the reporter by XBP-1s even in the absence of PIs (FIG. 12e). Thus, the unspliced version of XBP-1u can act as a dominant negative inhibitor of the spliced form when its expression is stabilized by interference with its degradation by ubiquitination, a situation that occurs in myeloma cells in the presence of PIs.

### Example 13

### Absence of Functional XBP-1 Increases ER Stress-Induced Apoptosis of Myeloma Cells

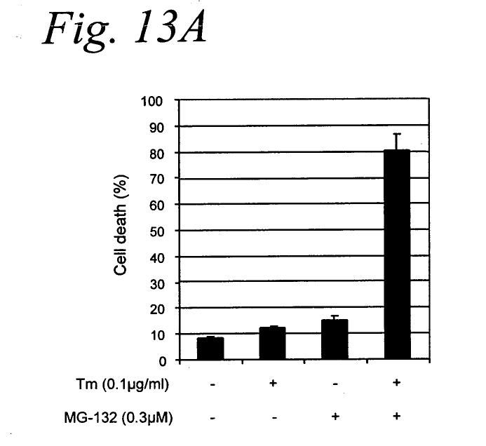

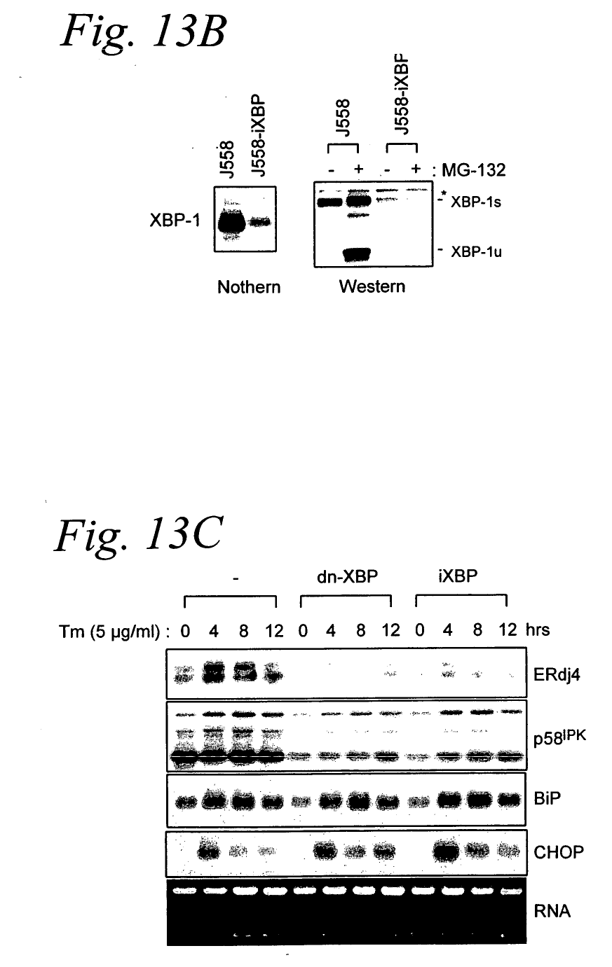

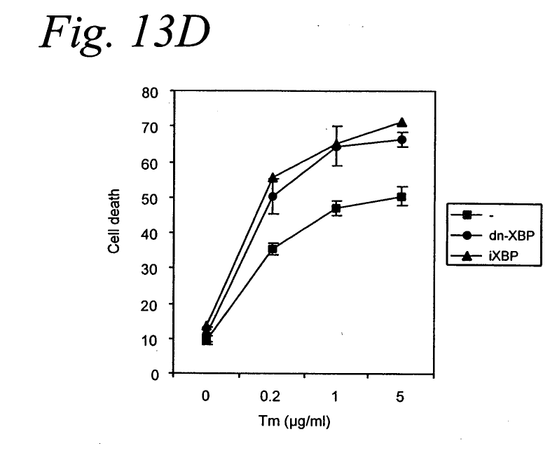

While PIs heighten ER stress in myeloma cells by preventing the degradation of ERAD substrates, they paradoxically inhibit UPR activation. It was possible that PIs would induce apoptosis in part by inducing ER stress and subsequent apoptotic signaling pathways while simultaneously preventing an appropriate UPR. Consistent with this hypothesis, Tm and MG-132 synergistically induced apoptosis in J558 myeloma cells (FIG. 13a). These results indicate that Tm and MG-132 augmented ER stress by increasing the input of misfolded proteins and blocking ERAD degradation, respectively.

To further test the effect of an impaired UPR on the handling of ER stress, myeloma cell lines functionally deficient in XBP-1 were generated either by transducing J558 cells with a potent dominant negative XBP-1 retrovirus (dn-XBP-1) or with an siRNA retrovirus (iXBP). Since dnXBP-1 does not possess the C-terminal destabilization motif present in XBP-1u, it is expressed at high levels (FIG. 12d, lane 4) and inhibits XBP-1s -induced transactivation very efficiently. Suppression of XBP-1 expression in the iXBP-transduced cells was confirmed by both Northern and Western blot analysis (FIG. 13b). The functional impairment of XBP-1 activity was demonstrated by the greatly reduced induction of XBP-1-dependent UPR target genes, ERdj4 and p58^{IPK} (J. Kurisu et al., Genes Cells 8, 189-202 (2003);Y. Shen, et al., J Biol Chem 277, 15947-56 (2003)) by Tm in both the dn-XBP-1 and iXBP-1 transduced cells (FIG. 13c). In contrast, the induction of BiP and CHOP, which are not regulated by XBP-1, was minimally affected by dn-XBP or XBP-1 RNAi. No effect on cell proliferation or viability was observed at baseline. However, upon Tm treatment, dnXBP-1 and iXBP-1 myeloma cells both displayed significantly increased apoptosis when compared to control GFP-transduced J558 cells (FIG. 13d), suggesting that the IRE1α/XBP-1 pathway contributes to the survival of myeloma cells under ER stress conditions.

These data indicate that a functional UPR is necessary to protect XBP-1^{-/-} cells from stress-induced death. Without wishing to be bound by theory, PIs may cause apoptosis of myeloma cells, the malignant counterpart of the plasma cell, by interfering with the UPR. While the partial but not complete protection from apoptosis observed in the functionally XBP-1 deficient myeloma cells may be partly attributed to residual XBP-1 activity, PIs affect other cellular pathways that impinge on apoptosis (Y. Yang, et al., Science 288, 874-7 (2000)).

Two additional UPR signaling pathways involve the activation of transcription factor ATF6 or translational repression mediated by PERK/eIF2α. ATF6, like XBP-1α basic region/leucine zipper transcription factor, is a second ER transmembrane component which is constitutively expressed in an inactive form until ER stress results in proteolytic cleavage of its N-terminal cytoplasmic domain by the S2P serine protease to produce a potent transcriptional activator of chaperone genes (H. Yoshida, et al., Cell 107, 881-891 (2001);, H. Yoshida, et al. J. Biol. Chem. 273, 33741-33749 (1998); J. Shen,et al., Dev Cell 3, 99-111 (2002) J. Ye et al., Mol Cell 6, 1355-64 (2000); M. Li et al., Mol Cell Biol 20, 5096-106 (2000) Y. Wang et al., J Biol Chem 275, 27013-20. (2000). dn-XBP-1 potently inhibited the function of ATF-6 through heterodimerization. Cell death in dnXBP-1 transduced myeloma cells did not, however, exceed that observed in the iXBP-1 J558 cell line (FIG. 13d), as would have been expected if both factors were significant targets for PIs.

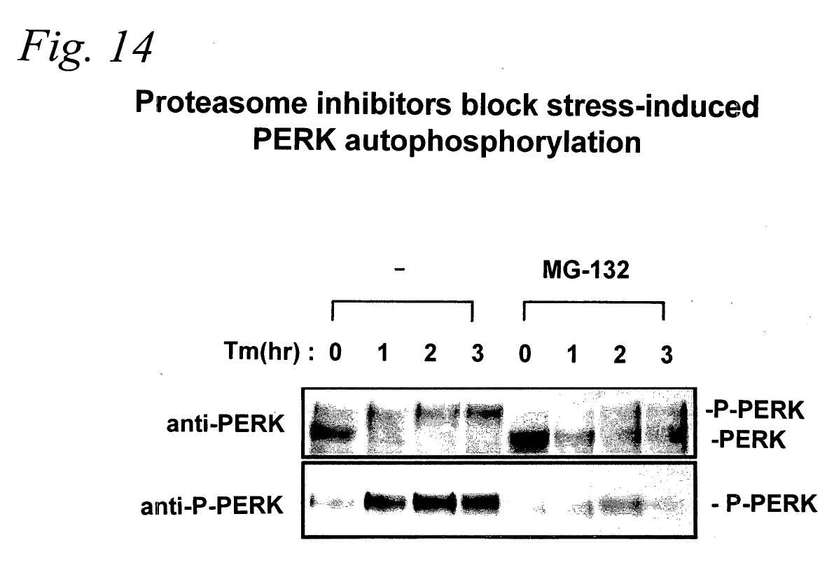

A third ER transmembrane component, PEK/PERK, like IRE1α, is a type 1 transmembrane serine/threonine protein kinase that undergoes ER-stress-induced dimerization of its lumenal domain, autophosphorylates and then acts in the cytoplasm to phosphorylate eIF2α. Phosphorylation of eIF2α leads to translation attenuation in response to ER stress (H. P. Harding, Y. Zhang, D. Ron, Nature 397, 271-274 (1999); Y. Shi et al., Mol. Cell. Biol. 18, 7499-7509 (1998)). The induction of the stress response gene CHOP, shown to be PERK dependent (F. Urano, A. Bertolotti, D. Ron, J. Cell Sci. 113, 3697-3702 (2000)), is prevented by PIs (FIG. 10a) suggesting that PIs might also target PERK. Further, since the ER luminal domain of IRE1 and PERK are interchangeable and conserved throughout evolution, the mechanism by which PIs inhibit IRE1α and PERK activation will be similar. As expected, treatment of J558 cells with Tm led to an increase in the amount of phosphorylated PERK as assessed using an anti-PERK antibody (a shift upwards in mobility of the PERK species) and, more conclusively, using an antibody that recognizes only phospho-PERK. Notably, inclusion of MG-132 resulted in a very marked decrease in the autophosphorylation of PERK, similar to what we had observed for IRE1α (FIG. 14). Little is known about the factors that control activation of IRE1α. To date, BiP and TRAF2 are the only proteins reported to interact with IRE1α (F. Urano et al., Science 287, 664-666 (2000); A. Bertolotti, Y. Zhang, L. M. Hendershot, H. P. Harding, D. Ron, Nat. Cell Biol. 2, 326-332 (2000)), and the mechanism by which PIs alter the activity of the endoribonuclease function of IRE1α is also not known. The data show that PIs modestly induce UPR target genes, consistent with previous reports (Bush et al. 1997. J. Biol. Chem 272:9086; Kawazoe et al. 1998. Eur. J. Biochem 255:356). However, PIs inhibit the stress-induced UPR as evidenced by suppression of IRE-1 mediated XBP-1 mRNA splicing and stabilization of XBP-1 unspliced protein as well as PERK autophosphorylation. Similarly, MG-132, but not Tm treatment of XBP-1-deficient MEFs (mouse embryo figroblasts), resulted in the normal induction of ERdj4 (an XBP-1-dependent UPR target gene) suggesting that MG-132 and Tm induce UPR target genes through distinct mechanisms. Without wishing to be bound by theory, PIs may induce distinct transcription factors (e.g., heat shock factors), which in turn induce both cytosolic Hsps as well as ER-resident chaperones. The IRE1α/XBP-1 pathway contributes to the survival of myeloma cells under ER stress conditions.

Most data suggest that proteasome inhibition induces cell death in proliferating cells while it inhibits apoptosis in differentiated cells such as thymocytes and sympathetic neurons. Thus, PIs induced apoptosis in human glioma cells, human T-cell leukemia cells and PC-12 cells while estopside-induced apoptosis in thymocytes was suppressed with peptide aldehyde PIs (Wagenknecht, B. et al. 2000. J. Neurochem 75:2288; Kitagawa, H. et al. 1999. FEBS Lett 443:181; Stefanelli, C. 1998. Biochem J. 332:661). Apoptosis in glimoa cells is morphologically characterized by dialted rough ER, cytoplasmic vacuoles and dense mitochondrial deposits. Interestingly, this histologic picture was not affected by the broad caspase inhibitor zVADfmk although apoptosis was inhibited. Another study demonstrated that PI-induced glimoa cell death was associated with mitochondria-independent caspase-3 activation. The instant examples show that PIs induce apoptosis of myeloma cells by the novel mechanism of disrupting the UPR. Blockade of the IRE1α/XBP-1 pathway by PIs contributes to the death of myeloma cells under ER stress conditions. The mechanisms by which PIs induce apoptosis may depend upon the status of differentiation, proliferation, activation, or function of a given cell. Secretory cells that require an active UPR and ERAD to ensure proper processing of proteins in the ER, can be particularly susceptible to apoptosis by agents that evoke ER stress but disrupt the UPR. These data show that compounds that inhibit the UPR by targeting the activity of IRE1/XBP-1, alone or in combination with known anti-cancer therapies or agents that induce ER stress and/or disrupt the UPR (e.g., proteasome inhibitors) will be potent therapeutic agents, e.g., for the treatment of disorders such as multiple myeloma and other tumors (for example, adenocarcinomas of the prostate, breast and ovary, that originate from secretory cells).

### Example 14

### Generation of a Potent XBP-1 Dominant Negative Protein

Versions of XBP-1 with various deletions of the C terminal transcription activating domain (e.g., consisting of the N terminal 225, 188, or 136 amino acids of the spliced form of XBP-1) that expressed high levels of protein by western analysis were created. The proteins were tested for their ability to inhibit the transactivation function of spliced XBP-1. Both the 188 and 136 N terminal mutants were extremely potent inhibitors (effective at a 1:1 ratio) of the spliced XBP-1. NIH 3T3 cells expressing XBP-1uc constructs were transfected with 200 ng of spliced XBP-1, yielding a relative luciferase activity of 44.5. 200 ng of XBP-N188 lowered the luciferase level to 1.8 and 200 ng of XBP-N136 lowered the luciferase activity to 2.2.

### Example 15

### Identification of Genes Regulated by XBP-1

DNA microarray analysis was used identify genes regulated by XBP-1. Gene expression in MEFs derived from XBP-1 deficient embryos was compared to that in wildtype MEFs. Gene expression both in the absence of and in the presence of tunicamycin, an agent which evokes the UPR, was analyzed. Analysis yielded several differentially expressed genes, one of which was DNAJB9, encoding mDj7. MDj7 is a small type II protein of 222 amino acids. The expression of mDj7 was induced by treatment of wildtype cells, both MEFs and B cells, with tunicamycin and LPS respectively, but was absent in XBP-1 null MEFs and B cells. The DNAJB9 promoter is induced by XBP-1; the function of XBP-1 in regulating mDj7 is accounted for by direct transactivation of the mDj7 promoter by XBP-1. Further, using the tetracycline-regulated off system of inducible gene expression with constructs encoding spliced and dominant negative XBP-1, it was shown that the expression of mDj 7 is regulated by and absolutely dependent on XBP-1. This is in contrast to members of the DnaK/Hsp70 family of genes such as BiP/Grp78 and CHOP10, whose expression is XBP-1 independent. These data allow for the classification of subsets of chaperone genes as defined by dependence on XBP-1.

The following Materials and Methods were used in Examples 16-22

Cell Culture and Cell Lines

293T and MEF cells derived from wild type and XBP-1^{-/-} embryos were cultured in DMEM supplemented with 10% fetal calf serum (Hyclone Laboratories). MEF-tet-off cells (Clontech) were maintained in the same media with the addition of 100 μg/ml G418 and 1 μg/ml doxycycline. XBP-1s inducible cells were obtained by transfecting MEF-tet-off cells with the TREhyg-XBP-1s plasmid and then selecting in the presence of 400 μg/ml hygromycin B. Several clones were tested for doxycycline-dependent XBP-1s expression, and one was selected for further experiments. MEF-dn-XBP cells were generated by transfecting MEF-tet-off cells with the TREhyg-dn-XBP plasmid. Because dn-XBP did not affect cell viability and growth, MEF-dn-XBP cells were maintained in media without doxycycline. ATF6α and β knockdown MEF cells were generated by cotransfecting wild type MEF cells with U6-iATF6α and β and cmv-puromycin or transducing cells with retroviruses containing each RNAi vector.

GeneChip Analysis

Total RNA was isolated from MEF cells with TRIZOL reagent (Invitrogen, Carlsbad, Calif.). cDNA synthesis, hybridization and laser scanning of the array were carried out at the Gene Array Technology Center (Brigham and Women's Hospital, Boston, Mass.) with MG-U74A GeneChips which had 6,000 functionally characterized sequences and 6,000 ESTs from the UniGene database (Affymetrix, Santa Clara, Calif.) as recommended by Affymetrix. Data analysis was performed using Affymetrix GeneChip 3.1 software under default parameter setting.

Northern Blot Analysis

Total RNA was prepared by using Trizol reagent, electrophoresed on 1.2% agarose, 6% formaldehyde gels and then transferred onto Genescreen Plus membrane (NEN). ³²P-radiolabeled probes were prepared with the RediPrime II labeling system (Amersham-Pharmacia). Template DNAs for the probes were cut out by using appropriate restriction enzymes from the cDNA-containing plasmid (XBP-1, 15-830 of the murine coding region) or EST clones from ATCC (CHOP, IMAGE:5863055; ERdj4, IMAGE: 1920927; p58^{IPK} probe A, IMAGE:9001935; p58^{IPK} probe B, IMAGE:2646147; ATF6α, IMAGE:4503659; MGP, IMAGE:4990627; EDEM, IMAGE:5324660; PDI-P5, IMAGE:2645183; RAMP4, IMAGE:3489738). Grp94 and Grp78 probes were kindly provided by R. J. Kaufman, Univ. of Michigan and the HEDJ probe the kind gift of L. Hendershot, St. Jude Children's Research Hospital, Memphis. Probe hybridization was performed with Ultrahyb buffer as recommended by the manufacturer (Ambion).

Plasmid Construction and Transient Transfection Assays

To make 4×XBPGL3, two complementary oligonucleotides containing the XBP-1 binding site 5^{f}-CGCG(TGGATGACGTGTACA)₄-3′ (SEQ ID NO: 12) and 5′-GATC(TGTACACGTCATCCA)₄-3^{f} (SEQ ID NO: 13) were annealed and ligated to the—40-Luc plasmid (Lee et al. 2000. Biochem J. 350 Pt1:131-138) digested by Mlu I and Bgl II. The UPRE reporter was constructed by inserting an annealed oligonucleotide containing two UPRE motifs (Roy and Lee 1999 Nucleic Acids Res. 27:1437), 5′-cgcgtcaCCAATcggaggcctCCACGaccaCCAATcggaggcctCCACGac-3′, (SEQ ID NO: 14) to the 40-Luc plasmid between the Mlu I and Xho I sites (Lee et al. 2000. Biochem J. 350 Pt1:131-138). UPRE reporter (5×ATF6GL3), pCGNATF6 and pCGNAF6 (1-373) were previously described (Wang et al. 2000. J. Biol. Chem. 275:27013). The pCDNA3.1 (Clontech) driven expression vectors for mouse XBP-1s and XBP-1u/s were described elsewhere (Iwakoshi et al. 2003. Nature Immunology 4:321). pCDNA-dn-XBP was constructed by removing the region downstream of the Eco RV site of XBP-1s cDNA in the pCDNA-XBP-1s plasmid. TREhyg-XBP-1s and TREhyg-dn-XBP were constructed by inserting the XBP-1s cDNA and DN-XBP, respectively, into TRE2 hyg (Clontech) between the Pvu II and Sal I sites. 0.5 kb fragment of the ERdj4 promoter was PCR amplified from a C57BL6 mouse genomic DNA with the following primer set; 5′ AGGCTTGGGCTCTAATGGCCTCTCAA-3′ (SEQ ID NO: 15) and 5′-CTCCGAACGCCGAGTAGCCT-3′ (SEQ ID NO: 16), and then inserted into the pGL3-basic (Promega) plasmid between Nhe I and Xho I to generate ERdj4GL3. MEF cells were transfected by using the Lipofectamine2000 reagent as recommended by the manufacturer (Invitrogen). Briefly, 1 μg of DNA and 3 μl of Lipofectamine 2000 reagent were diluted each in 100 μl OPTI-MEM, mixed and added to cells in 12 well plates at 60,000 cells per well. Six hours later, cells were washed and cultured for 16 hours in fresh media with or without 1 μg/ml Tm. For dual luciferase assays, 50 or 100 ng reporter and 10 ng of RL/cmv (Promega) plasmids were cotransfected with various amounts of effector plasmids and pCDNA3.1 which was added to give 1 μg of DNA in total. Cells were lysed in passive lysis buffer for dual luciferase assays according to the manufacturer's protocol (Promega). 293T cells plated in a 10 cm dish were transfected with 5 μg of each expression plasmid by the standard calcium phosphate method.

Immunoprecipitation and Western Blot Analysis

48 hours after transfection, 293T cells were washed twice with cold PBS and lysed in 1 ml lysis buffer (10 mM Tris pH7.4, 150 mM NaCl, 1% Triton X-100, 1 mM EDTA) containing a protease inhibitor cocktail tablet (Roche). Lysates were precleared by using protein A-agarose beads (Roche) for 1 hour and incubated with agarose conjugated anti-HA antibody (Santa Cruz) for overnight. The agarose beads were washed five times with the lysis buffer and resuspended in SDS-PAGE sample buffer. MEF cells were lysed in RIPA buffer (50 mM Tris pH 7.4, 150 mM NaCl, 1 mM EDTA, 1% Triton X-100, 1% sodium deoxycholate, 0.1% SDS). Lysates and immunoprecipitates were subjected to SDS-PAGE and transferred to Hybond P membrane (Amersham-Pharmacia). Blots were revealed by anti-XBP-1 (Santa Cruz), anti-ATF6β or anti-ATF6α (kind gift from Dr. K. Mori, Kyoto University, Japan) antibodies by standard procedures.

Knock-Down of ATF6α and ATF6β

The pBS/U6 plasmid was used (Sui et al. 2002. Proc. Natl. Acad Sci USA 99:5515). The two complementary oligonucleotides were annealed and inserted into pBS/U6 between blunt-ended Apa I and Eco RI sites to generate U6-iATF6oc; 5′-GGCAGTGTCGCCTGGTGTTGaagcttCAACACCAGGCGACACTGCCCtttttg-3′ (SEQ ID NO: 17)5′-aattcaaaaaGGGCAGTGTCGCCTGGTGTTGaagcttCAACACCAGGCGACACTGCC-3′ (SEQ ID NO: 18). Similarly, an ATF6β,-specific RNAi vector was constructed by inserting two complementary oligonucleotides for 5′-GGGTGGCAGAAGTCAGTTTATG -3′(SEQ ID NO: 19) into the pBS/U6 vector. To make the SGFΔU3 shuttle retroviral vector for RNAi, a polylinker (Pmll, Sail, BamHI and Mlul) was inserted between the Pmll and BamHI sites of SFG tcLucECT3 (Lindemann et al. 1997. Mol. Med 3:466). The hygromycin and puromycin resistance gene expression cassettes were removed by PCR amplification from the pMCSV series vectors (Invitrogen) and inserted between the BamHI and Mlul sites of SGFΔU3 to generate SGFΔU3 hyg and SGFΔU3pur, respectively. Lastly, the U6 promoter-iATF cassettes were excised from the pBS/U6-driven vectors by Smal and BamHI digestion and then inserted into SGFΔU3 hyg or SGFΔU3pur between the Pmll and BamHI sites to generate the retroviral vectors for iATF6α or iATF6β with various drug selection markers. Retroviral supernatant was prepared and used to transduce MEF cells as described previously (Iwakoshi et al. 2003). Uninfected cells were removed by culturing cells in the presence of 200 μg/ml hygromycin B or 2 μg/ml puromycin for more than 1 week.

### Example 16

### Identification of Known and Novel UPR Genes by DNA Mier Array Analysis

A genome-wide analysis in yeast revealed that a subset of genes including ER-resident chaperones, and those involved in phospholipid biosynthesis and protein degradation pathways were induced through the UPR (Travers, K. J., et al. 2000. Cell 101: 249-258). However, very few UPR target genes are known in mammalian cells. To identify mammalian UPR target genes that were differentially regulated by XBP-1, ATF6α and ATF6β oligonucleotide based gene array analysis was used on RNAs from MEF cells untreated or treated with tunicamycin (Tm) for 6 hours. Expression of 2.8% of the total pool of 12,000 genes analyzed was increased upon Tm treatment. To avoid the detection of false positive genes, Tm-inducible genes were sorted by ratio of oligonucleotide probe pairs whose values were increased upon Tm treatment, and the genes whose values were >0.8 are shown in Table 1. As expected, a few, well-known UPR target genes including CHOP, GADD45, Herp, BiP, and XBP-1 were significantly induced by Tm treatment with fold inductions ranging from 4 to 27. Interestingly, Tm treatment induced the expressi on of several transcription factors including CHOP, LRG-21, XBP-1, NF-IL3/E4BP4 and ATF-4, which have leucine zipper motifs. Given the known ability of transcription factors containing the leucine zipper motif to homo and heterodimerize it will be of interest to test for possible interactions among these proteins.

### Example 17

### Induction of the ERdj4 and p58^{ipk}DnaJ/Hsp40-Like Accessory Genes upon ER Stress Requires XBP-1

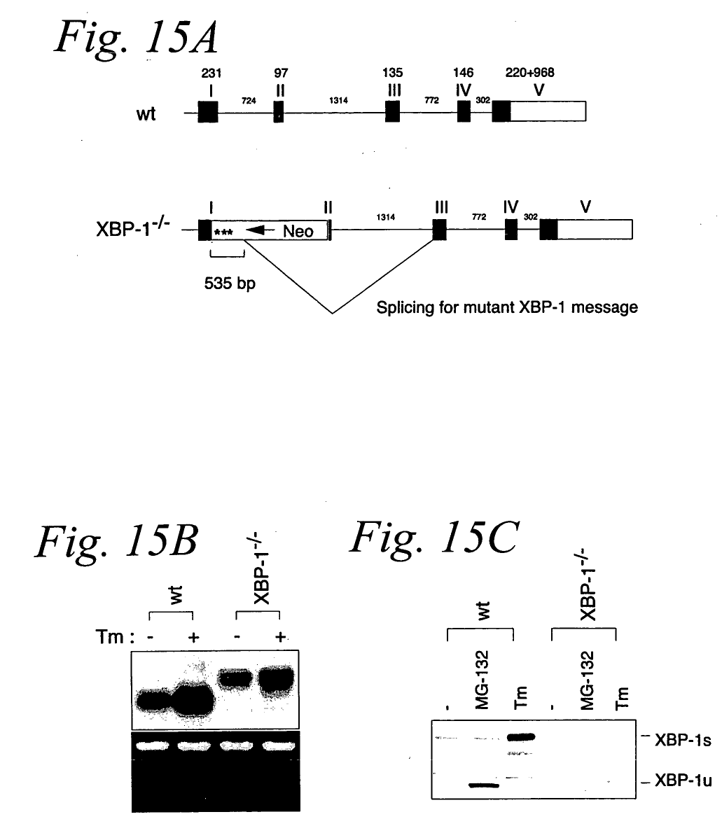

To investigate the requirement of XBP-1 in UPR target gene expression, MEF cells were generated from XBP-1 deficient mice (Reimold, A. M., et al. 2000. Genes Dev. 14: 152-157). Treatment of wt MEFs with the proteasome inhibitor MG-132 or with Tm induced both XBP-1u and XBP-1s proteins (FIG. 15B) through protein stabilization and mRNA splicing, respectively, as demonstrated previously (Yoshida, H., et al. 2001a. Cell 107: 881-891). In contrast, as expected, neither XBP-1 protein species was produced in XBP-1^{-/-} MEF cells, because of multiple stop codons derived from the neo cassette (FIGS. 11A, C). XBP-1-dependent UPR target gene expression was searched for using gene array analysis with the RNA from the XBP-1 deficient MEF cells untreated or treated with Tm. It has been previously shown that the expression of neither BiP nor CHOP, the prototypical ER stress chaperone genes, was affected by loss of IRE1α, raising the possibility that these chaperone genes were regulated by other UPR pathways (Urano, F., Bertolotti, A., and Ron, D. 2000a. J. Cell Sci. 113: 3697-3702; Lee, K., et al., 2002. Genes Dev. 16: 452-466.). Not surprisingly, therefore, most of the prototypical UPR target genes identified in wild type MEF cells were normally induced in XBP-1^{-/-}0 MEF cells, invoking the presence of additional UPR signaling pathways that are not dependent on XBP-1 (Table 1).

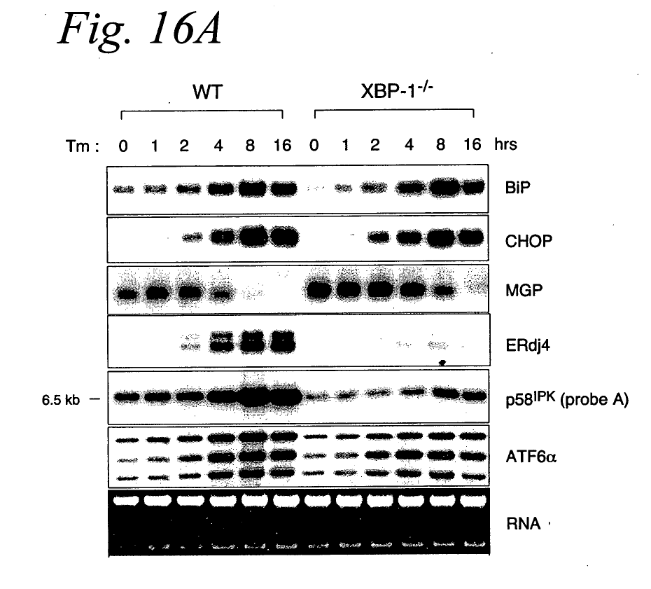

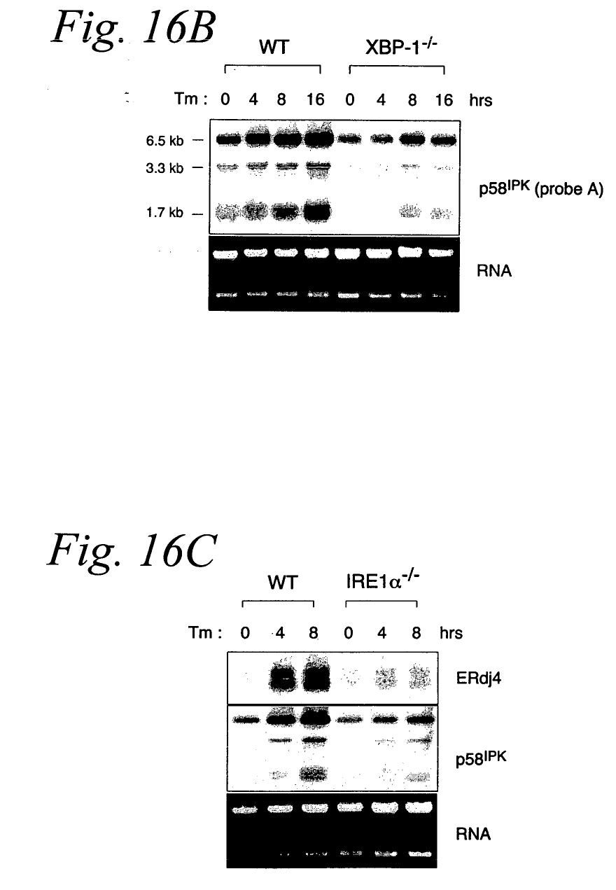

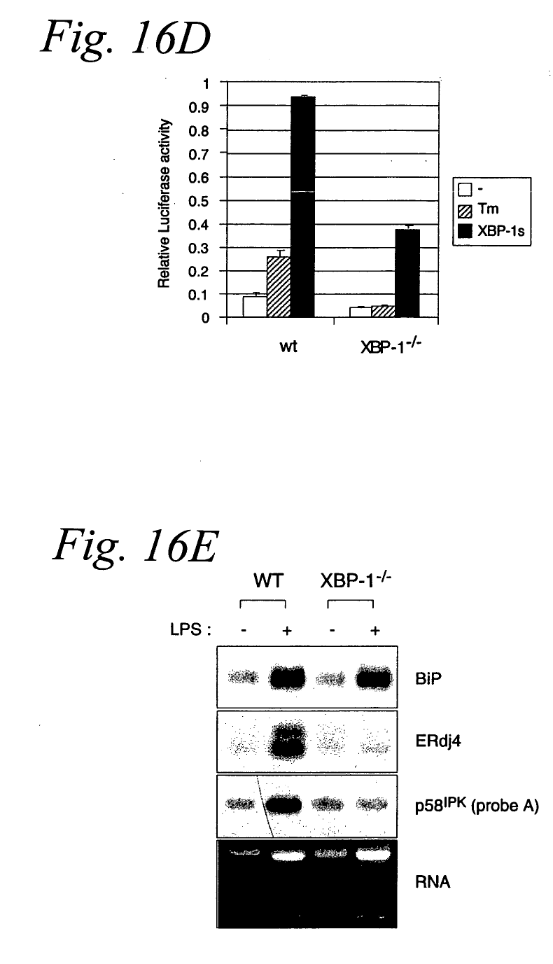

In contrast, several known and novel UPR target genes were identified which failed to be induced by Tm in the XBP-1 null MEFs. To minimize gene array artifacts, RNA levels were compared between Tm-treated wild type and XBP-1^{-/-} MEF cells. Two UPR target genes, ERdj4 and p58^{ipk}, were not induced at all in XBP-1^{-/-} MEF cells by this analysis, a finding which was verified by Northern blot analysis (FIG. 16A). In time course experiments, BiP and CHOP expression were only modestly impaired in the absence of XBP-1 (FIG. 16A).

Gene array analysis suggested that MGP mRNA, an inhibitor of calcification of arteries and cartilage (Luo et al. 1997. Nature 386:78), was Tm-inducible in wt, but not in XBP-1^{-/-} MEF cells. Interestingly, however, Northern blot analysis revealed that MGP mRNA was dramatically down-regulated upon ER stress, indicating that the microarray data was incorrect. (FIG. 16A). In contrast, ERdj4 and p58^{IPK} expression was almost completely abolished in XBP-1^{-/-} cells (FIG. 16A). There are three isoforms of p58^{IPK} with transcripts of ˜6.5, 3.3 and 1.7 kb (Korth et al. 1996. Gene 170:181), all of which were XBP-1-dependent as assessed by using probes B and A specific for the 5′ (FIG. 16A) and 3′ regions of the gene, respectively (FIG. 16B). EST clone AI60401 3 represented the 3′ end of the 6.5 kb species of p58^{IPK} mRNA as confirmed by EST “walking” analysis. While probe A recognized only the 6.5 kb species, probe B hybridized to all three p58^{IPK}mRNAs, indicating that the ˜6.5 kb species shares 5′ ends with the other mRNA species.

The placement of the ERdj4 and p58^{IPK} genes downstream of XBP-1 was further established by examining their expression in MEFs lacking IRE1α (FIG. 16C). Consistent with the profound effect of XBP-1 on regulating ERdj4 expression was our finding that a construct containing 0.5 kb of ERdj4 promoter sequence fused to a luciferase reporter was induced by Tm as well as by cotransfected XBP-1s (FIG. 16D). Further, the ERdj4 promoter was not induced by Tm in XBP-1^{-/-} cells, while it was transactivated by cotransfected XBP-1s. Dependence of ERdj4 and p58^{IPK} expression on XBP-1 was also tested in primary B cells in which both XBP-1 transcription and posttranscriptional splicing to the XBP-1s form are induced during terminal B cell differentiation to plasma cells. This is functionally critical since lymphoid chimeras lacking XBP-1 fail to generate the plasma cell compartment. Both ERdj4 and p58^{ipk} were induced in LPS stimulated wild type cultures, but not in XBP-1 deficient B cells. In contrast, BiP was induced in both wild type and XBP-1 deficient B cells, although again, there was a modest impairment in its expression in the absence of XBP-1. XBP-1 transcripts were also examined in the mutant MEFs. This was possible because the disrupted XBP-1 gene produces a transcript, 0.4 kb longer than wt, composed of the neomycin gene and XBP-1 sequences arising from an alterative splicing event between a cryptic splice donor site in the neo cassette, inserted between exons 1 and 2, and the splice acceptor site for exon 3) (FIGS. 15A and 15C). XBP-1 mRNAs were induced by Tm treatment in both wild type and XBP-1 deficient MEF cells (FIGS. 15B, 16A), similar to what was reported for IRE-1 a deficient cells (Urano, F., et al. 2000a. J. Cell Sci. 113: 3697-3702; Urano, F., et al. 2000b. Science 287: 664-666). ATF6α was also modestly induced by Tm in these murine MEFs in contrast to what was observed in human HeLa cells (Yoshida, H., et al. 1998. J. Biol. Chem. 273: 33741-33749). However, the fold induction of both XBP-1 and ATF6α was modestly decreased in the absence of XBP-1 (FIGS. 15B and 16A). Although it is possible that the mutant transcript is aberrantly regulated, these results indicate some degree of autoregulation of XBP-1 as well as its crossregulation of ATF6α (FIGS. 15B, 16A).

These experiments identified both known and potentially novel UPR target genes and show that XBP-1 is essential for the expression of only some of them,. which include the DnaJ-like accessory proteins, ERdj4 and p58^{ipk}. It has a modest effect in regulating the expression of other known UPR target genes, BiP, ATF6α and itself, and no effect (CHOP, MGP) on other UPR genes.

### Example 18

### Identification of Genes Induced by XBP-1s

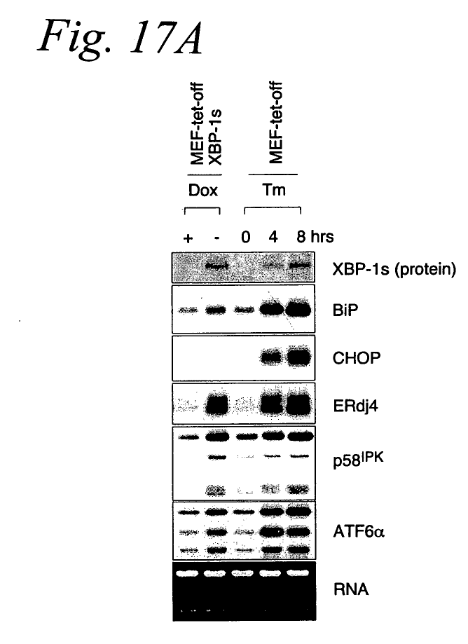

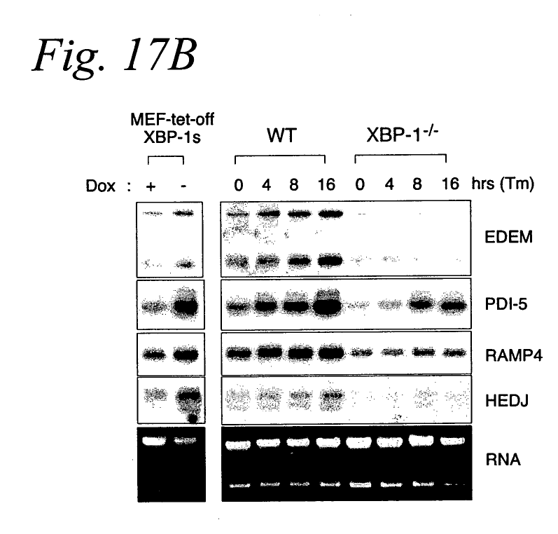

The minimally altered expression of some UPR target genes in XBP-1 deficient MEF cells indicates either that XBP-1 is not significantly involved in their expression or that there may be other transcription factor(s) that fully compensate for XBP-1. To examine whether XBP-1 itself is sufficient to induce these UPR target genes, the tet-off system was used to establish a cell line in which the spliced form of XBP-1, XBP-1s, is placed downstream of a tetracycline dependent promoter. XBP-1s was induced by removing doxycycline in the culture medium for three days. The expression level of the exogenous XBP-1s was comparable to endogenous XBP-1s in the parental MEF cells treated with Tm (FIG. 17A). To identify genes that were induced by XBP-1s, total RNAs were prepared from MEF-tet-off-XBP-1s cells cultured in the presence or absence of doxycycline and gene array analysis performed (Table 1). Consistent with the results from XBP-1^{-/-} cells, XBP-1s alone was sufficient to induce ERdj4 and p58^{ipk} expression. In addition, ˜23% of the total pool of analyzed UPR target genes were induced by XBP-1s, although the fold induction tended to be lower than upon Tm treatment. These UPR genes included Herp, BiP, Aremt, AW124049 EST and interferon beta. On the contrary, several UPR target genes including CHOP were not significantly induced by XBP-1s, suggesting that XBP-1s is either not involved in or not sufficient for their induction. The expression of BiP, CHOP, ERdj4 and p58^{ipk} was confirmed by Northern blot analysis (FIG. 17A). Induction of ERdj4 and p58^{ipk} by XBP-1s overexpression was comparable to that achieved by Tm treatment, while BiP was only marginally induced by XBP-1s. ATF6α was also induced by XBP-1s, as shown by Northern blot analysis, placing ATF6 downstream of XBP-1.

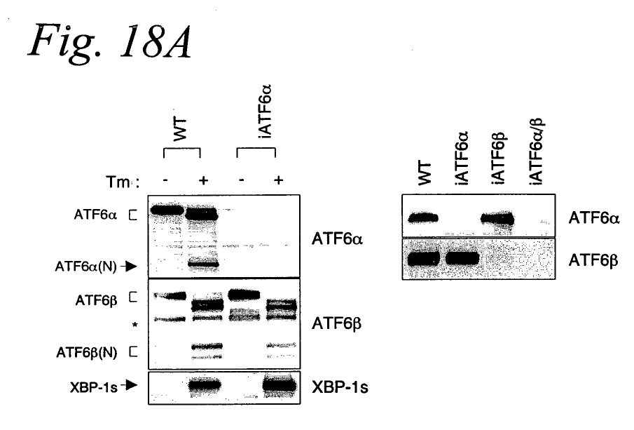

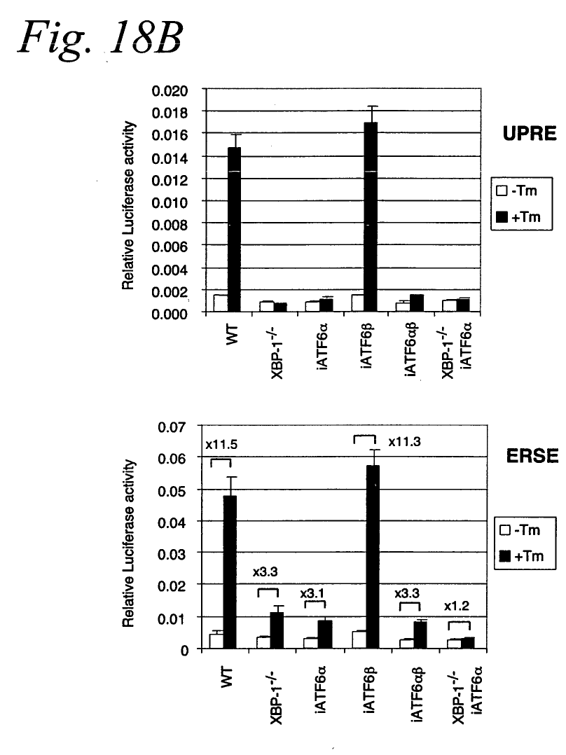

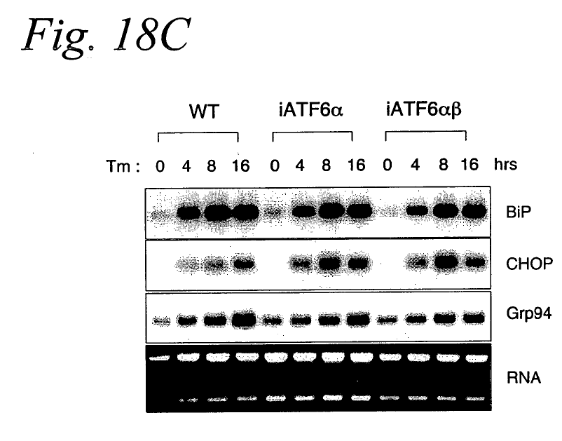

Further, several additional XBP-1 target genes, EDEM, protein disulfide isomerase-related protein P5 (PDI-P5), RAMP4, HEDJ, were identified by sorting using the criteria of inducibility by XBP-1s (Table 2). Strikingly, their expression was induced by Tm in wt, but not in XBP-1^{-/-} MEFs, confirming their XBP-1 dependency. XBP-1 dependent expression of EDEM is consistent with the recent finding that its induction was absent in MEFs lacking IRE1α (Yoshida, H., et al. Dev Cell 4: 265-271) (FIG. 17B). Two other genes, mgat-2 and BR140-like protein, identified in the microarray analysis, were not confirmed by Northern blot. Collectively, these results suggest that XBP-1 is essential for the regulation of several UPR target genes, ERdj4, p58^{IPK}, EDEM, PDI-P5, RAMP4, and HEDJ is modestly involved in the regulation of some UPR genes (BiP, XBP-1, ATF6α) and is not at all required for the expression of another subset of UPR target genes. Consistent with an important function for XBP-1 in regulating UPR target genes is the failure of Tm to induce the activity of either the UPRE or ERSE luciferase reporters in the XBP-1^{-/-} MEFS (see below, FIG. 18B).

### Example 19

### UPR Gene Expression in ATF6α and ATF6β Single and Double Deficient Cells

The ER transmembrane transcription factor ATF6α is proteolytically processed to release its active N-terminal region for nuclear transport upon ER stress, and has been reported to autonomously induce a subset of UPR target genes including BiP and CHOP (Yoshida, H., et al. J. Biol. Chem. 273: 33741-33749; Okada, T., et al. Biochem J 366: 585-594). Mice carrying a targeted deletion of the ATF6α gene are not available. To more directly assess the requirement of ATF6α in the UPR, its expression was therefore “knocked-down” in MEF cells by using an RNA polymerase III-driven siRNA expression plasmid. Cotransfection of the ATF6α specific siRNA vector with a multimerized ATF6 target site-1luciferase reporter (5×ATF6GL3) resulted in suppression of both ATF6-driven and Tm evoked luciferase expression, suggesting that ATF6α mRNA had been appropriately targeted by the siRNA vector. MEF cells were therefore stably transfected with the ATF6α siRNA vector to generate cell lines in which ATF6α expression was reduced (iATF6α). Suppression of ATF6α expression was confirmed by both Northern (not shown) and Western blot analysis (FIG. 18A). Wild type MEFs expressed three species of ATF6α mRNA of 8, 4.5 and 2.5 kb as reported previously (Zhu, C., et al. Mol Cell Biol 17: 4957-4966), and ATF6α mRNA levels were markedly decreased in knock down cells. In the parental wild type MEF line, ATF6α protein was synthesized as an ER resident 90 kDa precursor form and cleaved by S2P proteases to generate the 50 kDa active form upon ER stress, as expected (FIG. 18A). In contrast, ATF6α protein was not detected in the knocked-down cells, in either the absence or presence of Tm treatment (FIGS. 18A, B). Levels of XBP-1s and ATF6β transcripts (not shown) and protein (FIGS. 18A, B) were not altered in the iATF6α MEFs. This latter point is of interest as it suggests that XBP-1 is not downstream of ATF6α as previously suggested (Yoshida, H., et al. Mol Cell Biol 20: 6755-6767; Yoshida, H., et al. Cell 107: 881-891). Transient transfection assays revealed that neither the UPRE (5×ATF6GL3) reporter nor the ERSE reporter was induced at all in the iATF6α MEFs by Tm treatment (FIG. 15B). This cell line behaved in a manner consistent with a functional absence of ATF6α.

Gene array analysis was then performed on RNAs from these cell lines to identify UPR target genes whose expression was dependent on ATF6α. Surprisingly, the induction of almost all UPR target genes by Tm treatment was largely unaffected by ATF6α depletion (Table 3). In Northern blot analysis, BiP, CHOP, ERdj4 and p58^{ipk} transcripts were only modestly decreased in iATF6α MEFs, consistent with gene array results (FIG. 18C). These results show either that ATF6α is minimally involved in the regulation of UPR genes, or that there is functional redundancy. If the latter explanation is correct, then one possibility is that either ATF6β or XBP-1 can compensate for its loss.

The recently described ATF6β gene, which heterodimerizes with ATF6α (Haze, K., et al. Biochem J 355: 19-28) is closely related structurally to ATF6α, and is also a transmembrane ER protein. Upon activation by stress it is processed to an active, soluble form that translocates to the nucleus and transactivates endogenous BiP expression and the 5XAF6GL3 reporter. The strategy above was used to “knockdown” the expression of ATF6β in wt MEFs as well as in ATF6α MEFs to produce singly and doubly deficient cell lines. Northern and western (FIG. 18A, right panel) blot analysis revealed very reduced levels of ATF6β mRNA and protein in both cell lines. Surprisingly, the induction of UPR target genes BiP, CHOP, and Grp94, as assessed by Northern blot analysis, was normal, not only in the single ATF6β but also in the double ATF6α/β knockdown cell lines (FIG. 18C). This is consistent with the normal induction of the UPRE and ERSE reporters upon Tm treatment of iATF6β MEFs (FIG. 18B). Thus, in this system, neither ATFα nor β is required for the induction of UPR target genes. The impaired activity of the UPRE and ERSE reporter in response to Tm in iATFα and double iATFα/β reporters (FIG. 18B), however, suggests that there are additional UPR target genes regulated by ATF6.

### Example 20

### UPR Target Gene Expression in MEFs Lacking Both XBP-1 and ATF6α Largely Resembles that in Single XBP-1 Deficient MEFs.

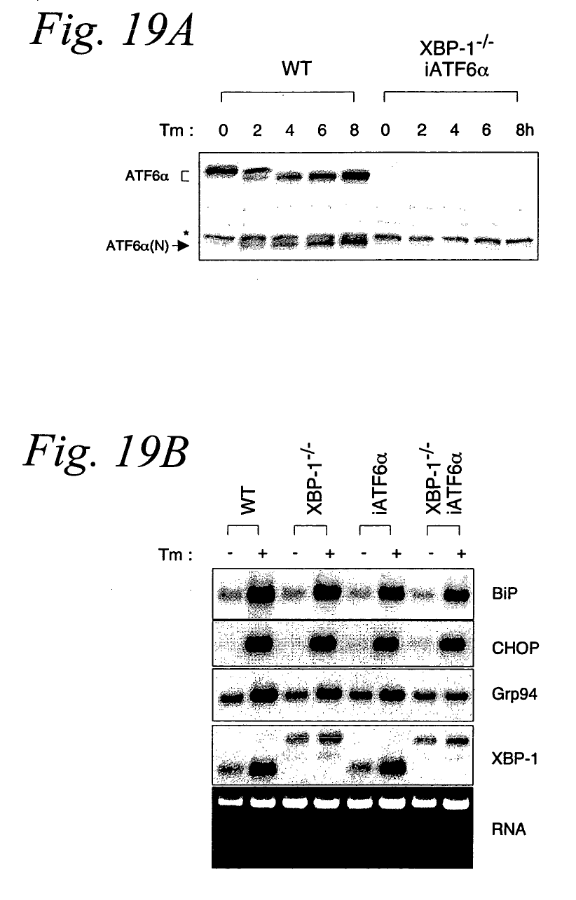

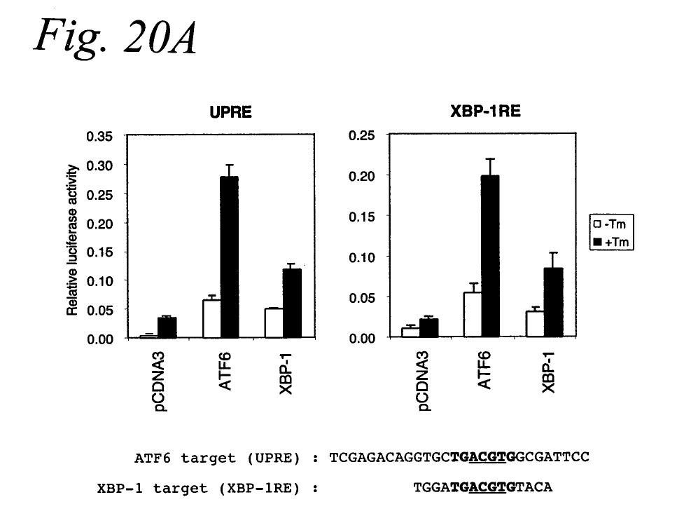

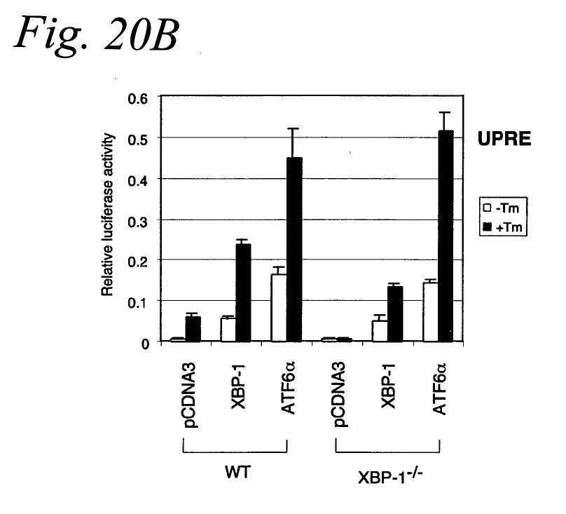

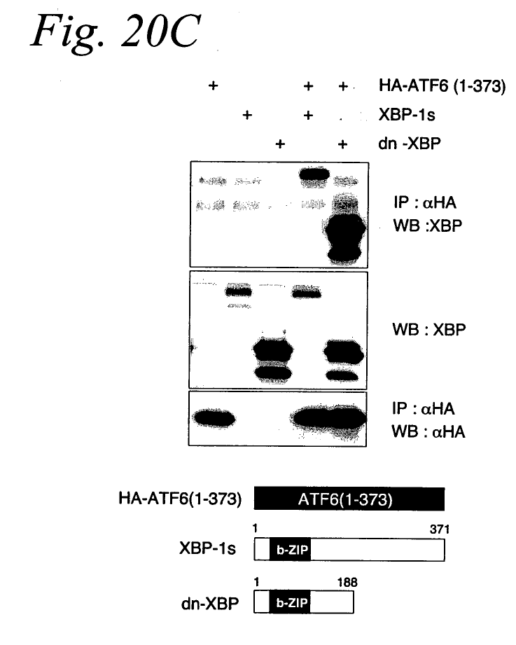

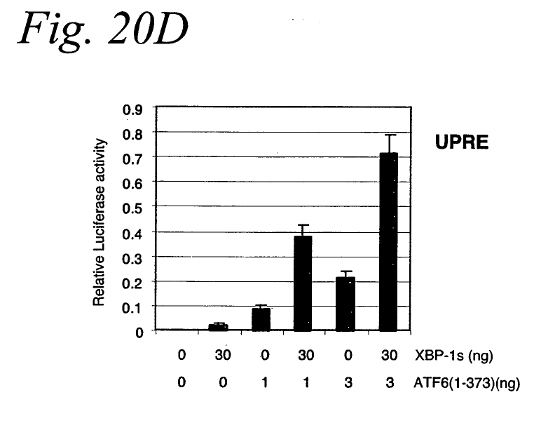

Whether there was functional redundancy between XBP-1 and ATF6α was also tested. XBP-1^{-/-} MEFs were transduced with the ATF6α RNA polymerase III-driven siRNA expression plasmid to generate MEFs that were doubly deficient in both XBP-1 and ATF6α, as shown by western blot analysis (FIG. 19A). Gene array and Northern blot analysis on RNA harvested from this MEF cell line in the presence or absence of Tm revealed that induction of most UPR target genes was still only minimally decreased in the absence of both XBP-1 and ATF6α (FIG. 19B). However, the induction of Bip and Grp94 by Tm, only modestly diminished in the XBP-1 or ATF6α singly deficient MEFs, was significantly suppressed in the double deficient MEFs (FIG. 20B). These experiments make two important points. First, neither of the currently known signaling pathways can account for the induction of several of the prototypical stress response genes, most notably CHOP and GADD45. Second, Bip and Grp94 are examples of a chaperone gene whose expression requires either, but not both, ATF6α or XBP-1. These doubly deficient cell lines are valuable reagents in searching for additional novel factors that can control target gene induction during the UPR.

### Example 21

### Interaction of XBP-1 and ATF6α

XBP-1 and ATF6α have both been implicated in the function of the UPR. While it has been shown by others that ATF6 is involved in the induction of a subset of UPR target genes (Zhu, C., Johansen, F. E., and Prywes, R. 1997. Mol Cell Biol 17: 4957-4966; Ye, J., et al. Mol Cell 6: 1355-1364; Yoshida, H., et al. 2000. Mol Cell Biol 20: 6755-6767; Yoshida, H., et al. 2001a. Cell 107: 881-891), the data in the instant examples do not substantiate that.

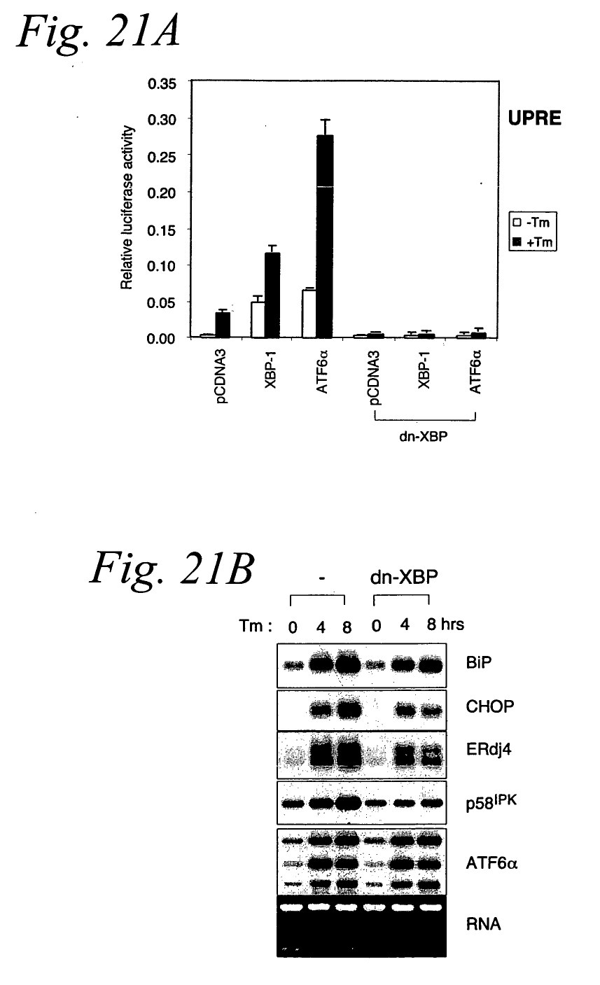

Other members of the basic region leucine zipper family of transcription factors form homodimers and heterodimers as typified by the c-Jun/c-Fos, c-Jun/ATF2 pairs (Sassone-Corsi, P.,et al. 1988. Nature 336: 692-695; Ivashkiv, L. B., et al. Mol. Cell. Biol. 10: 1609-1621). The ability of XBP-1 and ATF6α proteins to interact was tested. To test this possibility, coimmunoprecipitation experiments with overexpressed HA-tagged ATF6α (1-373) and XBP-1s in 293-T cells were performed. Immunoprecipitation with anti-HA antibody resulted in co-immunoprecipitation of XBP-1s as detected by immunoblotting with anti-XBP-1 antibody (FIG. 20C). A dominant negative version of XBP-1 (FIG. 21) which coimmunoprecipitates with both XBP-1 and ATF6α (FIG. 20C) was also generated. The N-terminal half of XBP-1s was sufficient for its interaction with ATF6α (1-373), indicating that the interaction between them occurred as expected through the leucine zipper domain. Similar to ATF6α/ATF6β heterodimerization, no association of endogenous XBP-1 and ATF6α was demonstrated, likely because of very low levels of these proteins. However, functional evidence for heterodimer formation was obtained by overexpression of XBP-1s and ATF6α (1-373). This resulted in modest synergistic transactivation of the UPRE reporter in transient co-transfection experiments, consistent with a more potent transactivation ability of heterodimeric as compared to homodimeric complexes (FIG. 20D). Results of transient transactivation assays, of course, must be interpreted in the context of studies that examine endogenous gene expression. Thus, XBP-1 and ATF6α likely form functionally relevant heterodimers.

### Example 22

### Dominant Negative XBP-1 Suppresses UPR Gene Induction

Since the N-terminal half (aa 1 to 188) of the XBP-1s protein lacks a transactivation domain but retains the leucine zipper motif essential for DNA binding and dimerization (FIG. 20C), it can function as a dominant negative that would inhibit not only XBP-1 and ATF6α but other putative factors that associated with them.

The function of this mutant was tested in reporter assays and dn-XBP completely abolished transactivation of the UPRE reporter by XBP-1s (FIG. 21A). Similarly, dn-XBP inhibited the transactivation of the UPRE reporter by ATF6α, demonstrating that dn-XBP dimerizes with both XBP-1 and with ATF6. A stable cell line overexpressing dn-XBP was generated to inhibit the function of both XBP-1 and ATF6α and examined its effect on endogenous UPR target gene expression. dn-XBP significantly suppressed the induction of XBP-1 target genes, ERdj4 and p58^{ipk} (FIG. 21B) although it did not completely inhibit XBP-1 and ATF6α activity as evidenced by the residual ERdj4 and p58^{ipk} expression in dn-XBP as opposed to XBP-1^{-/-} MEFs. Interestingly, it also suppressed the induction of CHOP (FIG. 21B). Considering that CHOP induction was not significantly influenced by either XBP-1 or ATF6α or β loss singly or doubly, we conclude that CHOP induction requires another leucine zipper transcription factor that associates with dn-XBP.

The UPR ensures the efficient translocation of newly synthesized peptides across the endoplasmic reticulum membrane and their subsequent folding, maturation and transport by activating the expression of chaperone genes. Two of the signaling systems that control the UPR are the IRE1/XBP-1 and ATF6 pathways. The relationship between XBP-1 and ATF6, two members of the basic region/leucine zipper class of transcription factors, has been unclear. This is in part because, in contrast to yeast, very few mammalian chaperone genes have been identified. DNA microarray analysis was used to search for genes regulated by XBP-1, and by ATF6α/β. Gene expression in MEFs derived from XBP-1 deficient embryos was compared to that in wildtype MEFs in the presence or absence of Tm, an agent that evokes the UPR. This analysis yielded several XBP-1-dependent genes, two of which were p58^{ipk} and ERdj4, members of the DnaJ/HSP40-like accessory gene family. However, the expression of members of the DnaK/Hsp70 family of genes such as BiP/Grp78 and CHOP, was only modestly dependent on XBP-1.

While several XBP-1 target genes could be identified, none of the UPR target genes analyzed were significantly affected by loss of ATF6α, ATF6β or both. Although no ATF6α dependent UPR target genes were found, the activity of the UPRE and ERSE reporters was completely absent or significantly suppressed in ATF6α knock-down cells. Furthermore, it has been shown that ATF6α (1-373) was sufficient for the induction of several UPR target genes, including BiP and CHOP (Okada et al. 2002 Biochem J 366: 585-594). Thus, the activity of ATF6α can be fully compensated by other UPR transcription factors, such as XBP-1, that share similar DNA binding specificity. XBP-1 dependent induction of p58^{IPK}, ERdj4 and HEDJ suggests that an important role of XBP-1 in the UPR is to control the expression of some cochaperones that activate ER resident HspTO proteins. Mice that lack XBP-1 die in utero from liver hypoplasia (Reimold et al. 2000 Genes Dev. 14: 152-157), while mice lacking XBP-1 in the lymphoid system fail to generate plasma cells and hence antibodies (Reimold et al. 1996 J. Exp. Med. 183: 393-401; Reimold et al. 2001 Nature 412: 300-307). The absolute dependence of genes, including ERdj4 and p58^{IPK}, EDEM, Ramp4, PDI-P5, and HEDJ, on XBP-1 for expression indicates that they will prove to have an important function in the UPR in plasma cells.

Moreover, ATF6α is situated downstream of XBP-1 since the induction of mouse ATF6α mRNA upon ER stress was partially compromised in the absence of XBP-1. However, given that the induction of ATF6α by XBP-1 is modest and that ATF6α is primarily regulated by post-translational mechanisms, these two factors are likely situated largely in parallel pathways.

### Equivalents

Those skilled in the art will recognize, or be able to ascertain using no more than routine experimentation, many equivalents to the specific embodiments and methods described herein. Such equivalents are intended to be encompassed by the scope of the following claims.

We claim:

1. A method of identifying compounds useful in modulating a biological activity of mammalian XBP-1 comprising,
a) providing an indicator composition comprising mammalian XBP-1 protein;
b) contacting the indicator composition with each member of a library of test compounds;
c) selecting from the library of test compounds a compound of interest that modulates the expression, processing, post-translational modification, and/or activity of XBP-1 protein; to thereby identify a compound that modulates a biological activity of mammalian XBP-1.

2. The method of claim 1, further comprising measuring the effect of the compound on the biological activity of XBP-1

3. The method of claim 1, wherein the biological activity is selected from the group consisting of: modulation of the UPR, modulation of cellular differentiation, modulation of IL-6 production, modulation of immunoglobulin production, modulation of the proteasome pathway, modulation of protein folding and transport, modulation of terminal B cell differentiation, and modulation of apoptosis.

4. The method of claim 1, wherein the post-translational modifications are selected from the group consisting of phophorylation, glycosylation and ubiquitination is modulated.

5. The method of claim 1, wherein the activity of XBP-1 is measured by measuring the binding of XBP-1 to IRE-1 or ATF6α.

6. The method of claim 1, wherein the activity of XBP-1 is measured by measuring the binding of XBP-1 to a regulatory region of a gene responsive to XBP-1.

7. The method of claim 6, wherein the gene is a chaperone gene.

8. The method of claim 6, wherein the gene is selected from the group consisting of ERdj4, p58^{ipk}, EDEM, PDI-P5, RAMP4, HEDJ, BiP, ATF6α, XBP-1, Armet and DNAJB9.

9. The method of claim 1, wherein the activity of XBP-1 is measured by measuring the production of a protein.

10. The method of claim 9, wherein the protein is selected from the group consisting of α-fetoprotein, α1-antitrypsin, and albumin.

11. The method of claim 9, wherein the protein is an immunoglobulin.

12. The method of claim 1, wherein the activity of XBP-1 is measured by measuring IL-6 expression.

13. The method of claim 1, wherein the indicator composition is a cell that expresses XBP-1 protein.

14. The method of claim 13, wherein the cell has been engineered to express the XBP-1 protein by introducing into the cell an expression vector encoding the XBP-1 protein.

15. The method of claim 1, wherein the indicator composition is a cell free composition.

16. The method of claim 1, wherein the indicator composition is a cell that expresses an XBP-1 protein and a target molecule, and the ability of the test compound to modulate the interaction of the XBP-1 protein with a target molecule is monitored.

17. The method of claim 1, wherein the indicator composition comprises an indicator cell, wherein the indicator cell comprises an XBP-1 protein and a reporter gene responsive to the XBP-1 protein.

18. The method of claim 17, wherein said indicator cell contains: a recombinant expression vector encoding the XBP-1 protein; and a vector comprising an XBP-1-responsive regulatory element operatively linked a reporter gene; and said method comprises:
a) contacting the indicator cell with a test compound;
b) determining the level of expression of the reporter gene in the indicator cell in the presence of the test compound; and
c) comparing the level of expression of the reporter gene in the indicator cell in the presence of the test compound with the level of expression of the reporter gene in the indicator cell in the absence of the test compound to thereby select a compound of interest that modulates the activity of XBP-1 protein.

19. A method of identifying compounds useful in modulating a biological activity of mammalian XBP-1 comprising,
a) providing an indicator composition comprising mammalian IRE-1 protein;
b) contacting the indicator composition with each member of a library of test compounds;
c) selecting from the library of test compounds a compound of interest that modulates the expression, processing, post-translational modification, and/or activity of the IRE-1 protein; to thereby identify a compound that modulates a biological activity of mammalian XBP-1.

20. The method of claim 19, wherein the biological activity is selected from the group consisting of: modulation of the UPR, modulation of cellular differentiation, modulation of IL-6 production, modulation of immunoglobulin production, modulation of the proteasome pathway, modulation of protein folding and transport, modulation of terminal B cell differentiation, and modulation of apoptosis.

21. The method of claim 19, wherein the activity of IRE-1 is a kinase activity.

22. The method of claim 19, wherein the activity of IRE-1 is an endoribonuclease activity.

23. The method of claim 19, wherein the activity of IRE-1 is measured by measuring the binding of IRE-1 to XBP-1.

24. The method of claim 19, wherein the indicator composition is a cell that expresses IRE-1 protein.

25. The method of claim 24, wherein the cell has been engineered to express the IRE-1 protein by introducing into the cell an expression vector encoding the IRE-1 protein.

26. The method of claim 19, wherein the indicator composition is a cell free composition.

27. The method of claim 19, wherein the indicator composition is a cell that expresses a mammalian IRE-1 protein and a target molecule, and the ability of the test compound to modulate the interaction of the IRE-1 protein with a target molecule is monitored.

28. A method of identifying a compound that modulates a mammalian XBP-1 biological activity comprising:
a) contacting cells deficient in XBP-1 or a molecule in a signaling pathway involving XBP-1 with a test compound; and
b) determining the effect of the test compound on the XBP-1 biological activity, the test compound being identified as a modulator of the biological activity based on the ability of the test compound to modulate the biological activity in the cells deficient in XBP-1 or a molecule in a signaling pathway involving XBP-1 to thereby identify a compound that modulates a mammalian XBP-1 biological activity.

29. The method of claim 28, wherein the cells are in a non-human animal deficient in XBP-1 or a molecule in a signal transduction pathway involving XBP-1 and the cells are contacted with the test compound by administering the test compound to the animal.

30. A method of identifying compounds useful in modulating a biological activity of mammalian XBP-1 comprising:
a) providing an indicator composition comprising mammalian XBP-1 or a molecule in a signal transduction pathway involving XBP-1;
b) contacting the indicator composition with each member of a library of test compounds;
c) selecting from the library of test compounds a compound of interest that modulates the expression, processing, post-translational modification, and/or activity of XBP-1 or the molecule in a signal transduction pathway involving XBP-1; to thereby identify a compound that modulates a biological activity of mammalian XBP-1.

31. The method of claim 30, wherein the indicator composition is a cell that expresses XBP-1, IRE-1, PERK, and/or ATF6α protein.

32. The method of claim 31, wherein the cell has been engineered to express the XBP-1, IRE-1, PERK, or ATF6α protein by introducing into the cell an expression vector encoding the XBP-1, IRE-1, PERK or ATF6α protein.

33. The method of claim 30, wherein the indicator composition is a cell free composition.

34. The method of claim 30, wherein the indicator composition is a cell that expresses an XBP-1, IRE-1, PERK, or ATF6α protein and a target molecule, and the ability of the test compound to modulate the interaction of the XBP-1, IRE-1, PERK or ATF6α protein with a target molecule is monitored.

35. A method of identifying a compound useful in modulating an autoimmune disease comprising:
a) providing an indicator composition comprising mammalian XBP-1 or a molecule in a signal transduction pathway involving XBP-1;
b) contacting the indicator composition with each member of a library of test compounds;
c) selecting from the library of test compounds a compound of interest that downmodulates the expression, processing, post-translational modification, and/or activity of XBP-1 or a molecule in a signal transduction pathway involving XBP-1; to thereby identify a compound that modulates an autoimmune disease.

36. The method of claim 35, wherein the activity of XBP-1 is measured by measuring the binding of XBP-1 to IRE-1 or ATF6α.

37. The method of claim 35, wherein the activity of XBP-1 is measured by measuring the binding of XBP-1 to a regulatory region of a gene responsive to XBP-1.

38. The method of claim 37, wherein the gene is a chaperone gene.

39. The method of claim 37, wherein the gene is selected from the group consisting of ERdj4, p58^{ipk}, EDEM, PDI-P5, RAMP4, HEDJ, BiP, ATF6α, XBP-1 and DNAJB9.

40. The method of claim 35, wherein the activity of XBP-1 is measured by measuring the production of a protein.

41. The method of claim 40, wherein the protein is selected from the group consisting of α-fetoprotein, albumin, α1-antitrypsin or an immunoglobulin.

42. The method of claim 35, wherein the activity of XBP-1 is measured by measuring IL-6 expression.

43. The method of claim 35, wherein the activity of IRE-1 is measured.

44. The method of claim 43, wherein the activity of IRE-1 is a kinase activity.

45. The method of claim 43, wherein the activity of IRE-1 is an endoribonuclease activity.

46. The method of claim 43, wherein the activity of IRE-1 is measured by measuring the binding of IRE-1 to XBP-1.

47. The method of claim 35, wherein the autoimmune disease is selected from the group consisting of: systemic lupus erythematosus; rheumatoid arthritis; goodpasture's syndrome; Grave's disease; Hashimoto's thyroiditis; pemphigus vulgaris; myasthenia gravis; scleroderma; autoimmune hemolytic anemia; autoimmune thrombocytopenic purpura; polymyositis and dermatomyositis; pernicious anemia; Sjögren's syndrome; ankylosing spondylitis; vasculitis, multiple sclerosis, inflammatory bowel disease, ulcerative colitis, Crohn's disease, and type I diabetes mellitus.

48. The method of claim 35, wherein the autoimmune disease involves the production of an antibody.

49. A method of identifying a compound useful in treating a malignancy comprising:
a) providing an indicator composition comprising mammalian XBP-1 or a molecule in a signal transduction pathway involving XBP-1;
b) contacting the indicator composition with each member of a library of test compounds;
c) selecting from the library of test compounds a compound of interest that modulates the expression, processing, post-translational modification, and/or activity of XBP-1 or a molecule in a signal transduction pathway involving XBP-1; to thereby identify a compound that modulates a malignancy.

50. The method of claim 49, wherein the activity of XBP-1 is measured by measuring the binding of XBP-1 to IRE-1.

51. The method of claim 49, wherein the activity of XBP-1 is measured by measuring the binding of XBP-1 to a regulatory region of a gene responsive to XBP-1.

52. The method of claim 51, wherein the gene is a chaperone gene.

53. The method of claim 51, wherein the gene is selected from the group consisting of ERdj4, p58^{ipk}, EDEM, PDI-P5, RAMP4, HEDJ, BiP, ATF6α, XBP-1, Armet and DNAJB9.

54. The method of claim 49, wherein the activity of XBP-1 is measured by measuring the production of a protein.

55. The method of claim 54, wherein the protein is selected from the group consisting of α-fetoprotein, albumin, α1-antitrypsin or an immunoglobulin.

56. The method of claim 54, wherein the activity of XBP-1 is measured by measuring IL-6 expression.

57. The method of claim 49, wherein the molecule in the signal transduction pathway is IRE-1 and the activity of IRE-1 is measured by measuring a kinase activity.

58. The method of claim 49, wherein the molecule in the signal transduction pathway is IRE-1 and the activity of IRE-1 is an endoribonuclease activity.

59. The method of claim 49, wherein the molecule in the signal transduction pathway is IRE-1 and the activity of IRE-1 is measured by measuring the binding of IRE-1 to XBP-1.

60. The method of claim 49, wherein the malignancy is selected from the group consisting of: acute lymphoblastic leukemia; acute myeloid leukemia; adrenocortical carcinoma; AIDS-related lymphoma; cancer of the bile duct; bladder cancer; bone cancer, osteosarcomal malignant fibrous histiocytomal brain stem gliomal brain tumor; breast cancer; bronchial adenomas; carcinoid tumors; adrenocortical carcinoma; central nervous system lymphoma; cancer of the sinus, cancer of the gall bladder; gastric cancer; cancer of the salivary glands; cancer of the esophagus; neural cell cancer; intestinal cancer (e.g., of the large or small intestine); cervical cancer; colon cancer; colorectal cancer; cutaneous T-cell lymphoma; B-cell lymphoma; T-cell lymphoma; endometrial cancer; epithelial cancer; endometrial cancer; intraocular melanoma; retinoblastoma; hairy cell leukemia; liver cancer; Hodgkin's disease; Kaposi's sarcoma; acute lymphoblastic leukemia; lung cancer; non-Hodgkin's lymphoma; melanoma; multiple myeloma; neuroblastoma; prostate cancer; retinoblastoma; Ewing's sarcoma; vaginal cancer; Waldenstrom's macroglobulinemia; adenocarcinomas; ovarian cancer, chronic lymphocytic leukemia, pancreatic cancer; and Wilm's tumor.

61. The method of claim 49, wherein the malignancy is in a secretory cell.

62. A method for identifying a compound which modulates an interaction between mammalian XBP-1 and mammalian IRE-1 comprising:
(a) providing a first polypeptide comprising a IRE-1 interacting portion of an XBP-1 molecule and a second polypeptide comprising an XBP-1 interacting portion of an IRE-1 molecule in the presence and the absence of a plurality of test compounds; and
(b) determining the degree of interaction between the first and the second polypeptide in the presence and the absence of a test compound to thereby identify a compound which modulates an interaction between mammalian XBP-1 and mammalian IRE-1.

63. A method for identifying a compound which modulates an interaction between mammalian XBP-1 and mammalian ATF6α comprising:
(a) providing a first polypeptide comprising a ATF6α interacting portion of an XBP-1 molecule and a second polypeptide comprising an XBP-1 interacting portion of an ATF6oc molecule in the presence and the absence of a plurality of test compounds; and
(b) determining the degree of interaction between the first and the second polypeptide in the presence and the absence of a test compound to thereby identify a compound which modulates an interaction between mammalian XBP-1 and mammalian ATF6α.

64. The method of claim 62 or 63, wherein the interaction between the first and second peptides is determined by binding of XBP-1 to IRE-1 or ATF6α.

65. The method of claim 62 or 63, wherein the interaction between the first and second peptides is determined by measuring XBP-1 activity.

66. The method of claim 62 or 63, wherein the interaction between the first and second peptides is determined by measuring the level of spliced XBP-1.

67. The method of claim 62 or 63, wherein the interaction between the first and second peptides is determined by measuring the level of unspliced XBP-1.

68. The method of claim 62 or 63, wherein the compound is useful to treat autoimmune diseases.

69. The method of claim 62 or 63, wherein the compound is useful to treat malignancies.

70. The method of claim 62 or 63, wherein the compound is useful to modulate a biological activity of XBP-1.

71. A recombinant cell comprising an exogenous mammalian XBP-1 molecule or a portion thereof comprising the nucleotide sequence of XBP-1 spanning the splice junction, and a reporter gene operably linked to a regulatory region responsive to spliced XBP-1 such that upon splicing of the XBP-1 protein, transcription of the reporter gene occurs.

72. A method of detecting the ability of a compound to upmodulate splicing of mammalian XBP-1 comprising, contacting the cell of claim 68 with a compound and measuring the expression of the reporter gene in the presence and the absence of the compound, wherein an increase in the level of spliced XBP-1 in the presence of the compound indicates that the compound upmodulates splicing of mammalian XBP-1.

73. A method for modulating expression and/or activity of mammalian XBP-1 in a cell comprising contacting the cell with an agent that modulates expression and/or activity of a protein that activates XBP-1 to thereby regulate the expression and/or activity of mammalian XBP-1.

74. The method of claim 73, wherein the protein that activates XBP-1 is IRE-1.

75. The method of claim 73, wherein the agent is not a proteasome inhibitor of the dipeptidyl boronate class.

76. The method of claim 73, wherein the cell is a cell from a patient identified as one in need of modulation of the UPR.

77. The method of claim 76, wherein the patient is identified by measuring expression, processing, post-translational modification, and/or activity of XBP-1 protein or a protein in a signal transduction pathway involving XBP-1.

78. A method for modulating expression, in a cell, of a gene whose transcription is regulated by mammalian XBP-1, comprising contacting the cell with an agent that increases expression, processing, post-translational modification, and/or activity of spliced XBP-1 such that expression of the gene is altered.

79. The method of claim 78, wherein the cell is a cell isolated from or present in a patient identified as one in need of modulation of the UPR.

80. The method of claim 78, wherein the patient is identified by measuring expression, processing, post-translational modification, and/or activity of XBP-1 protein or a protein in a signal transduction pathway involving XBP-1.

81. A method for modulating expression, in a cell, of a gene whose transcription is regulated by mammalian XBP-1, comprising contacting the cell with an agent that increases the ratio of spliced XBP-1 to unspliced XBP-1 such that expression of the gene is altered.

82. The method of claim 81, wherein the cell is a cell isolated from or present in a patient identified as one in need of modulation of the UPR.

83. The method of claim 81, wherein the patient is identified by measuring expression, processing, post-translational modification, and/or activity of XBP-1 protein or a protein in a signal transduction pathway involving XBP-1.

84. The method of any of claims 78-81, wherein the step of contacting occurs in vivo in a subject that would benefit from modulation of an XBP-1 biological activity.

85. A method for increasing expression, in a mammalian cell, of a gene involved in mediating a biological effect of XBP-1 whose transcription is regulated by XBP-1, comprising contacting a cell with an agent that modulates expression, processing, post-translational modification, and/or activity of XBP-1 in the cell such that expression of the gene is increased.

86. The method of claim 85, wherein the cell is isolated from or present in a patient identified as one in need of modulation of the UPR.

87. The method of claim 85, wherein the patient is identified by measuring expression, processing, post-translational modification, and/or activity of XBP-1 protein or a protein in a signal transduction pathway involving XBP-1.

88. A method for decreasing expression, in a mammalian cell, of a gene involved in mediating a biological effect of XBP-1 whose transcription is regulated by XBP-1, comprising contacting a cell with an agent that modulates expression, processing, post-translational modification, and/or activity of XBP-1 in the cell such that expression of the gene is decreased.

89. The method of claim 88, wherein the agent is not a proteasome inhibitor of the dipeptidyl boronate class.

90. The method of claim 88, wherein the cell is isolated from or present in a patient identified as one in need of modulation of the UPR.

91. The method of claim 90, wherein the patient is identified by measuring expression, processing, post-translational modification, and/or activity of XBP-1 protein or a protein in a signal transduction pathway involving XBP-1.

92. A method for increasing expression, in a mammalian cell, of a gene involved in mediating a biological effect of XBP-1 whose transcription is regulated by XBP-1, comprising contacting a cell with an agent that increases the activity of spliced XBP-1 in the cell such that expression of the gene is increased.

93. The method of claim 92, wherein the cell is isolated from or present in a patient identified as one in need of modulation of the UPR.

94. The method of claim 92, wherein the patient is identified by measuring expression, processing, post-translational modification, and/or activity of XBP-1 protein or a protein in a signal transduction pathway involving XBP-1.

95. A method for increasing expression, in a mammalian cell, of a gene involved in mediating a biological effect of XBP-1 whose transcription is regulated by XBP-1, comprising contacting a cell with an agent that decreases the activity of unspliced XBP-1 in the cell such that expression of the gene is increased.

96. The method of claim 95, wherein the activity of unspliced XBP-1 comprises inhibiting the activity of spliced XBP-1.

97. A method for decreasing expression, in a mammalian cell, of a gene involved in mediating a biological effect of XBP-1 whose transcription is regulated by XBP-1, comprising contacting a cell with an agent that decreases the activity of spliced XBP-1 in the cell such that expression of the gene is decreased.

98. The method of claim 97, wherein the agent is not a proteasome inhibitor of the dipeptidyl boronate class.

99. The method of claim 97, wherein the cell is isolated from or present in a patient identified as one in need of modulation of the UPR.

100. The method of claim 99, wherein the patient is identified by measuring expression, processing, post-translational modification, and/or activity of XBP-1 protein or a protein in a signal transduction pathway involving XBP-1.

101. The method of claim 97, wherein the activity of spliced XBP-1 is decreased by introducing a dominant negative XBP-1 protein or nucleic acid molecule that mediates RNAi into the cell in an amount sufficient to inhibit activity of spliced XBP-1.

102. A method for decreasing expression, in a mammalian cell, of a gene involved in mediating a biological effect of XBP-1 whose transcription is regulated by XBP-1, comprising contacting a cell with an agent that increases the activity of unspliced XBP-1 in the cell such that expression of the gene is decreased.

103. The method of claim 102, wherein the activity of unspliced XBP-1 comprises inhibiting the activity of spliced XBP-1.

104. The method of any of claims 86, 90, 93, 95, 99, 102, wherein the gene is selected from the group consisting of ERdj4, p58^{ipk}, EDEM, PDI-P5, RAMP4, HEDJ, BiP, ATF6α, XBP-1, Armet and DNAJB9.

105. The method of any of claims 86, 90, 93, 95, 99, 102, wherein the cell is a B cell.

106. A method of modulating at least one mammalian XBP-1 biological activity comprising contacting a cell with an agent that increases the expression, processing, post-translational modification, and/or activity of spliced XBP-1 in a cell such that at least one biological activity of mammalian XBP-1 is modulated.

107. The method of claim 106, wherein the cell is isolated from or present in a patient identified as one in need of modulation of the UPR.

108. The method of claim 107, wherein the patient is identified by measuring expression, processing, post-translational modification, and/or activity of XBP-1 protein or a protein in a signal transduction pathway involving XBP-1.

109. A method of modulating at least one mammalian XBP-1 biological activity comprising contacting a cell with an agent that decreases the expression, processing, post-translational modification, and/or activity of spliced XBP-1 in a cell such that the biological activity is modulated.

110. The method of claim 109, wherein the agent is not a proteasome inhibitor of the dipeptidyl boronate class.

111. The method of claim 109, wherein the cell is isolated from or present in a patient identified as one in need of modulation of the UPR.

112. The method of claim 109, wherein the patient is identified by measuring expression, processing, post-translational modification, and/or activity of XBP-1 protein or a protein in a signal transduction pathway involving XBP-1.

113. A method of modulating cellular differentiation comprising contacting a mammalian cell with an agent that increases the expression, processing, post-translational modification, and/or activity of unspliced XBP-1 such that the biological response is modulated.

114. The method of claim 113, wherein the cell is isolated from or present in a patient identified as one in need of modulation of the UPR.

115. The method of claim 114, wherein the patient is identified by measuring expression, processing, post-translational modification, and/or activity of XBP-1 protein or a protein in a signal transduction pathway involving XBP-1.

116. A method for downmodulating, in mammalian cells, the level of expression of genes which are activated by extracellular influences which induce a signal transduction pathway involving XBP-1, the method comprising contacting a cell with an agent that reduces the expression, processing, post-translational modification, and/or activity of spliced XBP-1 in the cells such that expression of said genes is reduced.

117. The method of claim 116, wherein the agent is not a proteasome inhibitor of the dipeptidyl boronate class.

118. The method of claim 116, wherein the cell is isolated from or present in a patient identified as one in need of modulation of the UPR.

119. The method of claim 118, wherein the patient is identified by measuring expression, processing, post-translational modification, and/or activity of XBP-1 protein or a protein in a signal transduction pathway involving XBP-1.

120. A method for upmodulating, in mammalian cells, the level of expression of genes which are activated by extracellular influences which induce a signal transduction pathway involving XBP-1, the method comprising contacting a cell with an agent that increases the expression, processing, post-translational modification, and/or activity of spliced XBP-1 in the cells such that expression of said genes is upmodulated.

121. The method of claim 120, wherein the cell is isolated from or present in a patient identified as one in need of modulation of the UPR.

122. The method of claim 120, wherein the patient is identified by measuring expression, processing, post-translational modification, and/or activity of XBP-1 protein or a protein in a signal transduction pathway involving XBP-1.

123. The method of claim 120, wherein the extracellular influence induces ER stress.

124. A method for downmodulating XBP-1-mediated intracellular signaling in a mammalian cell comprising contacting the cell with an agent that downmodulates the expression, processing, post-translational modification, and/or activity of spliced XBP-1 in the cell such that XBP-1 mediated intracellular signaling is downmodulated.

125. The method of claim 124, wherein the agent is not a proteasome inhibitor of the dipeptidyl boronate class.

126. The method of claim 124, wherein the cell is isolated from or present in a patient identified as one in need of modulation of the UPR.

127. The method of claim 126, wherein the patient is identified by measuring expression, processing, post-translational modification, and/or activity of XBP-1 protein or a protein in a signal transduction pathway involving XBP-1.

128. A method for upmodulating XBP-1-mediated intracellular signaling comprising contacting a mammalian cell with an agent that upmodulates the expression, processing, post-translational modification, and/or activity of spliced XBP-1 in the cell such that XBP-1 mediated intracellular signaling is upmodulated.

129. The method of claim 128, wherein the cell is isolated from or present in a patient identified as one in need of modulation of the UPR.

130. The method of claim 129, wherein the patient is identified by measuring expression, processing, post-translational modification, and/or activity of XBP-1 protein or a protein in a signal transduction pathway involving XBP-1.

131. A method of increasing IL-6 expression in a cell comprising contacting a cell with an agent that increases the activity of spliced XBP-1 in the cell such that IL-6 production is increased.

132. A method of increasing IL-6 production in a mammalian cell comprising contacting a cell with an agent that decreases the activity of unspliced XBP-1 in the cell such that IL-6 production is increased.

133. A method of decreasing IL-6 production in a mammalian cell comprising contacting a cell with an agent that decreases the activity of spliced XBP-1 in the cell such that IL-6 production is decreased.

134. A method of decreasing IL-6 production in a mammalian cell comprising contacting a cell with an agent that increases the activity of unspliced XBP-1 in the cell such that IL-6 production is decreased.

135. A method of downmodulating apoptosis in a mammalian cell comprising contacting a cell with an agent that increases the expression, processing, post-translational modification, and/or activity of spliced XBP-1 in the cell such that apoptosis is decreased.

136. A method of upmodulating apoptosis in a mammalian cell comprising contacting a cell with an agent that decreases the expression, processing, post-translational modification, and/or activity of spliced XBP-1 in the cell in the cell such that apoptosis is upmodulated.

137. The method of claim 136, wherein the agent is not a proteasome inhibitor of the dipeptidyl boronate class.

138. The method of claim 136, wherein the cell is isolated from or present in a patient identified as one in need of modulation of the UPR.

139. The method of claim 138, wherein the patient is identified by measuring expression, processing, post-translational modification, and/or activity of XBP-1 protein or a protein in a signal transduction pathway involving XBP-1.

140. A method of increasing protein folding, transport, and/or secretion comprising contacting a cell with an agent that increases the expression, processing, post-translational modification, and/or activity of spliced XBP-1 in the cell such that the production of the protein is increased.

141. The method of claim 140, wherein the protein is a viral protein.

142. The method of claim 140, wherein the increased protein folding or transport is measured by increased chaperone protein production.

143. The method of claim 140, wherein the protein is selected from the group consisting of α-fetoprotein, albumin, α1-antitrypsin and luciferase.

144. The method of claim 140, wherein the protein is exogenous to the cell.

145. The method of claim 140, wherein the protein is an immunoglobulin.

146. The method of claim 140, wherein the cell is a B cell

147. The method of claim 140, wherein the cell is a hepatocyte.

148. The method of claim 140, wherein the protein is recombinantly expressed in a cell.

149. A method of increasing protein folding or transport comprising contacting a cell with an agent that decreases the expression, processing, post-translational modification, and/or activity of unspliced XBP-1 in the cell such that production of the protein is increased.

150. A method of decreasing protein folding or transport in a cell comprising contacting a cell with an agent that increases the expression, processing, post-translational modification, and/or activity of unspliced XBP-1 in the cell such that production of the protein is decreased.

151. The method of claim 150, wherein the agent is not a proteasome inhibitor of the dipeptidyl boronate class.

152. The method of claim 150, wherein the cell is isolated from or present in a patient identified as one in need of modulation of the UPR.

153. The method of claim 150, wherein the patient is identified by measuring expression, processing, post-translational modification, and/or activity of XBP-1 protein or a protein in a signal transduction pathway involving XBP-1.

154. A method of modulating terminal B cell differentiation comprising contacting a mammalian cell with an agent that modulates IL-4 induced signaling in a B cell such that XBP-1 induced transcription is modulated, to thereby modulate terminal B cell differentiation.

155. The method of claim 154, wherein the cell is isolated from or present in a patient identified as one in need of modulation of the UPR.

156. The method of claim 155, wherein the patient is identified by measuring expression, processing, post-translational modification, and/or activity of XBP-1 protein or a protein in a signal transduction pathway involving XBP-1.

157. A method of modulating an XBP-1 biological activity in a mammalian cell comprising contacting a cell with an agent that induces terminal B cell differentiation.

158. The method of claim 157, wherein the cell is isolated from or present in a patient identified as one in need of modulation of the UPR.

159. The method of claim 157, wherein the patient is identified by measuring expression, processing, post-translational modification, and/or activity of XBP-1 protein or a protein in a signal transduction pathway involving XBP-1.

160. The method of claim 157, wherein the agent is IL-4.

161. The method of claim 157, wherein the agent acts via the signaling protein, STAT6.

162. The method of claim 157, wherein the agent is one or more agents selected from the group consisting of: LPS, CD40 and IL-4.

163. A method of treating or preventing a disorder that could benefit from treatment with an agent that dowmnodulates the activity of spliced XBP-1 or a molecule in a signal transduction pathway involving XBP-1 in a mammalian subject comprising administering to the subject with said disorder an agent that downmodulates the expression, processing, post-translational modification, and/or activity of XBP-1 or a molecule in a signal transduction pathway involving XBP-1.

164. The method of claim 75, wherein the patient is identified by measuring expression, processing, post-translational modification, and/or activity of XBP-1 protein or a protein in a signal transduction pathway involving XBP-1.

165. The method of claim 163, wherein the agent modulates the ratio of unspliced XBP-1 to spliced XBP-1.

166. The methods of claim 163, wherein the disorder is an autoimmune disease.

167. The method of claim 111, wherein the disorder is a malignancy.

168. A method of treating or preventing a malignancy comprising administering to a mammalian subject with said malignancy an agent that downmodulates the expression, processing, post-translational modification, and/or activity of XBP-1 or a molecule in a signal transduction pathway involving XBP-1 further comprising administering an additional agent useful in treating the malignancy.

169. The method of claim 168, wherein the additional agent is a proteasome inhibitor of the dipeptidyl boronate class.

170. A method of treating or preventing a disorder that could benefit from treatment with an agent that upmodulates the activity of spliced XBP-1 or a molecule in a signal transduction pathway involving XBP-1 in a subject comprising administering to the subject with said disorder an agent that upmodulates the expression, processing, post-translational modification, and/or activity of XBP-1 or a molecule in a signal transduction pathway involving XBP-1.

171. The method of claim 170, wherein the agent modulates the ratio of unspliced XBP-1 to spliced XBP-1.

172. The methods of claim 170, wherein the disorder is an acquired immunodeficiency disorder or an infectious disease.

173. An immunomodulatory composition comprising a nucleic acid molecule encoding spliced mammalian XBP-1 and an antigen.

174. An immunomodulatory composition comprising a compound that increases spliced mammalian XBP-1 activity and an antigen.

175. An immunomodulatory composition comprising an inhibitor of spliced mammalian XBP-1 and an antigen.

176. The immunomodulatory composition of claim 175, wherein the inhibitor is a dominant negative inhibitor of spliced mammalian XBP-1 and an antigen.

177. A method for modulating an autoimmune disease in a subject comprising administering the immunomodulatory compositions of any one of claims 173-175.

178. A method for modulating cellular differentiation in a mammalian subject comprising administering the immunomodulatory compositions of any one of 173-175.

179. A method for enhancing an immune response in a mammalian subject comprising administering a nucleic acid molecule encoding spliced XBP-1 to the subject such that the immune response is enhanced.

180. A method of enhancing an immune response in a mammalian subject comprising administering an XBP-1 agonist to the subject such that the immune response is enhanced.


SEQUENCE LISTING (19 sequences)

SEQ ID NO: 1 - DNA, 26 bp, Artificial Sequence
  Synthetic construct
cagcactcagactacgtgcacctctg

SEQ ID NO: 2 - DNA, 15 bp, Artificial Sequence
  Synthetic construct
tggatgacgtgtaca

SEQ ID NO: 3 - DNA, 28 bp, Artificial Sequence
  Synthetic construct
tcgagacaggtgctgacgtggcgattcc

SEQ ID NO: 4 - DNA, 19 bp, Artificial Sequence
  Synthetic construct
ccaatnnnnnnnnnccacg

SEQ ID NO: 5 - DNA, 20 bp, Artificial Sequence
  Synthetic construct
acacgcttgggaatggacac

SEQ ID NO: 6 - DNA, 20 bp, Artificial Sequence
  Synthetic construct
ccatgggaagatgttctggg

SEQ ID NO: 7 - DNA, 21 bp, Artificial Sequence
  Synthetic construct
gacgtttcctggctatggtgg

SEQ ID NO: 8 - DNA, 23 bp, Artificial Sequence
  Synthetic construct
caggcctatgctatcctctaggc

SEQ ID NO: 9 - DNA, 15 bp, Artificial Sequence
  Synthetic construct
tggatgacgtgtaca

SEQ ID NO: 10 - DNA, 28 bp, Artificial Sequence
  Synthetic construct
tcgagacaggtgctgacgtggcgattcc

SEQ ID NO: 11 - DNA, 21 bp, Artificial Sequence
  Synthetic construct
gggattcatgaatggccctta

SEQ ID NO: 12 - DNA, 64 bp, Artificial Sequence
  Synthetic construct
cgcgtggatgacgtgtacatggatgacgtgtacatggatgacgtgtacatggatgacgtgtaca

SEQ ID NO: 13 - DNA, 64 bp, Artificial Sequence
  Synthetic construct
gatctgtacacgtcatccatgtacacgtcatccatgtacacgtcatccatgtacacgtcatcca

SEQ ID NO: 14 - DNA, 51 bp, Artificial Sequence
  Synthetic construct
cgcgtcaccaatcggaggcctccacgaccaccaatcggaggcctccacgac

SEQ ID NO: 15 - DNA, 26 bp, Artificial Sequence
  Synthetic construct
aggcttgggctctaatggcctctcaa

SEQ ID NO: 16 - DNA, 20 bp, Artificial Sequence
  Synthetic construct
ctccgaacgccgagtagcct

SEQ ID NO: 17 - DNA, 53 bp, Artificial Sequence
  Synthetic construct
ggcagtgtcgcctggtgttgaagcttcaacaccaggcgacactgccctttttg

SEQ ID NO: 18 - DNA, 57 bp, Artificial Sequence
  Synthetic construct
aattcaaaaagggcagtgtcgcctggtgttgaagcttcaacaccaggcgacactgcc

SEQ ID NO: 19 - DNA, 22 bp, Artificial Sequence
  Synthetic construct
gggtggcagaagtcagtttatg